(19) United States
(12) Patent Application Publication
(10) Pub. No.: US 2007/0197530 A1
(43) Pub. Date: Aug. 23, 2007

(54) Amido compounds and their use as pharmaceuticals

(76) Inventors: Yun-Long Li, Wilmington, DE (US); Wenqing Yao, Kennett Square, PA (US); Jincong Zhuo, Boothwyn, PA (US)

Correspondence Address: FISH & RICHARDSON PC, P.O. BOX 1022, MINNEAPOLIS, MN, 55440-1022, US

(21) Appl. No.: 11/699,826

(22) Filed: Jan. 30, 2007

Related U.S. Application Data
(60) Provisional application No. 60/763,726, filed on Jan. 31, 2006.
(60) Provisional application No. 60/808,680, filed on May 26, 2006.

(51) Int. Cl. A61K 31/5377; A61K 31/496; A61K 31/4545; A61K 31/454; C07D 413/02; C07D 401/02; C07D 403/02

(52) U.S. Cl. 514/235.2; 514/316; 514/326; 514/253.11; 544/128; 544/360; 546/209; 546/208; 546/192

(57) ABSTRACT

The present invention relates to inhibitors of 11-β hydroxyl steroid dehydrogenase type 1 and pharmaceutical compositions thereof. The compounds of the invention can be useful in the treatment of various diseases associated with expression or activity of 11-β hydroxyl steroid dehydrogenase type 1.

### CROSS REFERENCE TO RELATED APPLICATIONS

This application claims the benefit of U.S. Ser. Nos. 60/763,726, filed January 31, 2006, and 60/808,680, filed May 26, 2006, the disclosures of each of which are incorporated herein by reference 1 0 in their entireties.

### FIELD OF THE INVENTION

The present invention relates to modulators of 11-β hydroxyl steroid dehydrogenase type 1 (11βHSD1), compositions thereof, and methods of using the same.

### BACKGROUND OF THE INVENTION

Glucocorticoids are steroid hormones that have the ability to modulate a plethora of biological processes including development, neurobiology, inflammation, blood pressure, and metabolism. In humans, the primary endogenously produced glucocorticoid is cortisol. Two members of the nuclear hormone receptor superfamily, glucocorticoid receptor (GR) and mineralcorticoid receptor (MR), are the key mediators of cortisol function in vivo. These receptors possess the ability to directly modulate transcription via DNA-binding zinc finger domains and transcriptional activation domains. This functionality, however, is dependent on the receptor having first bound to ligand (cortisol); as such, these receptors are often referred to as ‘ligand-dependent transcription factors’.

Cortisol is synthesized in the zona fasciculate of the adrenal cortex under the control of a short-term neuroendocrine feedback circuit called the hypothalamic-pituitary-adrenal (HPA) axis. Adrenal production of cortisol proceeds under the control of adrenocorticotrophic hormone (ACTH), a factor produced and secreted by the anterior pituitary. Production of ACTH in the anterior pituitary is itself highly regulated, being driven by corticotropin releasing hormone (CRH) produced by the paraventricular nucleus of the hypothalamus. The HPA axis functions to maintain circulating cortisol concentrations within restricted limits, with forward drive at the diurnal maximum or during periods of stress being rapidly attenuated by a negative feedback loop resulting from the ability of cortisol to suppress ACTH production in the anterior pituitary and CRH production in the hypothalamus.

The importance of the HPA axis in controlling glucocorticoid excursions is evident from the fact that disruption of this homeostasis by either excess or deficient secretion or action results in Cushing's syndrome or Addison's disease, respectively (Miller and Chrousos (2001) Endocrinology and Metabolism, eds. Felig and Frohman (McGraw-Hill, New York), 4^{th} Ed.: 387-524). Interestingly, the phenotype of Cushing's syndrome patients closely resembles that of Reaven's metabolic syndrome (also known as Syndrome X or insulin resistance syndrome) including visceral obesity, glucose intolerance, insulin resistance, hypertension, and hyperlipidemia (Reaven (1993) Ann. Rev. Med. 44: 121-131). Paradoxically, however, circulating glucocorticoid levels are typically normal in metabolic syndrome patients.

For decades, the major determinants of glucocorticoid action were believed to be limited to three primary factors: 1) circulating levels of glucocorticoid (driven primarily by the HPA axis), 2) protein binding of glucocorticoids in circulation (upward of 95%), and 3) intracellular receptor density inside target tissues. Recently, a fourth determinant of glucocorticoid function has been identified: tissue-specific pre-receptor metabolism. The enzymes 11-beta hydroxysteroid dehydrogenase type 1 (11βHSD1) and 11-beta hydroxysteroid dehydrogenase type 2 (11βHSD2) catalyze the interconversion of active cortisol (corticosterone in rodents) and inactive cortisone (11-dehydrocorticosterone in rodents). 11βHSD1 has been shown to be an NADPH-dependent reductase, catalyzing the activation of cortisol from inert cortisone (Low et al. (1994) J. Mol. Endocrin. 13: 167-174); conversely, 11βHSD2 is an NAD-dependent dehydrogenase, catalyzing the inactivation of cortisol to cortisone (Albiston et al. (1994) Mol. Cell. Endocrin. 105: R11-R17). The activity of these enzymes has profound consequences on glucocorticoid biology as evident by the fact that mutations in either gene cause human pathology. For example, 11βHSD2 is expressed in aldosterone-sensitive tissues such as the distal nephron, salivary gland, and colonic mucosa where its cortisol dehydrogenase activity serves to protect the intrinsically non-selective mineralcorticoid receptor from illicit occupation by cortisol (Edwards et al. (1988) Lancet 2: 986-989). Individuals with mutations in 11βHSD2 are deficient in this cortisol-inactivation activity and, as a result, present with a syndrome of apparent mineralcorticoid excess (also referred to as ‘SAME’) characterized by hypertension, hypokalemia, and sodium retention (Wilson et al. (1998) Proc. Natl. Acad. Sci. 95: 10200-10205). Likewise, mutations in 11βHSD1 and a co-localized NADPH-generating enzyme, hexose 6-phosphate dehydrogenase (H6PD), can result in cortisone reductase deficiency (also known as CRD; Draper et al. (2003) Nat. Genet. 34: 434-439). CRD patients excrete virtually all glucocorticoids as cortisone metabolites (tetrahydrocortisone) with low or absent cortisol metabolites (tetrahydrocortisols). When challenged with oral cortisone, CRD patients exhibit abnormally low plasma cortisol concentrations. These individuals present with ACTH-mediated androgen excess (hirsutism, menstrual irregularity, hyperandrogenism), a phenotype resembling polycystic ovary syndrome (PCOS).

Given the ability of 11βHSD1 to regenerate cortisol from inert circulating cortisone, considerable attention has been given to its role in the amplification of glucocorticoid function. 11βHSD1 is expressed in many key GR-rich tissues, including tissues of considerable metabolic importance such as liver, adipose, and skeletal muscle, and, as such, has been postulated to aid in the tissue-specific potentiation of glucocorticoid-mediated antagonism of insulin function. Considering a) the phenotypic similarity between glucocorticoid excess (Cushing's syndrome) and the metabolic syndrome with normal circulating glucocorticoids in the later, as well as b) the ability of 11βHSD1 to generate active cortisol from inactive cortisone in a tissue-specific manner, it has been suggested that central obesity and the associated metabolic complications in syndrome X result from increased activity of 11βHSD1 within adipose tissue, resulting in ‘Cushing's disease of the omentum’ (Bujalska et al. (1997) Lancet 349: 1210-1213). Indeed, 11βHSD1 has been shown to be upregulated in adipose tissue of obese rodents and humans (Livingstone et al. (2000) Endocrinology 131: 560-563; Rask et al. (2001) J. Clin. Endocrinol. Metab. 86: 1418-1421; Lindsay et al. (2003) J. Clin. Endocrinol. Metab. 88: 2738-2744; Wake et al. (2003) J. Clin. Endocrinol. Metab. 88: 3983-3988).

Additional support for this notion has come from studies in mouse transgenic models. Adipose-specific overexpression of 11βHSD1 under the control of the aP2 promoter in mouse produces a phenotype remarkably reminiscent of human metabolic syndrome (Masuzaki et al. (2001) Science 294: 2166-2170; Masuzaki et al. (2003) J. Clinical Invest. 112: 83-90). Importantly, this phenotype occurs without an increase in total circulating corticosterone, but rather is driven by a local production of corticosterone within the adipose depots. The increased activity of 11βHSD1 in these mice (2-3 fold) is very similar to that observed in human obesity (Rask et al. (2001) J. Clin. Endocrinol. Metab. 86: 1418-1421). This suggests that local l1 1HSD1-mediated conversion of inert glucocorticoid to active glucocorticoid can have profound influences whole body insulin sensitivity.

Based on this data, it would be predicted that the loss of 11βHSD 1 would lead to an increase in insulin sensitivity and glucose tolerance due to a tissue-specific deficiency in active glucocorticoid levels. This is, in fact, the case as shown in studies with 11βHSD1-deficient mice produced by homologous recombination (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). These mice are completely devoid of 11-keto reductase activity, confirming that 11βHSD1 encodes the only activity capable of generating active corticosterone from inert 11-dehydrocorticosterone. 11βHSD1-deficient mice are resistant to diet- and stress-induced hyperglycemia, exhibit attenuated induction of hepatic gluconeogenic enzymes (PEPCK, G6P), show increased insulin sensitivity within adipose, and have an improved lipid profile (decreased triglycerides and increased cardio-protective HDL). Additionally, these animals show resistance to high fat diet-induced obesity. Further, adipose-tissue overexpression of the 11-beta dehydrogenase enzyme, 11bHSD2, which inactivates intracellular corticosterone to 11-dehydrocorticosterone, similarly attenuates weight gain on high fat diet, improves glucose tolerance, and heightens insulin sensitivity. Taken together, these transgenic mouse studies confirm a role for local reactivation of glucocorticoids in controlling hepatic and peripheral insulin sensitivity, -and suggest that inhibition of 11βHSD1 activity may prove beneficial in treating a number of glucocorticoid-related disorders, including obesity, insulin resistance, hyperglycemia, and hyperlipidemia.

Data in support of this hypothesis has been published. Recently, it was reported that 11βHSD1 plays a role in the pathogenesis of central obesity and the appearance of the metabolic syndrome in humans. Increased expression of the 11βHSD1 gene is associated with metabolic abnormalities in obese women and that increased expression of this gene is suspected to contribute to the increased local conversion of cortisone to cortisol in adipose tissue of obese individuals (Engeli, et al., (2004) Obes. Res. 12: 9-17).

A new class of 11βHSD1 inhibitors, the arylsulfonamidothiazoles, was shown to improve hepatic insulin sensitivity and reduce blood glucose levels in hyperglycemic strains of mice (Barf et al. (2002) J. Med. Chem. 45: 3813-3815; Alberts et al. Endocrinology (2003) 144: 4755-4762). Additionally, it was recently reported that these selective inhibitors of 11βHSD1 can ameliorate severe hyperglycemia in genetically diabetic obese mice. Data using a structurally distinct series of compounds, the adamantyl triazoles (Hermanowski-Vosatka et al. (2005) J. Exp. Med. 202: 517-527), also indicates efficacy in rodent models of insulin resistance and diabetes, and further illustrates efficacy in a mouse model of atherosclerosis, perhaps suggesting local effects of corticosterone in the rodent vessel wall. Thus, 11βHSD1 is a promising pharmaceutical target for the treatment of the Metabolic Syndrome (Masuzaki, et al., (2003) Curr. Drug Targets Immune Endocr. Metabol. Disord. 3: 255-62).

A. Obesity and Metabolic Syndrome

As described above, multiple lines of evidence suggest that inhibition of 11βHSD1 activity can be effective in combating obesity and/or aspects of the metabolic syndrome cluster, including glucose intolerance, insulin resistance, hyperglycemia, hypertension, hyperlipidemia, and/or atherosclerosis/coronary heart disease. Glucocorticoids are known antagonists of insulin action, and reductions in local glucocorticoid levels by inhibition of intracellular cortisone to cortisol conversion should increase hepatic and/or peripheral insulin sensitivity and potentially reduce visceral adiposity. As described above, 11βHSD1 knockout mice are resistant to hyperglycemia, exhibit attenuated induction of key hepatic gluconeogenic enzymes, show markedly increased insulin sensitivity within adipose, and have an improved lipid profile. Additionally, these animals show resistance to high fat diet-induced obesity (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). In vivo pharmacology studies with multiple chemical scaffolds have confirmed the critical role for 11βHSD1 in regulating insulin resistance, glucose intolerance, dyslipidemia, hypertension, and atherosclerosis. Thus, inhibition of 11βHSD1 is predicted to have multiple beneficial effects in the liver, adipose, skeletal muscle, and heart, particularly related to alleviation of component(s) of the metabolic syndrome, obesity, and/or coronary heart disease.

B. Pancreatic Function

Glucocorticoids are known to inhibit the glucose-stimulated secretion of insulin from pancreatic beta-cells (Billaudel and Sutter (1979) Horm. Metab. Res. I1: 555-560). In both Cushing's syndrome and diabetic Zucker fa/fa rats, glucose-stimulated insulin secretion is markedly reduced (Ogawa et al. (1992) J. Clin. Invest. 90: 497-504). 11βHSD1 mRNA and activity has been reported in the pancreatic islet cells of ob/ob mice and inhibition of this activity with carbenoxolone, an 11βHSD1 inhibitor, improves glucose-stimulated insulin release (Davani et al. (2000) J. Biol. Chem. 275: 34841-34844). Thus, inhibition of 11βHSD1 is predicted to have beneficial effects on the pancreas, including the enhancement of glucose-stimulated insulin release and the potential for attenuating pancreatic beta-cell decompensation.

C. Cognition and Dementia

Mild cognitive impairment is a common feature of aging that may be ultimately related to the progression of dementia. In both aged animals and humans, inter-individual differences in general cognitive function have been linked to variability in the long-term exposure to glucocorticoids (Lupien et al. (1998) Nat. Neurosci. 1: 69-73). Further, dysregulation of the HPA axis resulting in chronic exposure to glucocorticoid excess in certain brain subregions has been proposed to contribute to the decline of cognitive function (McEwen and Sapolsky (1995) Curr. Opin. Neurobiol. 5: 205-216). 11HSD1 is abundant in the brain, and is expressed in multiple subregions including the hippocampus, frontal cortex, and cerebellum (Sandeep et al. (2004) Proc. Natl. Acad. Sci. Early Edition: 1-6). Treatment of primary hippocampal cells with the 11βHSD1 inhibitor carbenoxolone protects the cells from glucocorticoid-mediated exacerbation of excitatory amino acid neurotoxicity (Rajan et al. (1996) J. Neurosci. 16: 65-70). Additionally, 11βHSD1-deficient mice are protected from glucocorticoid-associated hippocampal dysfunction that is associated with aging (Yau et al. (2001) Proc. Natl. Acad. Sci. 98: 4716-4721). In two randomized, double-blind, placebo-controlled crossover studies, administration of carbenoxolone improved verbal fluency and verbal memory (Sandeep et al. (2004) Proc. Natl. Acad. Sci. Early Edition: 1-6). Thus, inhibition of 11βHSD1 is predicted to reduce exposure to glucocorticoids in the brain and protect against deleterious glucocorticoid effects on neuronal function, including cognitive impairment, dementia, and/or depression.

D. Intra-Ocular Pressure

Glucocorticoids can be used topically and systemically for a wide range of conditions in clinical ophthalmology. One particular complication with these treatment regimens is corticosteroid-induced glaucoma. This pathology is characterized by a significant increase in intra-ocular pressure (IOP). In its most advanced and untreated form, IOP can lead to partial visual field loss and eventually blindness. IOP is produced by the relationship between aqueous humour production and drainage. Aqueous humour production occurs in the non-pigmented epithelial cells (NPE) and its drainage is through the cells of the trabecular meshwork. 11βHSD1 has been localized to NPE cells (Stokes et al. (2000) Invest. Ophthalmol. Vis. Sci. 41: 1629-1683; Rauz et al. (2001) Invest. Ophthalmol. Vis. Sci. 42: 2037-2042) and its function is likely relevant to the amplification of glucocorticoid activity within these cells. This notion has been confirmed by the observation that free cortisol concentration greatly exceeds that of cortisone in the aqueous humour (14:1 ratio). The functional significance of 11βHSD1 in the eye has been evaluated using the inhibitor carbenoxolone in healthy volunteers (Rauz et al. (2001) Invest. Ophthalmol. Vis. Sci. 42: 2037-2042). After seven days of carbenoxolone treatment, IOP was reduced by 18%. Thus, inhibition of 11βHSD1 in the eye is predicted to reduce local glucocorticoid concentrations and IOP, producing beneficial effects in the management of glaucoma and other visual disorders.

E. Hypertension

Adipocyte-derived hypertensive substances such as leptin and angiotensinogen have been proposed to be involved in the pathogenesis of obesity-related hypertension (Matsuzawa et al. (1999) Ann. N.Y. Acad. Sci. 892: 146-154; Wajchenberg (2000) Endocr. Rev. 21: 697-738). Leptin, which is secreted in excess in aP2-11βHSD1 transgenic mice (Masuzaki et al. (2003) J. Clinical Invest. 112: 83-90), can activate various sympathetic nervous system pathways, including those that regulate blood pressure (Matsuzawa et al. (1999) Ann. N.Y. Acad. Sci. 892: 146-154). Additionally, the renin-angiotensin system (RAS) has been shown to be a major determinant of blood pressure (Walker et al. (1979) Hypertension 1: 287-291). Angiotensinogen, which is produced in liver and adipose tissue, is the key substrate for renin and drives RAS activation. Plasma angiotensinogen levels are markedly elevated in aP2-11βHSD1 transgenic mice, as are angiotensin II and aldosterone (Masuzaki et al. (2003) J. Clinical Invest. 112: 83-90). These forces likely drive the elevated blood pressure observed in aP2-11HSD1 transgenic mice. Treatment of these mice with low doses of an angiotensin II receptor antagonist abolishes this hypertension (Masuzaki et al. (2003) J. Clinical Invest. 112: 83-90). This data illustrates the importance of local glucocorticoid reactivation in adipose tissue and liver, and suggests that hypertension may be caused or exacerbated by 11βHSD1 activity. Thus, inhibition of 11βHSD1 and reduction in adipose and/or hepatic glucocorticoid levels is predicted to have beneficial effects on hypertension and hypertension-related cardiovascular disorders.

F. Bone Disease

Gluccorticoids can have adverse effects on skeletal tissues. Continued exposure to even moderate glucocorticoid doses can result in osteoporosis (Cannalis (1996) J. Clin. Endocrinol. Metab. 81: 3441-3447) and increased risk for fractures. Experiments in vitro confirm the deleterious effects of glucocorticoids on both bone-resorbing cells (also known as osteoclasts) and bone forming cells (osteoblasts). 11βHSD1 has been shown to be present in cultures of human primary osteoblasts as well as cells from adult bone, likely a mixture of osteoclasts and osteoblasts (Cooper et al. (2000) Bone 27: 375-381), and the 11βHSD1 inhibitor carbenoxolone has been shown to attenuate the negative effects of glucocorticoids on bone nodule formation (Bellows et al. (1998) Bone 23: 119-125). Thus, inhibition of 11βHSD1 is predicted to decrease the local glucocorticoid concentration within osteoblasts and osteoclasts, producing beneficial effects in various forms of bone disease, including osteoporosis.

Small molecule inhibitors of 11βHSD1 are currently being developed to treat or prevent 11βHSD1-related diseases such as those described above. For example, certain amide-based inhibitors are reported in WO 2004/089470, WO 2004/089896, WO 2004/056745, and WO 2004/065351. Antagonists of 11βHSD1 have been evaluated in human clinical trials (Kurukulasuriya, et al., (2003) Curr. Med. Chem. 10: 123-53).

In light of the experimental data indicating a role for 11βHSD1 in glucocorticoid-related disorders, metabolic syndrome, hypertension, obesity, insulin resistance, hyperglycemia, hyperlipidemia, type 2 diabetes, atherosclerosis, androgen excess (hirsutism, menstrual irregularity, hyperandrogenism), polycystic ovary syndrome (PCOS), and other diseases, therapeutic agents aimed at augmentation or suppression of these metabolic pathways, by modulating glucocorticoid signal transduction at the level of 11βHSD1 are desirable.

Furthermore, because the MR binds to aldosterone (its natural ligand) and cortisol with equal affinities, compounds that are designed to interact with the active site of 11βHSD1 (which binds to cortisone/cortisol) may also interact with the MR and act as antagonists. Because the MR is implicated in heart failure, hypertension, and related pathologies including atherosclerosis, arteriosclerosis, coronary artery disease, thrombosis, angina, peripheral vascular disease, vascular wall damage, and stroke, MR antagonists are desirable and may also be useful in treating complex cardiovascular, renal, and inflammatory pathologies including disorders of lipid metabolism including dyslipidemia or hyperlipoproteinaemia, diabetic dyslipidemia, mixed dyslipidemia, hypercholesterolemia, hypertriglyceridemia, as well as those associated with type 1 diabetes, type 2 diabetes, obesity, metabolic syndrome, and insulin resistance, and general aldosterone-related target-organ damage.

As evidenced herein, there is a continuing need for new and improved drugs that target 11βHSD1. The compounds, compositions and methods described herein help meet this and other needs.

### SUMMARY OF THE INVENTION

The present invention provides, inter alia, compounds of Formula Ia or Ib: 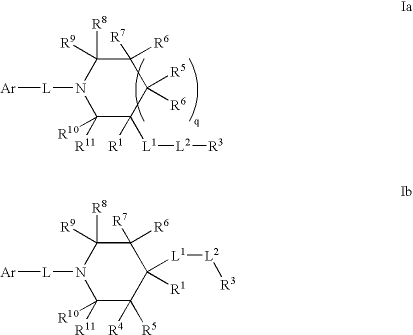
or pharmaceutically acceptable salts or prodrugs thereof, wherein constituent members are defined herein.

The present invention further provides methods of modulating 11βHSD1 by contacting 11βHSD1 with a compound of the invention.

The present invention further provides methods of inhibiting 11βHSD1 by contacting 11βHSD1 with a compound of the invention.

The present invention further provides methods of inhibiting the conversion of cortisone to cortisol in a cell by contacting the cell with a compound of the invention.

The present invention further provides methods of inhibiting the production of cortisol in a cell by contacting the cell with a compound of the invention.

The present invention further provides methods of treating diseases associated with activity or expression of 11βHDS1.

### DETAILED DESCRIPTION

The present invention provides, inter alia, a compound of Formula Ia or Ib: 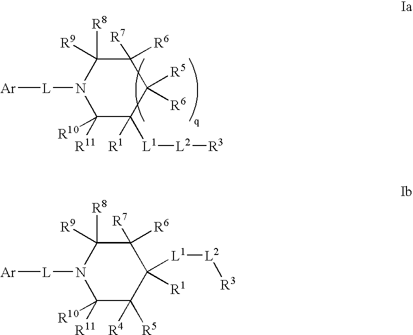
or pharmaceutically acceptable salt or prodrug thereof, wherein:

L is absent, S(O)₂, S(O), S, S(O)₂NR², C(O), C(O)O, C(O)O—(C₁₋₃ alkylene), or C(O)NR²;

L¹ is O, CH₂, or NR^{N};

L² is CO or S(O)₂;

provided that when L¹ is NR^{N}, L² is SO2;

R^{N} is H, C₁₋₆ alkyl, aryl, cycloalkyl, heteroaryl or heterocycloalkyl;

Ar is aryl or heteroaryl, each optionally substituted by 1, 2, 3, 4 or 5 —W—X—Y-Z;

R¹ is H, C(O)OR^{b}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹⁴;

R is H or C₁₋₆ alkyl;

R³is H, C₁₋₆ alkyl, aryl, cycloalkyl, heteroaryl or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;

or R³is NR^{3a}R^{3b} or OR^{3c};

R^{3a} and R^{3b} are independently selected from H, C₁₋₆ alkyl, aryl, cycloalkyl, heteroaryl and heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;

or R^{3a} and R^{3b} together with the N atom to which they are attached form a 4-14 membered heterocycloalkyl group which is optionally substituted by 1, 2 or 3 —W′—X′—Y′-Z′;

R^{3c} is H, C₁₋₆ alkyl, aryl, cycloalkyl, heteroaryl or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;

R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, hetero heterocycloalkyl, arylalkyl, heteroarylalkyl, cycloalkylalkyl and heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹⁴;

or R¹ and R³ together with the carbon atoms to which they are attached and the intervening —NR²CO— moiety form a 4-14 membered heterocycloalkyl group which is optionally substituted by 1, 2 or 3 R¹⁴;

or R⁴ and R⁵ together with the carbon atom to which they are attached form a 3-14 membered cycloalkyl or heterocycloalkyl group which is optionally substituted by 1, 2 or 3 R¹⁴;

or R⁶ and R⁷ together with the carbon atom to which they are attached form a 3-14 membered cycloalkyl or heterocycloalkyl group which is optionally substituted by 1, 2 or 3 R¹⁴;

or R⁸ and R⁹ together with the carbon atom to which they are attached form a 3-14 membered cycloalkyl or heterocycloalkyl group which is optionally substituted by 1, 2 or 3 R¹⁴;

or R¹⁰ and R¹¹ together with the carbon atom to which they are attached form a 3-14 membered cycloalkyl or heterocycloalkyl group which is optionally substituted by 1, 2 or 3 R¹⁴;

or R⁴ and R⁶ together with the carbon atom to which they are attached form a 3-7 membered fused cycloalkyl group or 3-7 membered fused heterocycloalkyl group which is optionally substituted by 1,2 or 3 R¹⁴;

or R⁶ and R⁸ together with the carbon atom to which they are attached form a 3-7 membered fused cycloalkyl group or 3-7 membered fused heterocycloalkyl group which is optionally substituted by 1, 2or3R¹⁴;

each R¹⁴ is independently halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}. NR^{c′}R^{d′}, NR^{c′}C(O)R^{d′}, NR^{c′}C(O)OR_{a′}, NR^{c′}S(O)₂R^{b′}, S(O)R^{b′}, S(O)NR^{c′}R^{d′}, S(O)₂R^{b′}, or S(O)₂NR^{c′}R^{d′};

W, W′ and W″ are independently selected from absent, C₁₋₁₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of said C₁₋₆ alkylenyl, C₂₋₆ alkenylenyl and C₂₋₆ alkynylenyl is optionally substituted by 1, 2 or 3 independently selected from halo, OH, C₁₋₄ alkoxy, C₁₋₄ haloalkoxy, amino, C₁₋₄ alkylamino and C₂₋₈ dialkylamino;

X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of said C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₄ alkyl, C₁₋₄ haloalkyl, C₂₋₈ alkoxyalkyl, C₁₋₄ alkoxy, C₁₋₄ haloalkoxy, C₂₋₈ alkoxyalkoxy, cycloalkyl, heterocycloalkyl, C(O)OR^{a}, C(O)NR^{c}R^{d}, amino, C₁₋₄ alkylamino and C₂₋₈ dialkylamino;

Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl and C₂₋₆ alkynylenyl is optionally substituted by 1, 2 or 3 substituents independently selected from halo, OH, C₁₋₄ alkoxy, C₁₋₄ haloalkoxy, amino, C 1₄ alkylamino and C₂₋₈ dialkylamino;

Z, Z′ and Z″ are independently selected from H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of said C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, and S(O)₂NR^{c}R^{d};

wherein two —W—X—Y-Z attached to the same atom optionally form a 3-14 membered cycloalkylk or 3-14 membered heterocycloalkyl group optionally substituted by 1, 2 or 3 —W′—X″—Y″-Z″;

wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;

wherein —W—X—Y-Z is other than H;

wherein —W′—X′—Y′-Z′ is other than H;

wherein —W″—X″—Y″-Z″ is other than H;

R^{a} and R^{a ′} are independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of said C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;

R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of said C₁₋₆ alkyl, C₁₋₆ haloalkyl, C₂₋₆ alkenyl, C₂₋₆ alkynyl, a cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c′} and R^{d′} together with the N atom to which they are attached form a 4-, 5-, 6- or 7-membered heterocycloalkyl group;

R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{e} and R^{f} together with the N atom to which they are attached form a 4-, 5-, 6- or 7-membered heterocycloalkyl group;

R^{g} is H, CN, NO₂, C(O)NH₂, or C₁₋₆ alkyl; and

q is 0, 1 or 2.

In some embodiments, when the compound has Formula Ia; q is 1; L is C(O)CH₂; L¹ is CH₂; L² is S(O)₂; R⁴, R⁵, R⁶, R⁷, R⁸, R⁹, R¹⁰ and R¹¹ are each H; R³ is NR^{3a}R^{3b}; and R^{3a} and R^{3b} together with the N atom to which they are attached form an optionally substituted 4-14 membered heterocycloalkyl group, then R³ is other than piperidinyl substituted by heteroaryl wherein the heteroaryl is optionally substituted by arylalkyl.

In some embodiments, when the compound has Formula Ia, q is 0, L is C(O)CH₂, R³ is NR^{3a}R^{3b}, and R^{3a} and R^{3b} together with the N atom to which they are attached form an optionally substituted 4-14 membered heterocycloalkyl group, then Ar is other than optionally substituted aryl.

In some embodiments, when the compound has Formula Ia, q is 0, L is CO or S(O)₂, R³ is NR^{3a}R^{3b}, and R^{3a} and R^{3b} together with the N atom to which they are attached form an optionally substituted 4-14 membered heterocycloalkyl group, then each of R⁴, R⁵, R⁶, R⁷, R⁸, R⁹, R¹⁰ and R¹¹ is other than OC(O)R^{a′}, OC(O)OR^{b′}, C(O)OR^{b′} or OC(O)NR^{c′}R^{d′}.

In some embodiments, when the compound has Formula Ia, q is 0, L is absent, R³ is NR^{3a}R^{3b}, and R^{3a} and R^{3b} together with the N atom to which they are attached form an optionally substituted 4-14 membered heterocycloalkyl group, then R³ is other than optionally substituted piperazinyl or optionally substituted 3-oxo-piperazinyl.

In some embodiments, L is S(O)₂.

In some embodiments, L is absent.

In some embodiments, L is CO.

In some embodiments, L¹ is O and L² is CO.

In some embodiments, L¹ is CH₂ and L² is CO.

In some embodiments, L¹ is CH₂ and L² is S(O)₂.

In some embodiments, L¹ is NH and L² is S(O)₂.

In some embodiments, L¹ is O and L² is S(O)₂.

In some embodiments, R^{N} is H or C₁₋₆ alkyl. In some further embodiments, R^{N} is H.

In some embodiments, R¹ is H, C₁₁₋₀ alkyl, C₁₋₁₀ haloalkyl, C_{2 10} alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

In some embodiments, R¹ is H, C₁₋₆ alkyl, or C₁₋₆ haloalkyl.

In some embodiments, R³ is NR^{3a}R^{3b}; R^{3a} is H or C₁₋₆ alkyl; and R^{3b} is a 4-14 membered heterocycloalkyl group which is optionally substituted by 1, 2 or 3 —W′—X′—Y′-Z′.

In some embodiments, R³ is NR^{3a}R^{3b}; R^{3a} is C₁₋₆ alkyl; and R^{3b} is a 4-7 membered heterocycloalkyl group which is optionally substituted by 1, 2 or 3 —W′—X′—Y′-Z′.

In some embodiments, R³ is NR^{3a}R^{3b}; R³a is C₁I₆ alkyl; and R³b is a 4-7 membered heterocycloalkyl group.

In some embodiments, R³ is NR^{3a}R^{3b} and R^{3a} and R^{3b} together with the N atom to which they are attached form a 4-14 membered heterocycloalkyl group which is optionally substituted by 1, 2 or 3 —W′—X′—Y′-Z′.

In some embodiments, R⁴, R⁵, R⁶, R⁷, R⁸, R⁹, R¹⁰ and R¹¹ are independently selected from H, NR^{c′}R^{d′}, NR^{c′}C(O)R^{a′}NR^{c′}C(O)OR^{b′},S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl.

In some embodiments, R⁴, R⁵, R⁶, R⁷, R⁸, R⁹, R¹⁰ and R¹¹ are independently selected from H, C₁₋₆ alkyl, C₁₋₆ haloalkyl, C₂₋₆ alkenyl, C₂₋₆ alkynyl, aryl, cycloalkyl, heteroaryl, heterocycloalky 20 arylalkyl, heteroarylalkyl, cycloalkylalkyl and heterocycloalkylalkyl.

In some embodiments, R⁴, R⁵, R⁶, R⁷, R⁸, R⁹, R¹⁰ and R¹¹ are independently selected from H, C₁₋₆ alkyl, C₁₋₆ haloalkyl, C₂₋₆ alkenyl and C₂₋₆ alkynyl.

In some embodiments, R⁴, R⁵, R⁶, R⁷, R⁸, R⁹, R¹⁰ and R¹¹ are independently selected from H, C₁₋₆ alkyl and C₁₋₆ haloalkyl.

In some embodiments, each R¹⁴ is independently halo, C₁₋₄ alkyl, C₁₋₄ haloalkyl, aryl, cycloalkyl, heteroaryl, heterocycloalkyl, CN, NO₂, OR^{a′} or SR^{a′}.

In some embodiments, each R¹⁴ is independently halo, C₁₋₄ alkyl, C₁₋₄ haloalkyl, CN, NO₂, OH, —OC₁₋₄ alkyl, or —SC₁₋₄ alkyl.

In some embodiments, q is 0 or 1. In some further embodiments, q is 1.

In some embodiments, the compounds of the invention have Formula II: 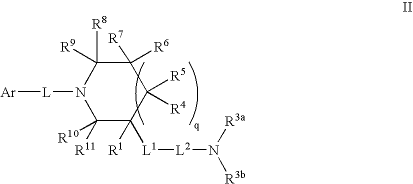
wherein R^{3a} and R^{3b} together with the N atom to which they are attached form a 4-14 membered heterocycloalkyl group which is optionally substituted by 1, 2 or 3 —W′—X′—Y′-Z′.

In some embodiments of the compounds of Formula II, the ring-forming atoms of the heterocycloalkyl group are selected from N, C and O.

In some embodiments of the compounds of Formula II, L is absent, S(O)₂ or CO.

In some embodiments of the compounds of Formula II, q is 0 or 1. In some further embodiments, q is 1.

In some embodiments, the compounds of the invention have Formula III: 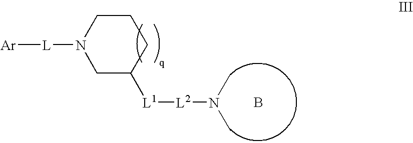
wherein ring B is a 4-14 membered heterocycloalkyl group which is optionally substituted by 1, 2 or 3—W′—X′—Y′-Z′.

In some embodiments of the compounds of Formula III, L is absent, S(O)₂ or CO.

In some embodiments of the compounds of Formula III, the compound has Formula IVa, IVb, IVc, or IVd:

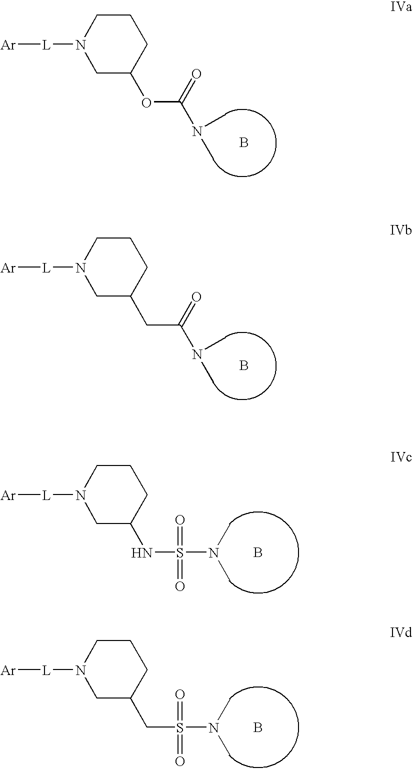

In some embodiments, the ring-forming atoms of ring B are selected from N, C and O.

In some embodiments, ring B is pyrrolidinyl, piperidinyl, morpholino, 8-azabicyclo[3.2.1]octan-8-yl, 9-azabicyclo[3.3.1]nonan-9-yl or 2-oxa-6-azatricyclo[3.3.1.1(3,7)]decan-6-yl, each optionally substituted by 1, 2 or 3 —W′—X′—Y′-Z′.

In some embodiments, ring B is substituted by 1 OH.

In some embodiments, the compounds of the invention have Formula IVa or Formula IVb. In some further embodiments, the compounds of the invention have Formula IVa.

In some embodiments, Ar is aryl optionally substituted by 1, 2, 3, 4 or 5 —W—X—Y-Z.

In some embodiments, Ar is phenyl or naphthyl, each optionally substituted by 1, 2, 3, 4 or 5 —W—X—Y-Z.

In some embodiments, Ar is phenyl or naphthyl, each optionally substituted by 1, 2, 3, 4 or 5 substituents independently selected from halo, CN, NO₂, C₁₋₄ alkoxy, heteroaryloxy, C₂₋₆ alkynyl, C₁₋₄ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected form halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

In some embodiments, Ar is phenyl or naphthyl, each optionally substituted by 1, 2, 3, 4 or 5 substituents independently selected from halo, CN, NO₂, NR^{c}C(O)R^{d}, NR^{c}C(O)OR^{a}, NR^{c}R^{d}, C₁₋₆ alkyl, aryl and heteroaryl, wherein each of said aryl and heteroaryl is optionally substituted by 1, 2 or 3 substituents independently selected from C₁₋₆ alkyl and C(O)NR^{c}R^{d}.

In some embodiments, Ar is heteroaryl optionally substituted by 1, 2, 3, 4 or 5 —W—X—Y-Z.

In some embodiments, Ar is heteroaryl optionally substituted by 1, 2, 3, 4 or 5 substituents independently selected from halo, CN, NO₂, C₁₋₄ alkoxy, heteroaryloxy, C₂₋₆ alkynyl, C₁₋₄ haloalkoxy, NR^{c}C(O)R^{d}, NR^{c}C(O)OR^{a}, C(O)NR^{c}R^{d}, NR^{c}R^{d}, NR^{c}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected from halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

In some embodiments, Ar is pyridyl, pyrimidinyl, thienyl, thiazolyl, quinolinyl, 2,1,3-benzoxadiazolyl, isoquinolinyl or isoxazolyl, each optionally substituted by 1, 2, 3, 4 or 5 substituents independently selected from halo, CN, NO₂, C₁₋₄ alkoxy, heteroaryloxy, C₂₋₆ alkynyl, C₁₋₄ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected from halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

In some embodiments, Ar is pyridyl optionally substituted by 1, 2, 3, 4 or 5 substituents independently selected from halo, CN, NO₂, C₁₋₄ alkoxy, heteroaryloxy, C2 6 alkynyl, C₁₋₄ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected from halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

In some embodiments, the compounds of the invention have Formula Va, Vb or Vc: 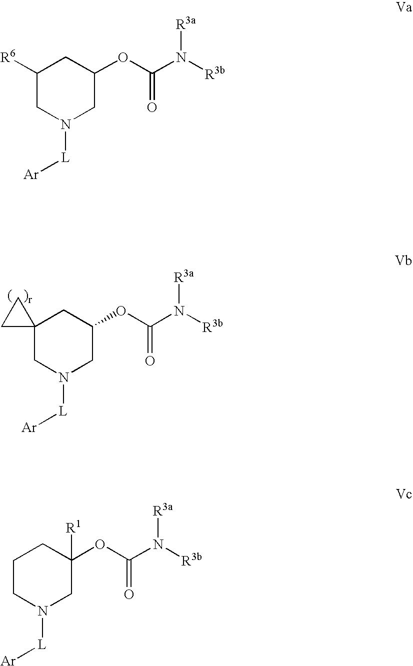
wherein:

r is 1, 2, 3, 4 or 5; and

R^{3a} and R^{3b} together with the N atom to which they are attached form a 4-14 membered heterocycloalkyl group which is optionally substituted by 1, 2 or 3 —W′—X′—Y′-Z′.

In some embodiments, the compounds of the invention have Formula Ia; L¹ is 0; L² is CO; q is 1; R³ is NR^{3a}R^{3b}; R^{3a} is C₁₋₆ alkyl; and R^{3b} is a 4-7 membered heterocycloalkyl group.

In some embodiments, each —W—X—Y-Z is independently selected from halo, nitro, cyano, OH, C₁₋₄ alkyl, C₁₋₄ haloalkyl, C₁₋₄ haloalkoxy, amino, C₁₋₄ alkoxy, cycloalkylcarbonylamino, alkoxycarbonylamino, alkylsulfonylamino, cycloalkylalkylcarbonylamino, acyl(alkyl)amino, alkylamino, dialkylamino, dialkylaminosulfonyl, dialkylaminocarbonyl, dialkylaminocarbonylalkyloxy, alkylcarbonyl(alkyl)amino, cycloalkylcarbonyl(alkyl)amino, alkoxycarbonyl(alkyl)amino, alkoxycarbonyl, alkylsulfonyl, arylsulfonyl, aryl, cycloalkyl, heteroaryl, heterocycloalkyl, aryloxy, cycloalkyloxy, heteroaryloxy, heterocycloalkyloxy, arylalkyloxy, and acylamino;

wherein each of said aryl, cycloalkyl, heteroaryl, heterocycloalkyl, arylalkyloxy and heterocycloalkyloxy is optionally substituted by 1 or more substituents independently selected from halo, C₁₋₄ alkyl, OH, C₁₋₄ alkoxy, cycloalkylaminocarbonyl, alkoxycarbonyl, cyano, acyl, acylamino, alkylsulfonyl, amino, alkylamino, dialkylamino, and aminocarbonyl.

In some embodiments, each —W—X—Y-Z is independently selected from halo, CN, NO₂, C₁₋₄ alkoxy, heteroaryloxy, C₂₋₆ alkynyl, C₁₋₄ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected from halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

In some embodiments, each —W′—X′—Y′-Z′ is independently selected from halo, OH, cyano, nitro, C₁₋₄ alkyl, C₁₋₄ alkoxy, C₁₋₄ haloalkyl, C₁₋₄ haloalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, and arylsulfonyl;

wherein each of said aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl and heterocycloalkyloxy is optionally substituted by 1 or 2 substituents independently selected from halo, OH, cyano, nitro, C₁₋₄ alkyl, C₁₋₄ alkoxy, C₁₋₄ haloalkyl, C₁-4 haloalkoxy, amino, alkylamino, dialkylamino, hydroxyalkyl, and alkoxycarbonyl.

In some embodiments, each —W′—X′—Y′-Z′ is independently selected from halo, OH, cyano, nitro, C₁₋₄ alkyl, C₁₋₄ alkoxy, C₁₋₄ haloalkyl, C₁₋₄ haloalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, and arylsulfonyl.

In some embodiments, each —W″—X″—Y″-Z″ is independenly selected from halo, OH, cyano, nitro, C₁₋₄ alkyl, C₁₋₄ alkoxy, C₁₋₄ haloalkyl, C₁₋₄ haloalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, and arylsulfonyl.

In some embodiments, Z, Z′ and Z″ are independently selected from H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of said C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, and S(O)₂NR^{c}R^{d}.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkylene” refers to a divalent alkyl linking group.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “haloalkyl” refers to an alkyl group having one or more halogen substituents. Example haloalkyl groups include CF₃, C₂F₅, CHF₂, CCl₃, CHCl₂, C₂Cl₅, CH₂CF₃, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of cyclopentane, cyclopentene, cyclohexane, and the like.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. A ring forming N atom can be optionally substituted with oxo. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or I to 2 heteroatoms.

As used herein, “heterocycloalkyl” refers to non-aromatic heterocycles where one or more of the ring-forming atoms is a heteroatom such as an O, N, or S. Hetercycloalkyl groups can be mono or polycyclic (e.g., both fused and spiro systems). Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by one or more oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkoxy” refers to an —O-haloalkyl group. An example haloalkoxy group is OCF₃.

As used herein, “alkoxyalkyl” refers to an alkyl group substituted by an alkoxy group. One example of alkoxyalkyl is —CH₂—OCH₃.

As used herein, “alkoxyalkoxy” refers to an alkoxy group substituted by an alkoxy group. One example of alkoxyalkoxy is —OCH₂CH₂—OCH₃.

As used herein, “arylalkyl” refers to alkyl substituted by aryl and “cycloalkylalkyl” refers to alkyl substituted by cycloalkyl. An example arylalkyl group is benzyl.

As used herein, “heteroarylalkyl” refers to an alkyl group substituted by a heteroaryl group.

As used herein, “amino” refers to NH₂.

As used herein, “alkylamino” refers to an amino group substituted by an alkyl group.

As used herein, “dialkylamino” refers to an amino group substituted by two alkyl groups.

As used herein, “dialkylaminocarbonyl” refers to a carbonyl group substituted by a dialkylamino group.

As used herein, “dialkylaminocarbonylalkyloxy” refers to an alkyloxy (alkoxy) group substituted by a carbonyl group which in turn is substituted by a dialkylamino group.

As used herein, “cycloalkylcarbonyl(alkyl)amino” refers to an alkylamino group substituted by a carbonyl group (on the N atom of the alkylamino group) which in turn is substituted by a cycloalkyl group. The term “cycloalkylcarbonylamino” refers to an amino group substituted by a carbonyl group (on the N atom of the amino group) which in turn is substituted by a cycloalkyl group. The term “cycloalkylalkylcarbonylamino” refers to an amino group substituted by a carbonyl group (on the N atom of the amino group) which in turn is substituted by a cycloalkylalkyl group.

As used herein, “alkoxycarbonyl(alkyl)amino” refers to an alkylamino group substituted by an alkoxycarbonyl group on the N atom of the alkylamino group. The term “alkoxycarbonylamino” refers to an amino group substituted by an alkoxycarbonyl group on the N atom of the amino group.

As used herein “alkoxycarbonyl” refers to a carbonyl group substituted by an alkoxy group.

As used herein, “alkylsulfonyl” refers to a sulfonyl group substituted by an alkyl group. The term “alkylsulfonylamino” refers to an amino group substituted by an alkylsulfonyl group.

As used herein, “arylsulfonyl” refers to a sulfonyl group substituted by an aryl group.

As used herein, “dialkylaminosulfonyl” refers to a sulfonyl group substituted by dialkylamino.

As used herein, “arylalkyloxy” refers to —O-arylalkly. An example of an arylalkyloxy group is benzyloxy.

As used herein, “cycloalkyloxy” refers to —O-cycloalkyl. An example of a cycloalkyloxy group is cyclopenyloxyl.

As used herein, “heterocycloalkyloxy” refers to —O-heterocycloalkyl.

As used herein, “heteroaryloxy” refers to —O-heteroaryl. An example is pyridyloxy.

As used herein, “acylamino” refers to an amino group substituted by an alkylcarbonyl (acyl) group. The term “acyl(alkyl)amino” refers to an amino group substituted by an alkylcarbonyl (acyl) group and an alkyl group.

As used herein, “alkylcarbonyl” refers to a carbonyl group substituted by an alkyl group.

As used herein, “cycloalkylaminocarbonyl” refers to a carbonyl group substituted by an amino group which in turn is substituted by a cycloalkyl group.

As used herein, “aminocarbonyl” refers to a carbonyl group substituted by an amino group (i.e., CONH₂).

As used herein, “hydroxyalkyl” refers to an alkyl group substituted by a hydroxyl group. An example is —CH₂OH.

As used herein, “alkylcarbonyloxy” refers to an oxy group substituted by a carbonyl group which in turn is substituted by an alkyl group [i.e., —O—C(O)-(alkyl)].

As used herein, “halosulfanyl” refers to a sulfur group having one or more halogen substituents. Example halosulfanyl groups include pentahalosulfanyl groups such as SF₅.

As used herein, the terms “substitute” or “substitution” refer to replacing a hydrogen with a non-hydrogen moiety.

The compounds described herein can be asymmetric (e.g., having one or more stereocenters). All stereoisomers, such as enantiomers and diastereomers, are intended unless otherwise indicated.

Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1 H- and 2H-isoindole, and 1 H- and 2H-pyrazole. Tautomeric forms can be in equilibrium or sterically locked into one form by appropriate substitution.

Compounds of the invention further include hydrates and solvates, as well as anhydrous and non-solvated forms.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which is was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The-processes described herein can be monitored according to any suitable method known in the art. For example, product formation can be monitored by spectroscopic means, such as nuclear magnetic resonance spectroscopy (e.g., ¹H or ¹³C NMR), infrared spectroscopy (IR), spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention can be prepared, for example, using the reaction pathways and techniques as described below.

A series of O-(piperidin-3-yl)carbamates of formula 1-5 can be prepared by the method described in Scheme 1. 1-(tert-Butoxycarbonyl)-3-hydroxy-piperidine 1-1 can be treated with p-nitrophenyl chloroformate or carbonyl diimidazole in the presence of a base such as triethylamine to provide an activated species such as p-nitrophenyl carbonic acid ester (i.e., cabornate) 1-2, or the corresponding imidazole carbamate. The activated species such as as p-nitrophenyl carbonic acid ester 1-2 can be reacted with an appropriate amine NHR^{3a}R^{3b} to give the desired carbamate 1-3. The Boc protecting group of the compound 1-3 can be removed under a suitable condition such as by treatment with HCl in 1,4-dioxane or by treatment with trifluoroacetic acid to afford the corresponding HCl salt 1-4 or the corresponding TFA salt, which can further be coupled with an appropriate chloride ArLCl to give the compound of formula 1-5. Also as shown in Scheme AB-1, compounds of formula A-1-5 and B-1-5 can be made by similar transformations to those described in Scheme 1 from the appropriate starting materials.

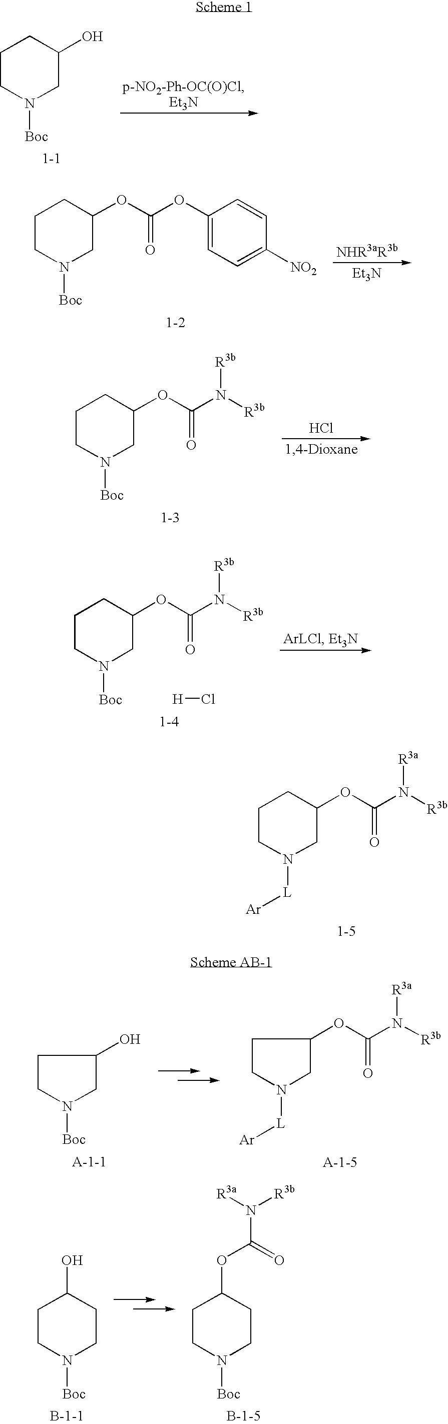

As shown in Scheme 2, alternatively, a series of O-(piperidin-3-yl)carbamates of formula 2-4 (same as formula 1-5 in Scheme 1) can be prepared in a similar fashion as described in Scheme 1 but with a change of the coupling sequences. Also as shown in Scheme AB-2, compounds of formula A-2-4 and B-2-4 can be made by similar transformations to those described in Scheme 2 from the appropriate starting materials.

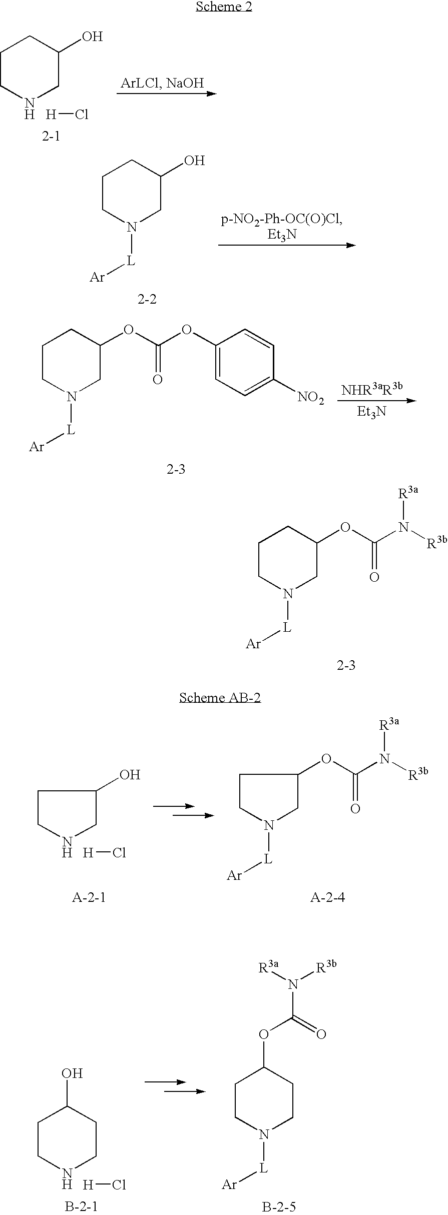

A series of carbamate compounds of formula 3-2 can be prepared by the method outlined in Scheme 3. Piperidin-3-ylcarbamate 3-1 can be coupled to an aryl halide or a heteroaryl halide ArX (wherein Ar can be aryl or heteroaryl, each of which is optionally substituted with one or more substituents such as halo or alkyl) such as bromobenzene in an organic solvent such as dimethyl sulfoxide, in the presence of a base such as tert-butoxide, to afford a compound of formula 3-2. When Ar is heteroaryl, the coupling can be achieved by heating 3-1 and the ArX in a suitable solvent such as N-methylpyrrolidinone in the presence of a suitable base such as diisopropylethylamine. Alternatively, carbamate compounds of formula 3-2 can be prepared by coupling of 3-1 to an optionally substituted aryl boronic acid or a heteroaryl boronic acid, catalyzed by copper acetate as described by Patrick Lam et al (J. Comb. Chem. 2002, 4, 179). Carbamate compounds 3-1 can also be coupled to an optionally substituted aryl halide or a heteroaryl halide ArX in the presence of copper iodide and ethylene glycol as described by Stephen Buchwald et al (Org. Lett. 2002, 4, 581); or in the presence of an appropriate palladium catalyst known to one skilled in the art of organic synthesis, such as tris(dibenzylideneacetone)dipaddadium (0)/(R)-(+)-2,2′-bis(diphenylphosphino)-1,1′-binaphthyl (Buchwald, S., et al, J. Am. Chem. Soc. 1996, 118, 7215).

Also as shown in Scheme AB-3, compounds of formula A-3-2 and B-3-2 can be made by similar transformations to those described in Scheme 3 from the appropriate starting materials.

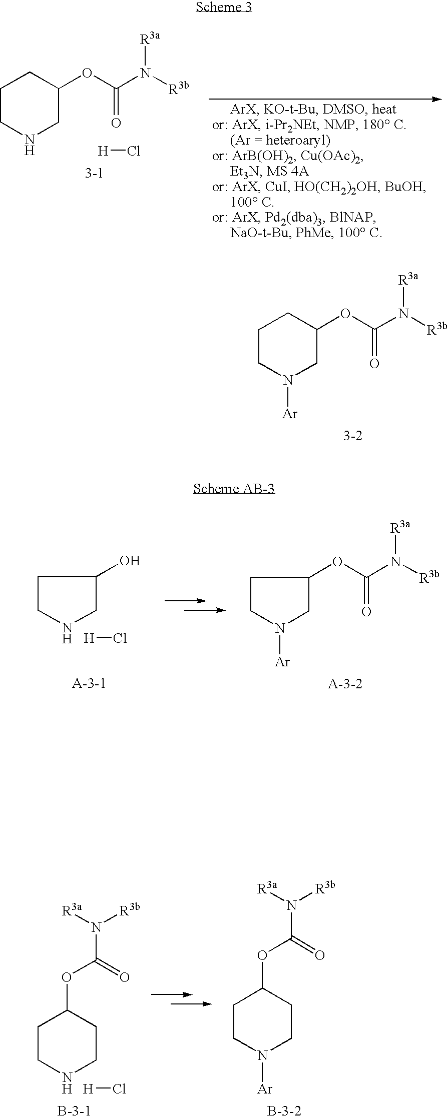

As shown in Scheme 4, alternatively, a series of O-(piperidin-3-yl)carbamates of formula 4-4 (same as 3-2 in Scheme 3) can be prepared in a similar fashion as described in Scheme 3 but with a change of the coupling sequences. Also as shown in Scheme AB-4, compounds of formula A-4-4 and B-4-4 can be made by similar transformations to those described in Scheme 4 starting from the appropriate alcohols.

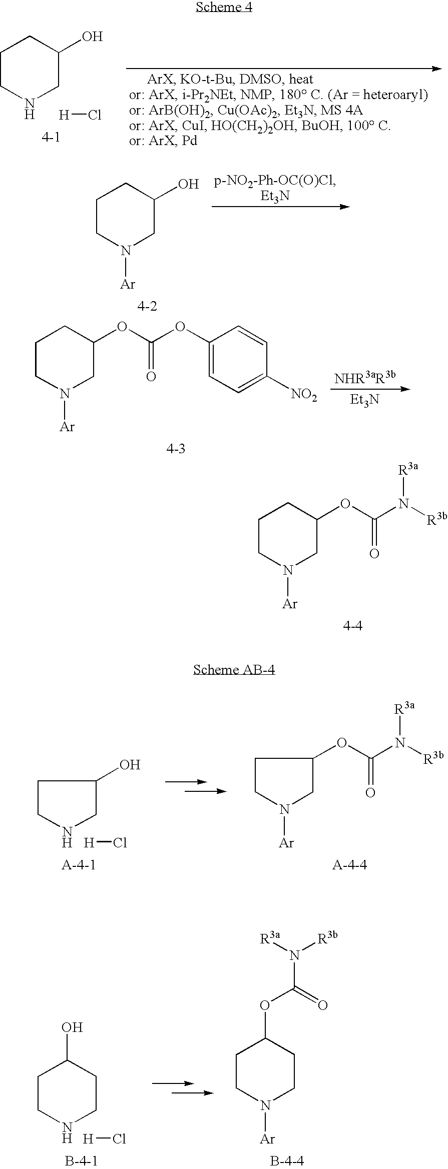

Alternatively, a series of carbamates of formula 5-5 (same as 4-4 in Scheme 4 and 3-2 in Scheme 3) can be prepared according to the method outlined in Scheme 5. Treatment of 2-hydroxy glutaric acid or a salt thereof (such as compound 5-l) with an amine ArNH, (such as aniline or a heteraryl amine) in the presence of a suitable coupling reagent such as EDC provides an imide 5-2, which upon reduction yields a 3-hydroxypiperidine derivative 5-3. Coupling of the 3-hydroxylpiperidine derivative 5-3 to a desired amine NHR^{3a}R^{3b} through an activated p-nitrophenyl carbonic acid ester intermediate 5-4 affords the desired product 5-5.

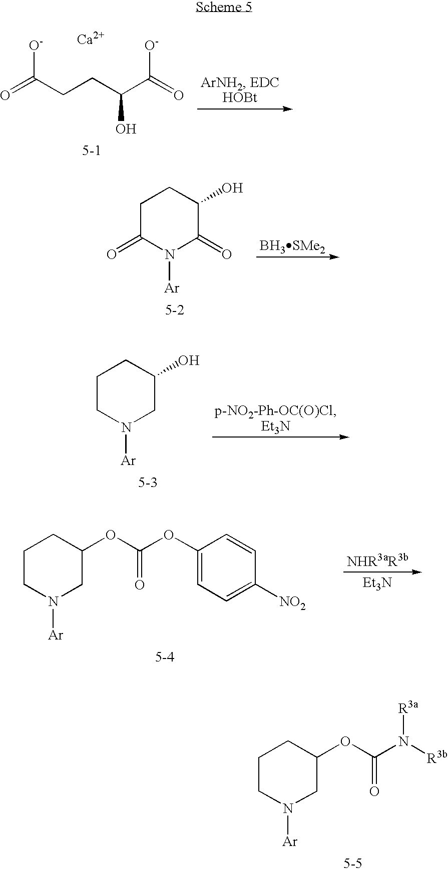

A series of 5-substituted 3-hydroxypiperidines of formula 6-10 can be prepared according to the method outlined in Scheme 6. Reacting 2-hydroxy glutaric acid dimethyl ester 6-1 with benzyl bromide gives the benzyl-protected compound 6-2. Treatment of the compound 6-2 with an alkyl halide RX¹ (wherein R can be alkyl optionally substituted by OH, CN, etc., and X¹ is bromide or iodide) in the presence of suitable base such as sodium hydride, LDA or LiHMDS, and in a suitable solvent such as DMF or THF, provides 4-alkyl dimethyl ester 6-3. Reduction of the ester group of the compound 6-3 with a suitable reducing reagent such as LiAlH₄ affords a bis-hydroxyl compound 6-4. The hydroxyl groups of compound 6-4 can be converted to a better leaving group such as OMs by reacting the compound 6-4 with MsCl under a suitable condition to afford a compound of 6-5. The desired 5-substituted 3-hydroxylpiperidines 6-7 can be prepared by treatment of compound 6-5 with benzylamine followed by palladium catalytic hydrogenation. The 5-substituted 3-hydroxylpiperidine 6-5 can then be transformed to O-(piperidin-3-yl)carbamates of formula 6-10 (wherein L can be a bond (i.e., absent), S(O)₂, S(O), S, S(O)₂NH, C(O), C(O)O, C(O)O—(C₁₋₃ alkylene), C(O)NH, etc.). Alternatively, the bismesylate compound 6-5 can be reacted with ArNH2 (such as aniline or a heteroaryl amine) to provide a compound 6-8, which after removal of the benzyl group can be converted into a compound of formula 6-10 wherein L is absent (i.e., a bond).

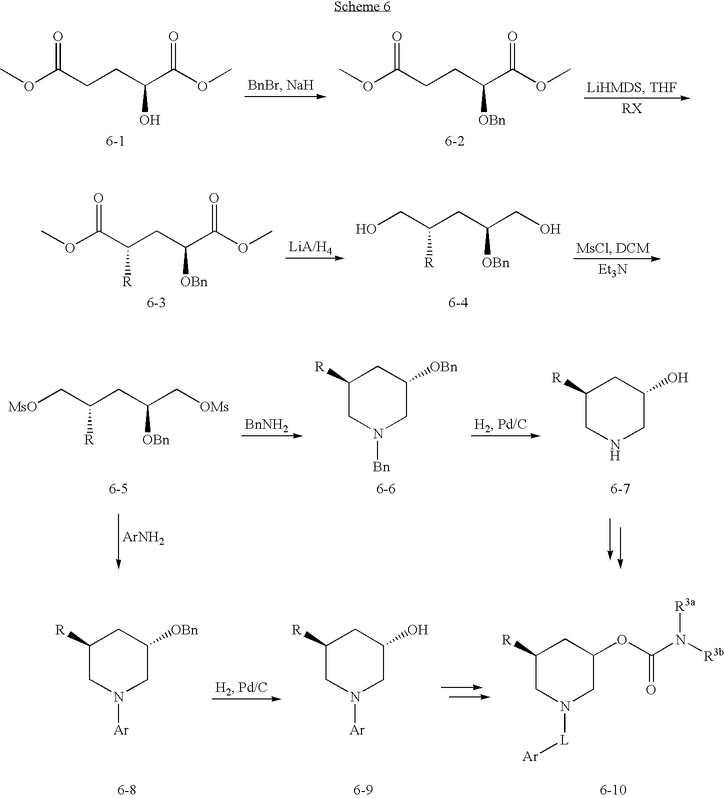

A series of spiro-3-hydroxypiperidines of formula 7-7 can be prepared in a similar manner as shown in Scheme 7 wherein r can be 1, 2, 3, 4 or 5. A diester compound 7-1 can be reacted with a dihalide compound such as a dibromoalkyl compound Br(CH₂)ᵣCH₂Br in a suitable solvent such as THF, and in the presence of a suitable base such as LiHMDS to afford a cycloalkyl compound 7-2. The ester groups of the compound 7-2 can be reduced by a suitable reducing reagent such as LiAlH₄ to afford a di-hydroxyl compound of 7-3. A spiro-compound 7-7 can be obtained from the di-hydroxyl compound 7-3 by using similar procedures to those outlined in Scheme 6.

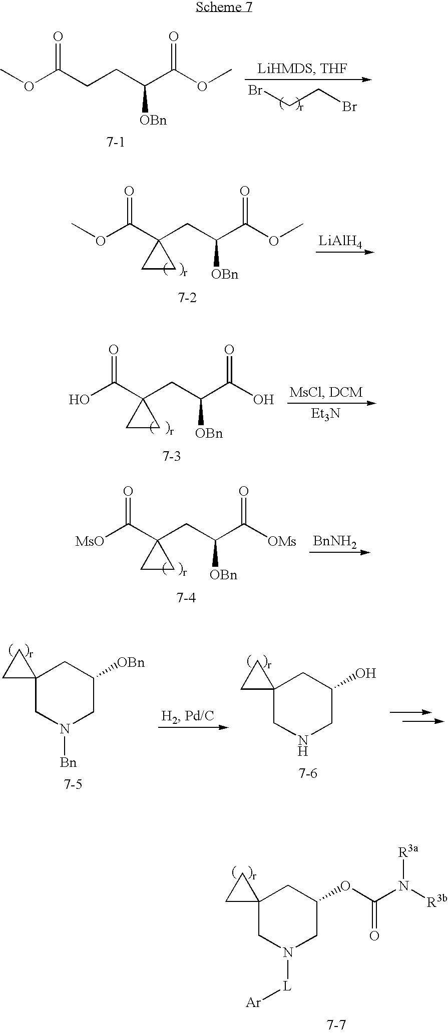

A series of 3-substituted-3-hydroxypiperidines of formula 8-4 can be prepared according to the method outlined in Scheme 8 wherein R¹ can be alkyl, aryl, arylalkyl, cycloalkyl, cycloalkylalky, etc. A ketone compound 8-1 can be treated with a Grignard reagent such as R¹MgBr to afford the compound 8-2. The benzyl group of the compound 8-2 can be removed by hydrogenation with palladium as catalyst to afford the desired 3-substituted 3-hydroxyl-piperidine derivative 8-3. The piperidines 8-4 can further be transformed to O-(piperidin-3-yl)carbamates of formula 8-4 by methods similar to those described hereinabove. Also as shown in Scheme AB-8, compounds of formula A-8-4 and B-8-4 can be made by similar transformations to those described in Scheme 8 from the appropriate starting materials.

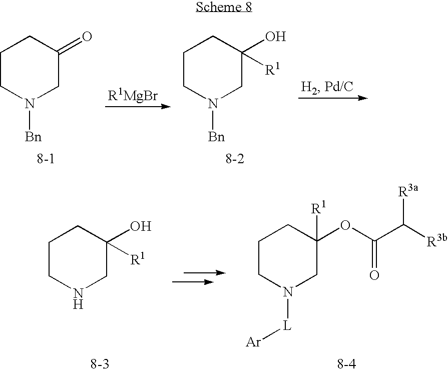

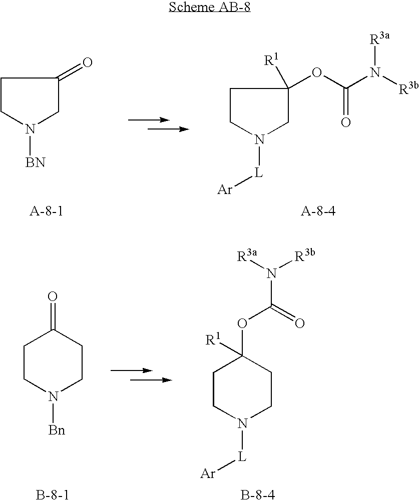

A series of piperidin-3-yl acetamide compounds of formula 9-4 can be prepared according to the method outlined in Scheme 9. (1-Boc-piperidin-3-yl)acetic acid 9-1 can be converted to an amide compound 9-2 in the presence of a suitable coupling reagent for amide-bond formation and in a suitable organic solvent, such as a polar aprotic organic solvent (e.g., N,N-dimethylformamide). Some non-limiting examples of suitable coupling reagents include 1,1′-carbonyl-diimidazole, N-(dimethylaminopropyl)-N′-ethyl carbodiimde, benzotriazol-1-yloxy-tris(dimethylamino)phosphonium hexafluorophosphate (BOP), 1-ethyl-3-(3-dimethylaminopropyl)-carbodiimide (EDC), and propanephosphonic anhydride. Alternatively, acid 9-1 can be treated with thionyl chloride or oxalyl chloride to yield an acid chloride intermediate, which in turn can be reacted with an amine NHR^{3a}R^{3b} in the presence of a suitable base such as triethylamine or pyridine to generate the corresponding amide 9-2. The Boc protecting group of the compound 9-2 can be removed under a suitable condition such as by treatment with HCl in 1,4-dioxane or by treatment with trifluoroacetic acid to afford the corresponding HCl salt 9-3 or the corresponding TFA salt. The HCl salt 9-3 can then be converted to a compound of formula 9-4 using procedures analogous to those described in Scheme 3. 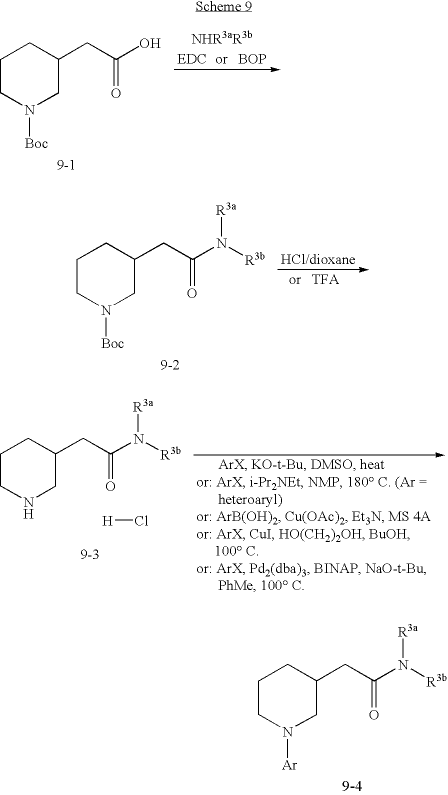
Methods

Compounds of the invention can modulate activity of 11βHSD1. The term “modulate” is meant to refer to an ability to increase or decrease activity of an enzyme. Accordingly, compounds of the invention can be used in methods of modulating 11βHSD1 by contacting the enzyme with any one or more of the compounds or compositions described herein. In some embodiments, compounds of the present invention can act as inhibitors of 11βHSD1. In further embodiments, the compounds of the invention can be used to modulate activity of 11βHSD1 in an individual in need of modulation of the enzyme by administering a modulating amount of a compound of the invention.

The present invention further provides methods of inhibiting the conversion of cortisone to cortisol in a cell, or inhibiting the production of cortisol in a cell, where conversion to or production of cortisol is mediated, at least in part, by 11βHSD1 activity. Methods of measuring conversion rates of cortisone to cortisol and vice versa, as well as methods for measuring levels of cortisone and cortisol in cells, are routine in the art.

The present invention further provides methods of increasing insulin sensitivity of a cell by contacting the cell with a compound of the invention. Methods of measuring insulin sensitivity are routine in the art.

The present invention further provides methods of treating disease associated with activity or expression, including abnormal activity and overexpression, of 11βHSD1 in an individual (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the enzyme or receptor. An 11βHSD1-associated disease can also include any disease, disorder or condition that can be prevented, ameliorated, or cured by modulating enzyme activity.

Examples of 11βHSD1-associated diseases include obesity, diabetes, glucose intolerance, insulin resistance, hyperglycemia, hypertension, hyperlipidemia, cognitive impairment, dementia, depression (e.g., psychotic depression), glaucoma, cardiovascular disorders, osteoporosis, and inflammation. Further examples of 11βHSD1-associated diseases include metabolic syndrome, coronary heart disease, type 2 diabetes, hypercortisolemia, androgen excess (hirsutism, menstrual irregularity, hyperandrogenism) and polycystic ovary syndrome (PCOS). In some embodiments, the disease is obesity. In some embodiments, the disease is diabetes.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is an adipocyte, a pancreatic cell, a hepatocyte, neuron, or cell comprising the eye.

As used herein, the term “contacting” refers to the bringing together of indicated moieties in an in vitro system or an in vivo system. For example, “contacting” the 11βHSD1 enzyme with a compound of the invention includes the administration of a compound of the present invention to an individual or patient, such as a human, having 11βHSD1, as well as, for example, introducing a compound of the invention into a sample containing a cellular or purified preparation containing the 11βHSD1 enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein the term “treating” or “treatment” refers to 1) preventing the disease; for example, preventing a disease, condition or disorder in an individual who may be predisposed to the disease, condition or disorder but does not yet experience or display the pathology or symptomatology of the disease; 2) inhibiting the disease; for example, inhibiting a disease, condition or disorder in an individual who is experiencing or displaying the pathology or symptomatology of the disease, condition or disorder (i.e., arresting further development of the pathology and/or symptomatology), or 3) ameliorating the disease; for example, ameliorating a disease, condition or disorder in an individual who is experiencing or displaying the pathology or symptomatology of the disease, condition or disorder (i.e., reversing the pathology and/or symptomatology).

Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, antibodies, immune suppressants, anti-inflammatory agents and the like.

Labeled Compounds and Assay Methods

Another aspect of the present invention relates to labeled compounds of the invention (radio-labeled, fluorescent-labeled, etc.) that would be useful not only in radio-imaging but also in assays, both in vitro and in vivo, for localizing and quantitating the enzyme in tissue samples, including human, and for identifying ligands by inhibition binding of a labeled compound. Accordingly, the present invention includes enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³, ¹²⁴I , ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from ³H, ¹⁴C, ¹²⁵I, ³⁵S and ⁸²Br.

In some embodiments, the labeled compounds of the present invention contain a fluorescent table.

Synthetic methods for incorporating radio-isotopes and fluorescent labels into organic compounds are are well known in the art.

A labeled compound of the invention (radio-labeled, fluorescent-labeled, etc.) can be used in a screening assay to identify/evaluate compounds. For example, a newly synthesized or identified compound (i.e., test compound) which is labeled can be evaluated for its ability to bind a 11βHSD1 by monitering its concentration variation when contacting with the 11βHSD1, through tracking the labeling. For another example, a test compound (labeled) can be evaluated for its ability to reduce binding of another compound which is known to bind to 11βHSD1 (i.e., standard compound). Accordingly, the ability of a test compound to compete with the standard compound for binding to the 11βHSD1 directly correlates to its binding affinity. Conversely, in some other screening assays, the standard compound is labled and test compounds are unlabeled. Accordingly, the concentration of the labled standard compound is monitored in order to evaluate the competition between the standard compound and the test compound, and the relative binding affinity of the test compound is thus ascertained.

Kits

The present invention also includes pharmaceutical kits useful, for example, in the treatment or prevention of 11βHSD1-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. The compounds of the Examples were found to be inhibitors of 11βHSD1 according to one or more of the assays provided herein.

### EXAMPLES

### Example 1

### 1-(1-naphthylsulfonyl)piperidin-3-yl-piperidine-1-carboxylate

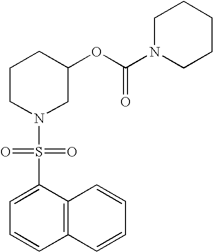

### Step 1. 1-(1-naphthylsulfonyl)piperidin-3ol

To a mixture of (3S)-piperidin-3-ol hydrochloride (0.100 g, 0.000727 mol) in 1.00 M of sodium hydroxide in water (2.18 mL) and methylene chloride (3.00 mL, 0.0468 mol) was added 1-naphthalene sulfonylchloride (0.165 g, 0.000727 mol). The reaction mixture was stirred at rt overnight, and extracted with methylene chloride. The organic layers were combined, washed with brine, dried, and evaporated to dryness. The crude mixture was used directly in next step (203 mg, 95.87%). LCMS (M+H) 292.1.

### Step 2. 1-(1-naphthylsulfonyl)piperidin-3-yl piperidine-1-carboxylate

To a mixture of 1-(1-naphthylsulfonyl)piperidin-3-ol (30.0 mg, 0.000103 mol) in methylene chloride (0.50 mL, 0.0078 mol) was added N,N-carbonyldiimidazole (18.4 mg, 0.000113 mol). The reaction was stirred at rt for 2 h, LCMS (M+H) 386.2. for the imidazole intermediate. The reaction mixture was then treated with piperidine (0.0153 mL, 0.000154 mol) at rt overnight. After evaporation to dryness, the residue was diluted with acetonitrile (AcCN) and water and applied on RP-HPLC to give the desired product (38 mg, 92%). LCMS (M+H) 403.2. The final product was believed to have 3S stereochemistry based on the starting material.

### Example 2

### 1-(1-naphthylsulfonyl)piperidin-3-yl 4-hydroxypiperidine-1-carboxylate

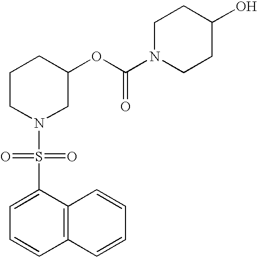

This compound was prepared using procedures analogous to those for examples 1. LCMS (M+H): 419.2.

### Example 3

### 1-(1-naphthylsulfonyl)piperidin-3-yl-3-hydroxy-8-azabicyclo[3.2.1]octane-8-carboxylate

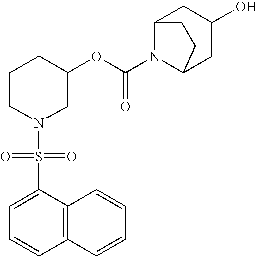

### Step 1. tert-butyl-3-hydroxy-8-azabicyclo[3.2.1]octane-8-carboxylate

tert-Butyl 3-oxo-8-azabicyclo[3.2.1]octane-8-carboxylate (20.0 g, 0.0888 mol) was dissolved in tetrahydrofuran (129.4 mL, 1.596 mol) and the reaction mixture was cooled to −72° C. (internal temperature). To the reaction mixture was added diisobutylaluminum hydride in hexane (1.0 M, 120 mL) dropwisely over 30 min, and the temperature was kept below −63 ° C. The mixture was stirred at a temperature of less than −70° C. for an additional 3.5 hours; and LCMS showed predominantly axial alcohol. The reaction mixture was quenched with water (2.5 mL). The cold bath was removed, and the reaction mixture was warmed to −30° C., and more water (2.5 mL) was added. After the temperature of the mixture reached −20° C., bubbling ceased. An additional 6 mL of water was added slowly and the reaction mixture was warmed to 0° C., transferred to separatory funnel, and diluted with ethylacetate (EtOAc) and water. Then saturated sodium potassium tartrate was added to break up the resulting emulsion/gel. The layers were separated and the aqueous layer was washed with EtOAc. The organic layers were combined, dried (over Na₂SO₄), filtered, and concentrated to give a white solid. The solid was crystallized twice from methylene chloride to give the pure product (15 g, 74.33%) which was believed to have an endo configuration. LCMS (M+Na) 250.2.

### Step 2. 8-azabicyclo[3.2.1]octane-3-ol hydrochloride

tert-Butyl-3-hydroxy-8-azabicyclo[3.2.1]octane-8-carboxylate (15.0 g, 0.0660 mol) was treated with hydrogen chloride in 1,4-dioxane (4.00 M, 82.5 mL) at room temperature (rt) overnight. After evaporation to dryness, the resulting HCl salt was used directly in next step (10.7 g, 99.08%). LCMS (M+H): 128.2.

### Step 3. 1-(1-naphthylsulfonyl)piperidin-3-yl-3-hydroxy-8-azabicyclo[3.2.1]octane-8-carboxylate

This compound was prepared using procedures analogous to those for examples 1. LCMS (M+H): 445.2. The product was believed to have 3S stereochemistry and 3-endo configuration based on the starting material.

### Example 4

### 1-(2-fluoro-4-nitrophenyl)piperidin-3-yl-3-hydroxy-8-azabicyclo[3.2.1]octane-8-carboxylate

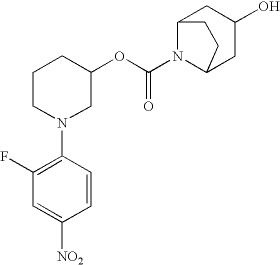

### Step 1. 1-(2-fluoro-4-nitrophenyl)piperidin-3-ol

To a stirred solution of (3S)-piperidin-3-ol hydrochloride (2.000 g, 0.01453 mol) in N,N-dimethylformamide (17.46 mL, 0.2256 mol) were added 1,2-difluoro-4-nitrobenzene (2.43 g, 0.0153 mol) and potassium carbonate (5.02 g, 0.0363 mol). The stirring continued at 90° C. for 13 h. After the reaction mixture was cooled, the mixture was diluted with EtOAc and washed with water and brine. The organic layers were dried and concentrated in vacuo. The resultant residue was used in the next step (3.35 g, 95%). An analytically pure sample was purified on RP-HPLC. LCMS (M+H): 241.2.

### Step 2. 1-(2-fluoro-4-nitrophenyl)piperidin-3-yl-3-hydroxy-8-azabicyclo[3.2.1]octane-8-carboxylate

To a mixture of 1-(2-fluoro-4-nitrophenyl)piperidin-3-ol (300.0 mg, 0.001249 mol) and p-nitrophenyl chloroformate (277 mg, 0.00137 mol) in methylene chloride (5.16 mL, 0.0804 mol) was added triethylamine (0.522 mL, 0.00375 mol). The mixture was stirred at rt for 2 h, then concentrated to dryness. The residue was diluted with 5 mL of dimethylformamide (DMF) and treated with (3-endo)-8-azabicyclo[3.2.1]octan-3-ol hydrochloride (0.245 g, 0.00150 mol) and 0.5 mL of triethylamine (TEA) at rt overnight. The reaction mixture was applied on RP-HPLC to give the desired product (362 mg, 74%). LCMS (M+H): 394.2. The product was believed to have 3S stereochemistry and 3-endo configuration based on the starting materials.

### Example 5

### 1-(4-amino-2-fluorophenyl)piperidin-3-yl-3-hydroxy-8-azabicyclo[3.2.1]octane-8-carboxylate

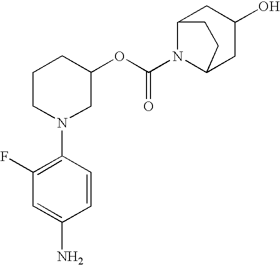

A mixture of 1-(2-fluoro-4-nitrophenyl)piperidin-3-yl-3-hydroxy-8-azabicyclo[3.2.1]octane-8-carboxylate (0.300 g, 0.000762 mol) (see Ex. 4) in 5 mL of MeOH was hydrogenated in the presence of 30 mg of 10% Pd/C, under a balloon of hydrogen overnight. After the catalyst was filtered off, the filtrate was concentrated to dryness and the residue was used directly in next step (0.274 g, 99%). An analytically pure sample was obtained by RP-HPLC. LCMS (M+H): 364.2. The product was believed to have 3S stereochemistry and 3-endo configuration based on the starting material.

### Example 6

### 1-(2-fluoro-4-[(isopropoxycarbonyl)amino]phenyl)piperidin-3-yl-3-hydroxy-8-azabicyclo[3.2.1]octane-8-carboxylate

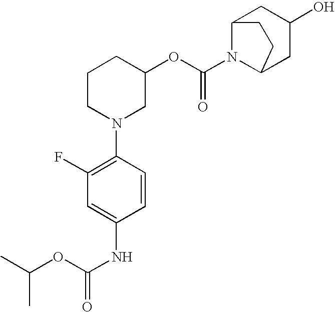

To a mixture of 1-(4-amino-2-fluorophenyl)piperidin-3-yl-3-hydroxy-8-azabicyclo[3.2.1]octane-8-carboxylate (20.0 mg, 0.0000552 mol) in methylene chloride (0.25 mL, 0.0039 mol) was added 1.00 M of sodium hydroxide in water (0.08277 mL), followed by isopropyl chloroformate (0.00845 g, 0.0000690 mol). The reaction mixture was stirred at rt for 1 h, then evaporated to dryness. The residue was purified on RP-HPLC to give the desired product (23 mg, 93%). LCMS (M+H): 450.3. The product was believed to have 3S stereochemistry and 3-endo configuration based on the starting materials.

### Example 7

### 1-(2-fluoro-4-[(methoxycarbonyl)amino]phenyl)piperidin-3-yl-3-hydroxy-8-azabicyclo[3.2.1]octane-8-carboxylate

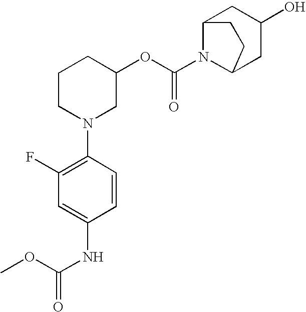

This compound was prepared using procedures analogous to those for Example 6. LCMS (M+H): 422.2.

### Example 8

### 1-(4-[(ethoxycarbonyl)amino]-2-fluorophenyl)piperidin-3-yl-3-hydroxy-8-azabicyclo[3.2.1]octane-8-carboxylate

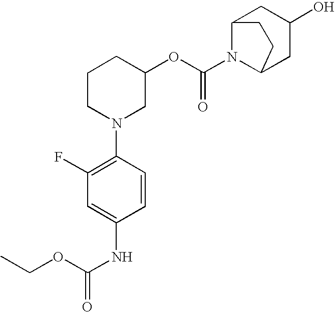

This compound was prepared using procedures analogous to those for Example 6. LCMS (M+H): 436.3.

### Example 9

### 1-(2-fluoro-4-[(propoxycarbonyl)amino]phenyl)piperidin-3-yl-3-hydroxy-8-azabicyclo[3.2.1]octane-8-carboxylate

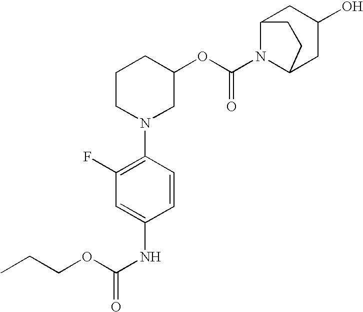

This compound was prepared using procedures analogous to those for Example 6. LCMS (M+H): 450.3.

### Example 10

### 1-(2-fluoro-4-[(isobutoxycarbonyl)amino]phenyl)piperidin-3-yl-3-hydroxy-8-azabicyclo[3.2.1]octane-8-carboxylate

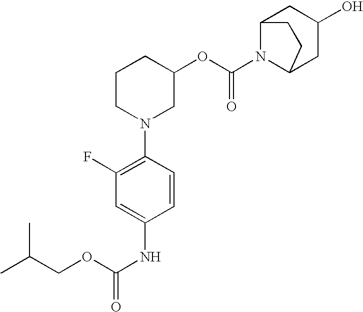

This compound was prepared using procedures analogous to those for Examples 6. LCMS (M+H): 464.3.

### Example 11

### 1-[2-fluoro-4-(2-oxopyrrolidin-1-yl)phenyl]piperidin-3-yl-3-hydroxy-8-azabicyclo[13.2.1]octane-8-carboxylate

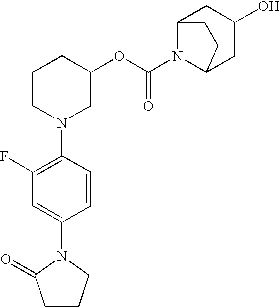

To a mixture of 1-(4-amino-2-fluorophenyl)piperidin-3-yl-3-hydroxy-8-azabicyclo[3.2.1]octane-8-carboxylate (20.0 mg, 0.0000552 mol) and 4-dimethylaminopyridine (10.11 mg, 8.277E-5 mol) in tetrahydrofuran (0.51 mL, 0.0062 mol) was added 4-bromo-butanoyl chloride, (0.00798 mL, 0.0000690 mol). The mixture was stirred at rt for I h, then treated with 1.00 M of potassium tert-butoxide in tetrahydrofuran (THF) (0.221 mL) at rt for 2 h, then evaporated to dryness. The residue was purified on RP-HPLC to give the product (20 mg, 83%). LCMS (M+H): 432.2. The product was believed to have 3S stereochemistry and 3-endo configuration based on the starting materials.

### Example 12

### 1-[2-fluoro-4-(2-oxo-1,3-oxazolidin-3-yl)phenyl]piperidin-3-yl-3-hydroxy-8-azabicyclo[3.2.1]octane-8-carboxylate

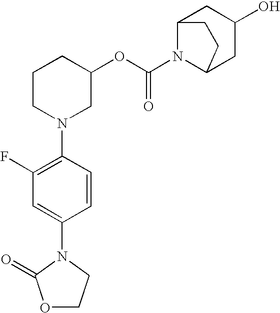

This compound was prepared using procedures analogous to those for examples 11. LCMS (M+H): 434.2.

### Example 13

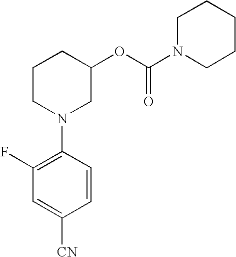

### 1-(4-cyano-2-fluorophenyl)piperidin-3-yl piperidine-1-carboxylate

### Step 1. 3-fluoro-4-[3-hydroxypiperidin-1-yl]benzonitrile

A mixture of (3S)-piperidin-3-ol hydrochloride (60.0 mg, 0.000436 mol), 3,4-difluorobenzonitrile (66.7 mg, 0.000480 mol) and potassium carbonate (151 mg, 0.00109 mol) in N,N-dimethylformamide (2.1 mL, 0.027 mol) was heated at 120° C. overnight. After quenching with water, the mixture was extracted with EtOAc. The organic layers were combined, washed with water, brine, dried, and evaporated to dryness. The crude residue was used directly in next step (88 mg. 92%). LCMS (M+H): 221.2.

### Step 2. 1-(4-cyano-2-fluorophenyl)piperidin-3-yl piperidine-1-carboxylate

To a mixture of 3-fluoro-4-[3-hydroxypiperidin-1-yl]benzonitrile (30.0 mg, 0.000136 mol) and p-nitrophenyl chloroformate (30.2 mg, 0.000150 mol) in methylene chloride (0.562 mL, 0.00878 mol) was added triethylamine (0.0570 mL, 0.000409 mol). The mixture was stirred at rt for 1 h (LCMS (M+H) 386.1 indicated the formation of the carbonate intermediate).

To the resulting mixture was added piperidine (0.0202 mL, 0.000204 mol). The reaction was stirred at rt for 2 h, then evaporated to dryness. The residue was diluted with water and AcCN and then purified on RP-HPLC to give the desired product (28 mg, 63%). LCMS (M+H): 332.2. The product was believed to have 3S stereochemistry based on the starting material.

### Example 14

### 1-(4-cyano-2-fluorophenyl)piperidin-3-yl-4-hydroxypiperidine-1-carboxylate

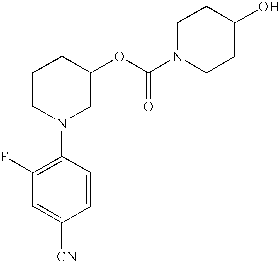

This compound was prepared using procedures analogous to those for Example 13. LCMS (M+H)L 348.2.

### Example 15

### 1-(4-cyano-2-fluorophenyl)piperidin-3-yl-3-hydroxy-8-azabicyclo[3.2.1]octane-8-carboxylate

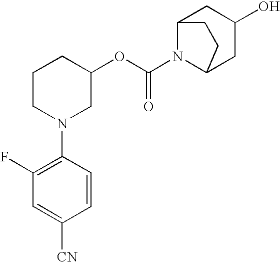

This compound was prepared using procedures analogous to those for Example 13. LCMS (M+H): 374.2.

### Example 16

### 1-(4-[(cyclohexylcarbonyl)amino]-2-fluorophenyl)piperidin-3-yl-3-hydroxy-8-azabicyclo[3.2.1]octane-8-carboxylate

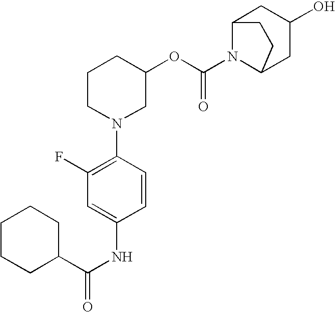

To a mixture of 1-(4-amino-2-fluorophenyl)piperidin-3-yl-3-hydroxy-8-azabicyclo[3.2.1]octane-8-carboxylate (20.0 mg, 0.0000550 mol) in methylene chloride (0.50 mL, 0.0078 mol) was added 4-dimethylaminopyridine (10.08 mg, 8.255E-5 mol), followed by cyclohexanecarbonyl chloride (9.35 μL, 0.0000688 mol). The reaction was stirred at rt for 1 h then evaporated to dryness. The residue was diluted with MeOH and treated with 1 N LiOH at rt overnight 3 days. The resulting mixture was purified on RP-HPLC to give the desired product (18 mg, 69%). LCMS (M+H): 474.3.

### Example 17

### 1-(4-1(cyclopentylcarbonyl)amino]-2-fluorophenyl)piperidin-3-yl-3-hydroxy-8-azabicyclo[3.2.1]octane-8-carboxylate

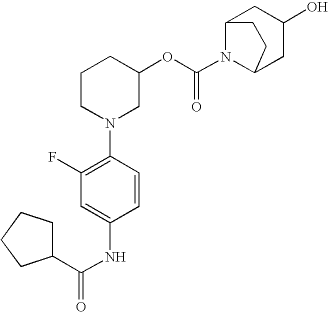

This compound was prepared using procedures analogous to those for Example 16. LCMS (M+H): 460.3.

### Example 18

### 1-(4-[(cyclobutylcarbonyl)amino]-2-fluorophenyl)piperidin-3-yl-3-hydroxy-8-azabicyclo[3.2.1]octane-8-carboxylate

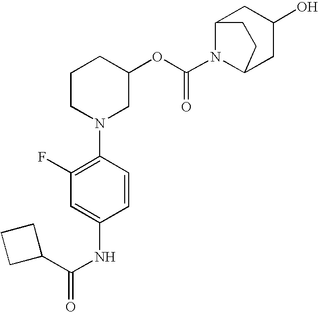

This compound was prepared using procedures analogous to those for Example 16. LCMS (M+H): 446.3.

### Example 19

### 1-(4-[(cyclopropylcarbonyl)amino]-2-fluorophenyl)piperidin-3-yl-3-hydroxy-8-azabicyclo[3.2.1]octane-8-carboxylate

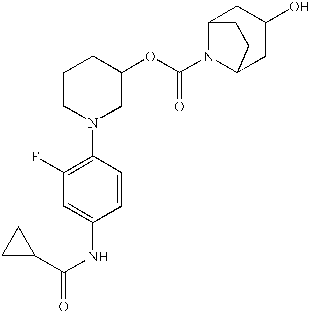

This compound was prepared using procedures analogous to those for Example 16. LCMS (M+H): 432.3.

### Example 20

### 1-[4-(cyclopentanecarbonyl-amino)-2-fluoro-phenyl]-piperidin-3-yl-piperidine-1-carboxylate

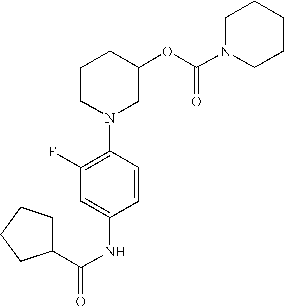

This compound was prepared using procedures analogous to those for Example 16. LCMS (M+H): 417.3.

### Example 21

### 1-(4-cyano-2,6difluorophenyl)piperidin-3-yl-piperidine-1-carboxylate

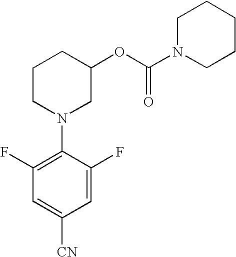

### Step 1. 3,5-difluoro-4-[3-hydroxypiperidin-1-yl]benzonitrile

A mixture of (3S)-piperidin-3-ol hydrochloride (68.5 mg, 0.000498 mol), 3,4,5-trifluorobenzonitrile (86.0 mg, 0.000547 mol) and potassium carbonate (172 mg, 0.00124 mol) in N,N-dimethylformamide (2.4 mL, 0.031 mol) was heated at 120° C. overnight. After quenching with water, the mixture was extracted with EtOAc. The organic layers were combined, washed with water, brine, dried, and evaporated to dry. The crude residue was used directly in next step (110 mg, 93%). LCMS (M+H): 239.2.

### Step 2. 1-(4-cyano-2,6-difluorophenyl)piperidin-3-yl piperidine-1-carboxylate

To a mixture of 3,5-difluoro-4-[3-hydroxypiperidin-1-yl]benzonitrile (32.4 mg, 0.000136 mol) and p-nitrophenyl chloroformate (30.2 mg, 0.000150 mol) in methylene chloride (0.562 mL, 0.00878 mol) was added triethylamine (0.0570 mL, 0.000409 mol). The mixture was stirred at rt for 1 h. To the resulting mixture was added piperidine (0.0202 mL, 0.000204 mol). The reaction was stirred at rt for 2 h, then evaporated to dryness. The residue was diluted with water and AcCN and purified on RP-HPLC to give the desired product (32 mg, 67%). LCMS (M+H): 350.2. The product was believed to have 3S stereochemistry based on the starting material.

### Example 22

### 1-(4-cyano-2,6-difluorophenyl)piperidin-3-yl-4-hydroxypiperidine-1-carboxylate

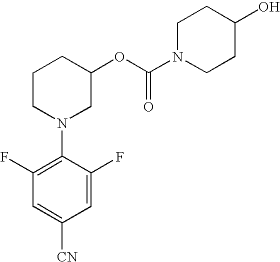

This compound was prepared using procedures analogous to those for Example 21. LCMS (M+H): 366.2.

### Example 23

### 1-(4-cyano-2,6-difluorophenyl)piperidin-3-yl-3-hydroxy-8-azabicyclo[3.2.1] octane-8-carboxylate

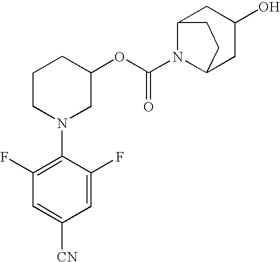

This compound was prepared using procedures analogous to those for Example 21. LCMS (M+H): 392.2.

### Example 24

### 1-(4-cyano-2-fluorophenyl)piperidin-3-yl-3-hydroxy-9-azabicyclo[3.3.1]nonane-9-carboxylate

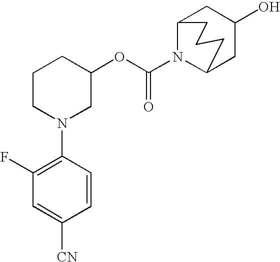

### Step 1. 9-benzyl-9-azabicyclo[3.3.1]nonan-3-one

1,3-Acetonedicarboxylic acid (50.0 g, 0.342 mol) was added to a solution of glutaric dihydride (68.6 g, 0.342 mol) in water (50%) and benzylamine hydrochloride (58.9 g, 0.410 mol) in water (146 mL, 8.11 mol) at 0° C., after which a solution of sodium acetate (11 g, 0.14 mol) dissolved in water (114 mL, 6.31 mol) (10% of sodium acetate) was added to the reaction mixture. The mixture was stirred for 1 h at rt and then for 4 h at 50° C. After this the reaction mixture was adjusted to pH 2 with 10% HCl and then washed with ether (3×200 mL); it was then adjusted to pH 6 with sodium bicarbonate and extracted with methylene chloride (3×200 mL). The combined organic extracts were dried and evaporated to give a pale orange paste, which was taken up in hot ether (10×150 mL). The ether solution was concentrated to half volume and the desired product crushed out as pale yellow solid (62.3 g, 79.31%). LCMS (M+H): 230.2. l

### Step 2. 9-benzyl-9-azabicyclo[3.3.1]nonan-3-ol

To a suspension of lithium tetrahydroaluminate (98.5 mg, 0.00260 mol) in dry ether (18.0 mL, 0.171 mol) was added a solution of 9-benzyl-9-azabicyclo[3.3.1]nonan-3-one (0.248 g, 0.00108 mol) in ether dropwise, and the mixture was then heated at reflux with stirring for 2 h. After this the reaction mixture was cooled and the excess reagent was decomposed by the addition of 0.1 mL of water, 0.1 mL of 15% NaOH and 0.3 mL of water, successively. The mixture was stirred at rt overnight, filtered, dried and evaporated to dryness (219 mg, 87.54%). LCMS (M+H): 232.2.

### Step 3. 9-azabicyclo[3.3.1]nonan-3-ol acetate (salt)

A mixture of 9-benzyl-9-azabicyclo[3.3.1]nonan-3-ol (0.220 g, 0.000951 mol) in acetic acid (5.00 mL, 0.0879 mol) was hydrogenated in the presence of 10% Pd/C, under balloon pressure of hydrogen, overnight. After the catalyst was filtered off, the filtrate was concentrated to dryness and the residue was used directly in next step (190 mg, 99.27%). LCMS (M+H): 142.2.

### Step 4. 1-(4-cyano-2-fluorophenyl)piperidin-3-yl-3-hydroxy-9-azabicyclo[3.3.1]nonane-9-carboxylate

To a mixture of 9-azabicyclo[3.3.1]nonan-3-ol acetate (HCl salt) (15.7 mg, 0.0000778 mol) and triethylamine (0.0326 mL, 0.000234 mol) was added 1-(4-cyano-2-fluorophenyl)piperidin-3-yl 4-nitrophenyl nitrophenyl carbonate (30.0 mg, 0.0000778 mol) in methylene chloride (0.60 mL, 0.0094 mol). The reaction mixture was stirred at rt overnight, then concentrated to dryness. The residue was diluted with water and AcCN and purified on RP-HPLC (26 mg, 87%). LCMS: (M+H) 388.2. The product was believed to have 3S stereochemistry and 3-endo configuration based on the starting materials.

### Example 25

### 1-(2,4-difluorophenyl)piperidin-3-yl-piperidine-1-carboxylate

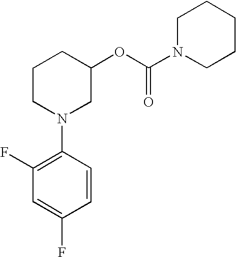

### Step 1. 1-(2,4-difluorophenyl)piperidin-3-ol

A mixture of (3S)-piperidin-3-ol hydrochloride (0.50 g, 0.0036 mol), 1,3-difluoro-4-iodobenzene (0.522 mL, 0.00436 mol), copper(I) iodide (140 mg, 0.00073 mol), potassium phosphate (3.08 g, 0.0145 mol), and 1,2-ethanediol (0.810 mL, 0.0145 mol) in 1-butanol (7.28 mL, 0.0796 mol) was heated at 100° C. under nitrogen for 2 nights. The reaction mixture was treated with water, and then extracted with EtOAc. The organic layers were combined, washed with brine, dried and evaporated to dryness. The residue was used directly in next step without further purifications (529 mg, 69%). LCMS (M+H): 214.2.

### Step 2. 1-(2,4-difluorophenyl)piperidin-3-yl piperidine-1-carboxylate

To a mixture of 1-(2,4-difluorophenyl)piperidin-3-ol (40.0 mg, 0.000188 mol) in methylene chloride (0.800 mL, 0.0125 mol) was added p-nitrophenyl chloroformate (45.4 mg, 0.000225 mol), followed by triethylamine (0.0784 mL, 0.000563 mol). The reaction was stirred at rt for 2 h, and LCMS showed 379.2 (M+H, for the p-nitrophenyl carbonate). The reaction was then treated with piperidine (0.0278 mL, 0.000281 mol) at rt overnight. After evaporating to dryness, the residue was diluted with AcCN and water and purified on RP-HPLC to give the desired product (52 mg, 85%). LCMS (M+H): 325.2. The product was believed to have 3S stereochemistry based on the starting material.

### Example 26

### 1-(2,4-difluorophenyl)piperidin-3-yl-4-hydroxypiperidine-1-carboxylate

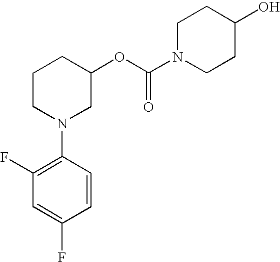

This compound was prepared using procedures analogous to those for Example 25. LCMS (M+H): 341.2.

### Example 27

### 1-(2,4-difluorophenyl)piperidin-3-yl-3-hydroxy-8-azabicyclo[3.2.1]octane-8-carboxylate

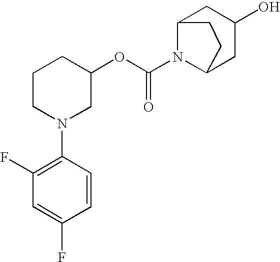

This compound was prepared using procedures analogous to those for Example 25. LCMS (M+H): 367.2.

### Example 28

### ‘-(2,4-difluorophenyl)piperidin-3-yl-3-hydroxy-9-azabicyclo[3.3.1]nonane-9-carboxylate

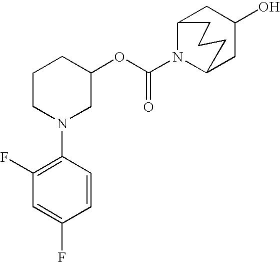

This compound was prepared using procedures analogous to those for Example 25. LCMS (M+H): 381.2.

### Example 29

### 1-(2-fluoro-4-methylphenyl)piperidin-3-yl-piperidine-1-carboxylate

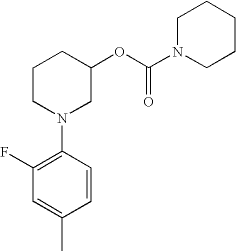

### Step 1. 1-(2-fluoro-4-methylphenyl)piperidin-3-ol

A mixture of (3S)-piperidin-3-ol hydrochloride (0.50 g, 0.0036 mol), 2-fluoro-1-iodo-4-methylbenzene (1.03 g, 0.00436 mol), copper(I) iodide (140 mg, 0.00073 mol), potassium phosphate (3.08 g, 0.0145 mol), and 1,2-ethanediol (0.810 mL, 0.0145 mol) in 1-butanol (7.28 mL, 0.0796 mol) was heated at 100° C. under nitrogen for 2 nights. The reaction mixture was treated with water, and then extracted with EtOAc. The organic layers were combined, washed with brine, dried and evaporated to dryness. The residue was used directly in next step (519 mg, 69%). LCMS (M+H): 210.2.

### Step 2. 1-(2-fluoro-4-methylphenyl)piperidin-3-yl piperidine-1-carboxylate

To a mixture of 1-(2-fluoro-4-methylphenyl)piperidin-3-ol (40.0 mg, 0.000191 mol) in methylene chloride (0.815 mL, 0.0127 mol) was added p-nitrophenyl chloroformate (46.2 mg, 0.000229 mol), followed by triethylamine (0.0799 mL, 0.000573 mol). The reaction mixture was stirred at rt for 2 h, and LCMS shown 375.2 (M+H, for the corresponding p-nitrophenyl carbonate). The reaction mixture was then treated with piperidine (0.0284 mL, 0.000287 mol) at rt overnight. After evaporated to dryness, the residue was diluted with AcCN and water and purified on RP-HPLC to give the desired product (51 mg 84%). LCMS (M+H): 321.2. The product was believed to have 3S stereochemistry based on the starting material.

### Example 30

### 1-(2-fluoro-4-methylphenyl)piperidin-3-yl 4-hydroxypiperidine-1-carboxylate

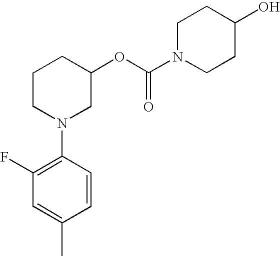

This compound was prepared using procedures analogous to those for Example 29. LCMS (M+H): 337.2.

### Example 31

### 1-(2-fluoro-4-methylphenyl)piperidin-3-yl-3-hydroxy-8-azabicyclo[3.2.1]octane-8-carboxylate

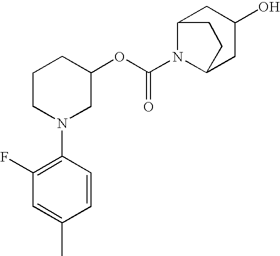

This compound was prepared using procedures analogous to those for Example 29 LCMS (M+H): 363.2.

### Example 32

### 1-(2-fluoro-4-methylphenyl)piperidin-3-yl-3-hydroxy-9-azabicyclo[3.3.1]nonane-9-carboxylate

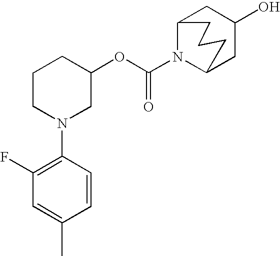

This compound was prepared using procedures analogous to those for Example 29. LCMS (M+H): 377.2.

### Example 33

### 1-(3-methyl-5-nitropyridin-2-yl)piperidin-3-yl-3-hydroxy-8-azabicyclo[3.2.1]octane-8carboxylate

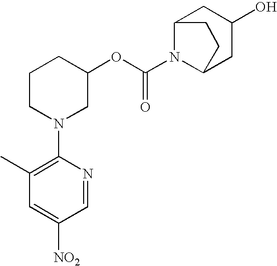

### Step 1. Piperidin-3-yl-3-hydroxy-8-azabicyclo[3.2.1]octane-8-carboxylate hydrochloride

To a mixture of tert-butyl (3S)-3-hydroxypiperidine-l-carboxylate (2.00 g, 0.00994 mol) in methylene chloride (40.0 mL, 0.624 mol) was added p-nitrophenyl chloroformate (2.10 g, 0.0104 mol), followed by triethylamine (4.16 mL, 0.0298 mol). The reaction mixture was stirred at rt for 2 h, and LCMS shown 389.2 (M+Na, for the corresponding p-nitrophenyl carbonate). The reaction mixture was then treated with (3-endo)-8-azabicyclo[3.2.1]octan-3-ol hydrochloride (1.79 g, 0.0109 mol) at rt overnight. After evaporation to dryness, the residue was diluted with EtOAc, washed with 1 N NaOH, water and brine. The organic extract was dried and concentrated to dryness. LCMS (M+Na) 377.2. The crude carbamate was treated with 4.00 M of hydrogen chloride in 1,4-dioxane (12.4 mL) at rt overnight. After evaporated to dryness, the resulting HCl salt was used directly in next step (2.40 g, 82%). LCMS (M+H): 255.2.

### Step 2. 1-(3-methyl-5-nitropyridin-2-yl)piperidin-3-yl-3-hydroxy-8-azabicyclo[3.2.1]octane-8-carboxylate

A mixture of piperidin-3-yl-3-hydroxy-8-azabicyclo[3.2.1]octane-8-carboxylate hydrochloride (0.500 g, 0.00172 mol), 2-chloro-3-methyl-5-nitropyridine (0.312 g, 0.00180 mol), and potassium carbonate (0.356 g, 0.00258 mol) in N,N-dimethylformamide (3.00 mL, 0.0387 mol) was heated at 90° C. overnight. After cooled to rt, the mixture was diluted with EtOAc, washed with water, brine and dried. The resulting residue was used directly in next step. An analytically pure sample was obtained by RP-HPLC (590 mg 88%). LCMS (M+H): 391.2. The product was believed to have 3S stereochemistry and 3-endo configuration based on the starting materials.

### Example 34

### 1-(5-amino-3-methylpyridin-2-yl)piperidin-3-yl-3-hydroxy-8-azabicyclo[3.2.1]octane-8-carboxylate

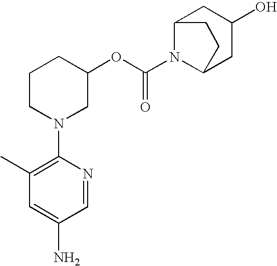

The crude 1-(3-methyl-5-nitropyridin-2-yl)piperidin-3-yl-3-hydroxy-8-azabicyclo[3.2.1]octane-8-carboxylate (0.600 g, 0.00154 mol) in 10 mL of MeOH was hydrogenated in the presence of 10% Pd/C, under a hydrogen balloon for 2 h. After the catalyst was filtered off, the filtrate was concentrated to dryness and used directly in next step. An analytically pure sample was obtained by RP-HPLC (549 mg, 100%). LCMS (M+H): 361.3.

### Example 35

### 1-(5-1(methoxycarbonyl)amino]-3-methylpyridin-2-yl)piperidin-3-yl-3-hydroxy-8-azabicyclo[3.2.1]octane-8-carboxylate

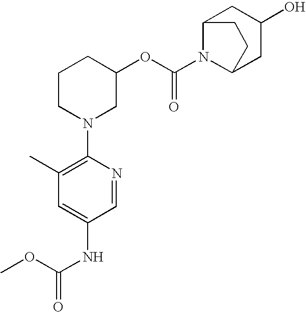

To a mixture of 1-(5-amino-3-methylpyridin-2-yl)piperidin-3-yl-3-hydroxy-8-azabicyclo[3.2.1]octane-8-carboxylate (25.0 mg, 0.0000694 mol) and 1.00 M of sodium hydroxide in water (0.139 mL) in methylene chloride (0.50 mL, 0.0078 mol) was added methyl chloroformate (8.04 μL, 0.000104 mol). The mixture was stirred at rt for 30 min, then the methylene chloride was stripped off. The residue was diluted with AcCN and purified on RP-HPLC to give the desired product (25 mg, 86%). LCMS (M+H): 419.2. The product was believed to have 3S stereochemistry and 3-endo configuration based on the starting material.

### Example 36

### 1-(5-1(ethoxycarbonyl)amino]-3-methylpyridin-2-yl)piperidin-3-yl-3-hydroxy-8-azabicyclo[3.2.1]octane-8-carboxylate

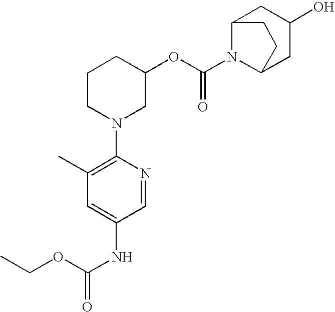

This compound was prepared using procedures analogous to those for Example 35. LCMS (M+H): 433.2.

### Example 37

### 1-(3-methyl-5-[(propoxycarbonyl)amino]pyridin-2-yl)piperidin-3-yl-3-hydroxy-8-azabicyclo[3.2.1]octane-8-carboxylate

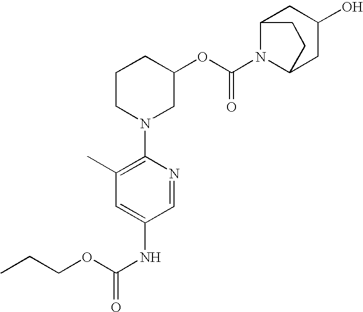

This compound was prepared using procedures analogous to those for Example 35. LCMS (M+H): 447.3.

### Example 38

### 1-(5-[(isopropoxycarbonyl)amino]-3-methylpyridin-2-yl)piperidin-3-yl-3-hydroxy-8-azabicyclo[3.2.1]octane-8-carboxylate

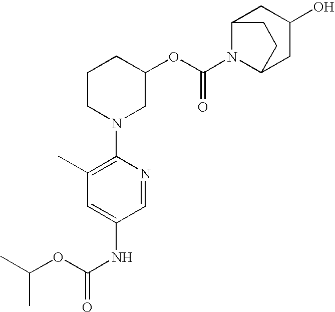

This compound was prepared using procedures analogous to those for Example 35. LCMS (M+H): 447.3.

### Example 39

### 1-(5-[(isobutoxycarbonyl)amino]-3-methylpyridin-2-yl)piperidin-3-yl-3-hydroxy-8-azabicyclo[3.2.1]octane-8-carboxylate

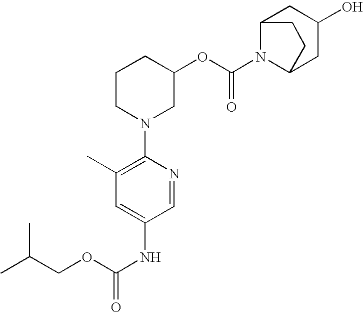

This compound was prepared using procedures analogous to those for Example 35. LCMS (M+H): 461.3.

### Example 40

### 1-(4-cyano-2-fluorophenyl)piperidin-3-yl-2-oxa-6azatricyclo[3.3.1.1(3,7)]decane-6carboxylate

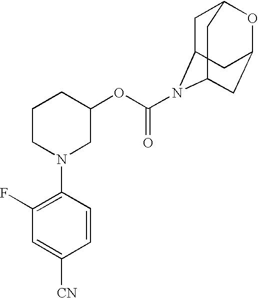

### Step 1. tert-butyl 3-hydroxy-9-azabicyclo[3.3.1]nonane-9-carboxylate

To a mixture of (3-endo)-9-azabicyclo[3.3.1]nonan-3-ol acetate (salt) (10.00 g, 0.04969 mol) and 1.00 M of sodium hydroxide in water (149 mL) in tetrahydrofuran (150.0 mL, 1.849 mol) was added di-tert-butyldicarbonate (16.3 g, 0.0745 mol). The reaction was stirred at rt overnight, then THF was stripped off. The residue was extracted with EtOAc. The organic layers were combined, washed with water, brine, dried, and evaporated to dryness. The residue was chromatogrphed on silica gel, eluting with 0 to 80% EtOAc, to give the desired product (11.3 g, 94.32%). LCMS (M+Na) 264.2.

### Step 2. tert-butyl 2-oxa-6-azatricyclo[3.3.1.1(3,7)]decane-6-carboxylate

A mixture of dry benzene (500.0 mL, 5.594 mol), lead tetraacetate (50.00 g, 0.1128 mol), and calcium carbonate (25.00 g, 0.2498 mol) was heated for 15 min at reflux. A solution of tert-butyl 3-hydroxy-9-azabicyclo[3.3.1]nonane-9-carboxylate (10.60 g, 0.04392 mol) dissolved in benzene (400.00 mL, 4.4756 mol) and iodine (21.00 g, 0.08274 mol) were then added and the refluxing was continued for 3 h. The cooled solution was filtered and the filtrate washed with 10% aq. Na₂S₂O₃ and water. After the solution was dried and evaporated to dryness, the residue was chromatographied on a silica gel column, eluting with 0 to 30% EtOAc in hexane, to give the desired 2-aza-6-oxaadmantane compound (3.69 g, 35%), LCMS (M+Na) 262.2.

### Step 3. 2-oxa-6-azatricyclo[3.3.1.1(3,7)]decane hydrochloride

tert-Butyl 2-oxa-6-azatricyclo[3.3.1.1(3,7)]decane-6-carboxylate (1.90 g, 0.00794 mol) was treated with 4.00 M of hydrogen chloride in 1,4-dioxane (39.7 mL) at rt overnight. After the mixture was evaporated to dryness, the resultant HCl salt (1.39 g, 99.67%) was used directly in next step. LCMS (M+H) 140.0.

### Step 4. 1-(4-cyano-2-fluorophenyl)piperidin-3-yl 2-oxa-6-azatricyclo[3.3.1.1(3,7)]decane-6-carboxylate

To a mixture of the crude 1-(4-cyano-2-fluorophenyl)piperidin-3-yl-4-nitrophenyl carbonate (30.0 mg, 0.0000778 mol), and triethylamine (0.0326 mL, 0.000234 mol) in methylene chloride (1.18 mL, 0.0183 mol) was added 2-oxa-6-azatricyclo[3.3.1.1(3,7)]decane hydrochloride (0.0164 g, 0.0000934 mol). The reaction mixture was stirred at rt overnight. After the mixture was evaporated to dryness, the residue was diluted with AcCN and water, and purified on RP-HPLC to give the desired product (14 mg, 46.7%). LCMS (M+H) 386.0. The product was believed to have 3S stereochemistry based on the starting materials.

### Example 41

### 1-(2-fluoro-4-nitrophenyl)piperidin-3-yl-2-oxa-6-azatricyclo[13.3.1.1(3,7)]decane-6-carboxylate

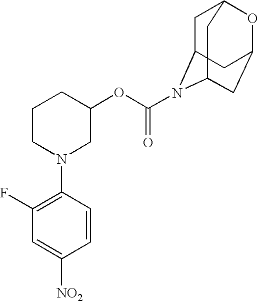

To a mixture of 1-(2-fluoro-4-nitrophenyl)piperidin-3-ol (200.0 mg, 0.0008325 mol) and p-nitrophenyl chloroformate (0.184 g, 0.000916 mol) in methylene chloride (4.00 mL, 0.0624 mol) was added triethylamine (0.464 mL, 0.00333 mol). After the mixture was stirred at rt for 2 h, LCMS showed the formation of the carbamate intermediate, (M+H) 406.1. To the reaction mixture was added 2-oxa-6-azatricyclo[3.3.1.1(3,7)]decane hydrochloride (0.175 g, 0.000999 mol). The resultant mixture was stirred at rt overnight. The mixture was diluted with methylene chloride, washed with 1 N NaOH, brine and dried, evaporated to dryness. The crude residue was used directly in next step (304 mg, 90.07%). An analytically pure sample was obtained by RP-HPLC. LCMS (M+H) 406.2. l

### Example 42

### 1-(2-fluoro-4-methylphenyl)piperidin-3-yl 2-oxa-6-azatricyclo[3.3.1.1(3,7)1decane-6

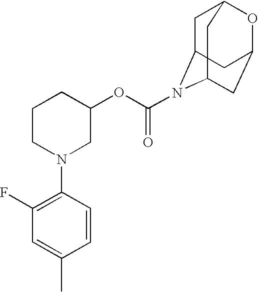

To a mixture of 1-(2-fluoro-4-methylphenyl)piperidin-3-ol (25.0 mg, 0.000119 mol) (see Example 4) and p-nitrophenyl chloroformate (0.0265 g, 0.000131 mol) in methylene chloride (1.00 mL, 0.0156 mol) was added triethylamine (0.0666 mL, 0.000478 mol). After the mixture was stirred at rt for 2 h, LCMS showed the formation of the activated carbonate intermediate, (M+H) 375.1. To the reaction mixture was added 2-oxa-6-azatricyclo[3.3.1.1(3,7)]decane hydrochloride (0.0252 g, 0.000143 mol). The resultant mixture was stirred at rt overnight, and then evaporated to dryness. The residue was purified on RP-HPLC to give the desired product (36 mg, 80.9%). LCMS (M+H) 375.1. The product was believed to have 3S stereochemistry based on the starting material.

### Example 43

### 1-(2,4-difluorophenyl)piperidin-3-yl 2-oxa-6-azatricyclo[3.3.1.1(3,7)]decane-6-carboxylate

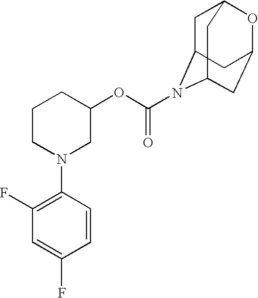

This compound was prepared using procedures analogous to those for examples 25. LCMS (M+H): 379.0.

### Example 44

### 1-(4-amino-2-fluorophenyl)piperidin-3-yl 2-oxa-6-azatricyclo[3.3.1.1(3,7)]decane-6-carboxylate

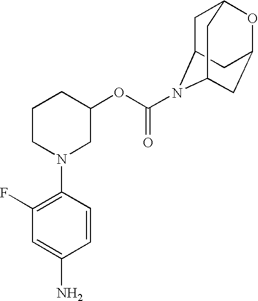

1-(2-Fluoro-4-nitrophenyl)piperidin-3-yl-2-oxa-6-azatricyclo[3.3.1.1(3,7)]decane-6-carboxylate (0.236 g, 0.000582 mol) was hydrogenated in the presence of 10% Pd/C under a hydrogen balloon for 2 h. After the catalyst was filtered off, the filtrate was concentrated to dryness and the residue was used directly in next step (217 mg, 99.29%). An analytically pure sample was obtained by RP-HPLC. LCMS (M+H) 376.2. The product was believed to have 3S stereochemistry based on the starting material.

### Example 45

### 1-(2-fluoro-4-[(methoxycarbonyl)amino]phenyl)piperidin-3-yl 2-oxa-6-azatricyclo[3.3.1.1(3,7)]decane-6-carboxylate

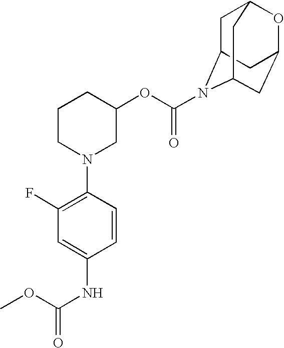

To a mixture of 1-(4-amino-2-fluorophenyl)piperidin-3-yl-2-oxa-6-azatricyclo[3.3.1.1(3,7)]decane-6-carboxylate (0.0200 g, 0.0000534 mol) and 1.00 M of sodium hydroxide in water (0.107 mL) in methylene chloride (0.500 mL, 0.00780 mol) was added methyl chloroformate (6.1903 μL, 8.0117E-5 mol). The reaction mixture was stirred at rt for 30 min. After the methylene chloride was removed, the residue was purified directly in RP-HPLC to give the desire product (20 mg, 87%). LCMS (M+H) 434.2. The product was believed to have 3S stereochemistry based on the starting material.

### Example 46

### 1-4-[(ethoxycarbonyl)amino]-2-fluorophenylpiperidin-3-yl 2-oxa-6-azatricyclo[3.3.1.1(3,7)1decane-6-carboxylate

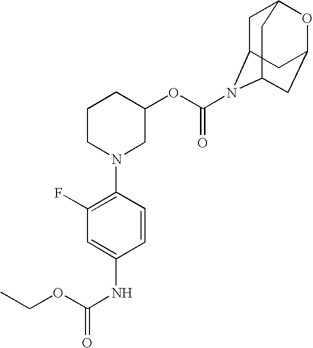

This compound was prepared using procedures analogous to those for Example 45. LCMS (M+H): 448.2.

### Example 47

### 1-(2-fluoro-4-[(propoxycarbonyl)amino]phenyl)piperidin-3-yl 2-oxa-6-azatricyclo[3.3.1.1(3,7)]decane-6-carboxylate

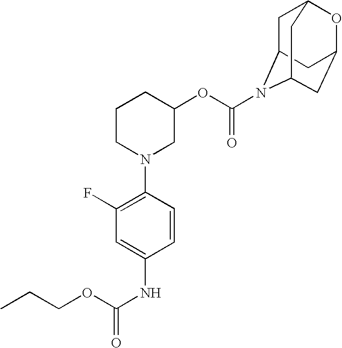

This compound was prepared using procedures analogous to those for Example 45. LCMS (M+H): 462.2.

### Example 48

### 1-(2-fluoro-4-[(isopropoxycarbonyl)amino]phenyl)piperidin-3-yl 2-oxa-6-azatricyclo[3.3.1.1(3,7)]decane-6-carboxylate

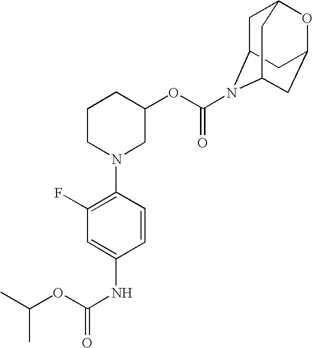

This compound was prepared using procedures analogous to those for Example 45. LCMS (M+H): 462.3.

### Example 49

### 1-[2-fluoro-4-(isobutyrylamino)phenyl]piperidin-3-yl 2-oxa-6-azatricyclo[3.3.1.1(3,7)]decane-6-carboxylate

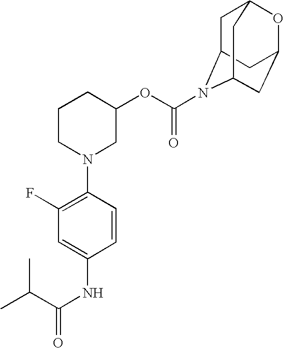

This compound was prepared using procedures analogous to those for Example 45. LCMS (M+H): 446.2.

### Example 50

### 1-(4-bromo-2-fluorophenyl)piperidin-3-yl-3-hydroxy-8-azabicyclo[3.2.1]octane-8-carboxylate

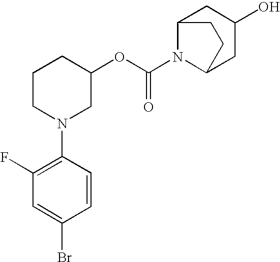

### Step 1. tert-butyl 3-[(3-oxo-8-azabicyclo[3.2.1]oct-8-yl)carbonyl]aminopiperidine-1-carboxylate

To a mixture of tert-butyl 3-[(4-nitrophenoxy)carbonyl]aminopiperidine-1-carboxylate (2.00 g. 0.00547 mol) and 8-azabicyclo[3.2.1]octan-3-one hydrochloride (0.804 g, 0.00498 mol) in acetonitrile (40.72 mL, 0.7796 mol) was added triethylamine (2.08 mL, 0.0149 mol). The reaction mixture was stirred at rt overnight, and then diluted with methylene chloride, washed with 1 N NaOH and brine respectively, dried, and concentrated. The residue was purified on silica gel, eluting with 0 to 100% EtOAc in hexane, to give the desired product 1.63 g, 93%). LCMS (M-Boc+H) 252.2.

### Step 2. piperidin-3-yl-3-hydroxy-8-azabicyclo[3.2.1]octane-8-carboxylate hydrochloride

tert-Butyl 3-[(3-oxo-8-azabicyclo[3.2.1]oct-8-yl)-carbonyl]-aminopiperidine-1-carboxylate (2.60 g, 0.00740 mol) was dissolved in tetrahydrofuran (51 mL, 0.63 mol) and cooled to −72° C. (internal temp). To the mixture was added 1.0 M of diisobutylaluminum hydride in hexane (11 mL) dropwise over 37 min, and the reaction temperature was kept below −63° C. The mixture was then stirred at less than −70° C. for 3.5 hours; and LCMS showed a single alcohol product. Then stirring of the mixture was continued at low temperature for 1 hour and the mixture was then quenched with water (0.2 mL). The cold bath was removed and the reaction mixture was allowed to warm to −30° C., and more water (0.2 mL) was added. After reaching −20° C., bubbling ceased. Additional 0.4 ml of water was added dropwise. The reaction mixture was warmed to 0° C.; then transferred to a separatory funnel. Then mixture was diluted with EtOAc and water, and 1 M sodium potassium tartrate was added to break up the emulsion/gel. The organic layer was separated from the aqueous layer and the organic layer was washed with 1M sodium potassium tartrate aqueous solution (3×) and water. To the combined aqueous layer was added solid tartrate until the solution was clarified. The aqueous solution was washed with EtOAc. The combine organic layer was dried (over Na₂SO₄), filtered, evaporated to give a white solid. LCMS (M+H) 354.3. The crude tert-butyl 3-([3-hydroxy-8-azabicyclo[3.2.1]oct-8-yl]carbonylamino)piperidine-1-carboxylate (2.32 g, 88.72%) was treated with 4 N HCl in dioxane to generate the corresponding HCl salt.

### Step 3. 1-(4-bromo-2-fluorophenyl)piperidin-3-yl-3-hydroxy-8-azabicyclo[3.2.1]octane-8-carboxylate

A mixture of piperidin-3-yl-3-hydroxy-8-azabicyclo[3.2.1]octane-8-carboxylate hydrochloride (1.67 g, 0.00574 mol), 4-bromo-2-fluoro-1-iodobenzene (2.07 g, 0.00689 mol), copper(I) iodide (0.11 g, 0.00057 mol), potassium phosphate (3.66 g, 0.0172 mol) and 1,2-ethanediol (0.640 mL, 0.0115 mol) in 1-butanol (5.63 mL, 0.0616 mol) was heated at 100° C. under nitrogen for 2 days. The reaction mixture was treated with water, and then extracted with ether. The organic layers were combined, washed with water and brine respectively, dried and evaporated to dryness. The residue was purified on silica gel, eluting with 0 to 50% EtOAc in hexane, to give the desired product (1.98 g, 80.68%). LCMS (M+H) 427.1. The product was believed to have 3S stereochemistry and 3-endo configuration based on the starting materials.

### Example 51

### 1-[2-fluoro-4-(2-oxopyrrolidin-1-yl)phenyl]piperidin-3-yl 2-oxa-6-azatricyclo[3.3.1.1(3,7)]decane-6-carboxylate

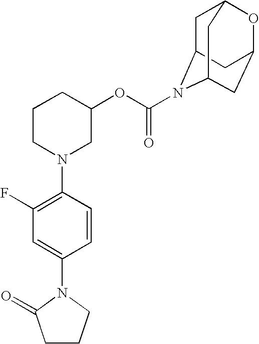

To a mixture of I-(4-amino-2-fluorophenyl)piperidin-3-yl 2-oxa-6-azatricyclo[3.3.1.1(3,7)]decane-6-carboxylate (20.0 mg, 0.0000533 mol) and 4-dimethylaminopyridine (9.762 mg, 7.991E-5 mol) in tetrahydrofuran (0.49 mL, 0.0060 mol) was added 4-bromobutanoyl chloride (0.00771 mL, 0.0000666 mol). The mixture was stirred at rt for 1 h, then treated with 1.00 M of potassium tert-butoxide in tetrahydrofuran (0.213 mL) at rt for 2 h, and then evaporated to dryness. The residue was neutralized with diluted HCl, then purified on RP-HPLC to give the product (20 mg, 84.65%). LCMS (M+H) 444.1. The product was believed to have 3S stereochemistry based on the starting material.

### Example 52

### 1-[2-fluoro-4-(2-oxo-1,3-oxazolidin-3-yl)phenyl]piperidin-3-yl 2-oxa-6-azatricyclo[3.3.1.1(3,7)]decane-6-carboxylate

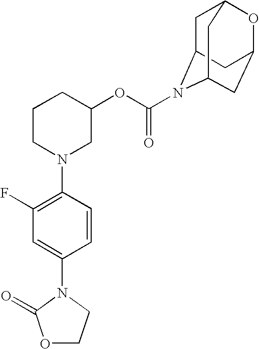

To a mixture of 1-(4-amino-2-fluorophenyl)piperidin-3-yl-2-oxa-6-azatricyclo[3.3.1.1(3,7)]decane-6-carboxylate (20.0 mg, 0.0000533 mol) and 4-dimethylaminopyridine (9.762 mg, 7.991E-5 mol) in tetrahydrofuran (0.49 mL, 0.0060 mol) was added carbonochloridic acid 2-chloroethyl ester (0.00688 mL, 0.0000666 mol). The mixture was stirred at rt for 1 h, then treated with 1.00 M of potassium tert-butoxide in tetrahydrofuran (0.213 mL) at rt for 2 h, and then evaporated to dryness. The residue was neutralized with diluted HCl, and purified on RP-HPLC to give the product (15 mg, 63.21%). LCMS (M+H) 446.2.

### Example 53

### 1-[2-fluoro-4-(2-oxo-1,3-oxazinan-3-yl)phenyl]piperidin-3-yl 2-oxa-6-azatricyclo[3.3.1.1(3,7)]decane-6-carboxylate

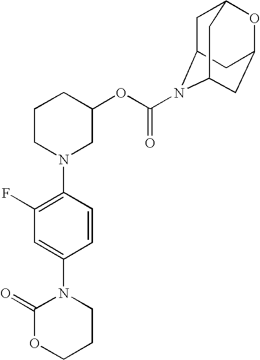

To a mixture of 1-(4-amino-2-fluorophenyl)piperidin-3-yl-2-oxa-6-azatricyclo-[3.3.1.1(3,7)]decane-6-carboxylate (20.0 mg, 0.0000533 mol) and 4-dimethylaminopyridine (9.762 mg, 7.991E-5 mol) in tetrahydrofuran (0.49 mL, 0.0060 mol) was added 3-chloropropyl chloridocarbonate (0.00803 mL, 0.0000666 mol). The mixture was stirred at rt for 1 h, then treated with 1.00 M of potassium tert-butoxide in tetrahydrofuran (0.213 mL) at rt for 2 h, and then evaporated to dryness. The residue was neutralized with diluted HCl, and then purified on RP-HPLC to give the product (14 mg, 57.19%). LCMS (M+H) 460.2. The product was believed to have 3S stereochemistry based on the starting materials.

### Example 54

### 1-[2-fluoro-4-(2-oxopiperidin-1-yl)phenyl]piperidin-3-yl 2-oxa-6-azatricyclo[3.3.1.1(3,7)]decane-6-carboxylate

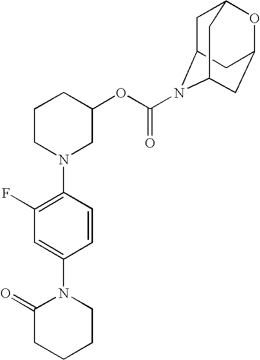

To a mixture of 1-(4-amino-2-fluorophenyl)piperidin-3-yl 2-oxa-6-azatricyclo[3.3.1.1(3,7)]decane-6-carboxylate (20.0 mg, 0.0000533 mol) and 4-dimethylaminopyridine (9.762 mg, 7.991E-5 mol) in tetrahydrofuran (0.33 mL, 0.0041 mol) was added 5-bromovaleryl chloride (0.00891 mL, 0.0000666 mol). The mixture was stirred at rt for 1 h, then treated with 1.00 M of potassium tert-butoxide in tetrahydrofuran (0.213 mL) at rt for 2 h, and then evaporated to dryness. The residue was neutralized with diluted HCl, and then purified on RP-HPLC to give the product (22 mg, 90.26%). LCMS (M+H) 458.3. The product was believed to have 3S stereochemistry based on the starting materials.

### Example 55

### 1-(2-fluoro-4-[(isobutoxycarbonyl)amino]phenyl)piperidin-3-yl 2-oxa-6-azatricyclo[3.3.1.1(3,7)]decane-6-carboxylate

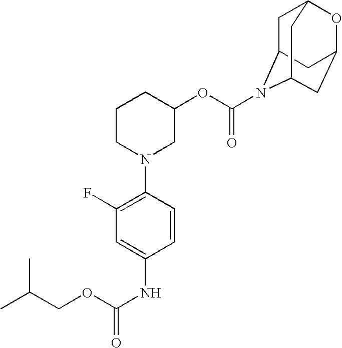

This compound was prepared using procedures analogous to those for Example 45. LCMS (M+H): 476.3.

### Example 56

### 1-(2-fluorophenyl)piperidin-3-yl-3-hydroxy-8-azabicyclo[3.2.1]octane-8-carboxylate

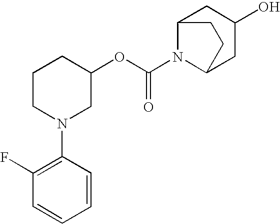

A mixture of 1-(4-bromo-2-fluorophenyl)piperidin-3-yl-3-hydroxy-8-azabicyclo[3.2.1]octane-8-carboxylate (0.010 g, 0.000023 mol) in 0.5 mL of MeOH was hydrogenated in the presence of 10% Pd/C, under a hydrogen balloon for 2 h. After the catalyst was filtered off, the filtrate was evaporated to dryness to give the desired product (8 mg, 98.12%). LCMS (M+H) 349.2. The product was believed to have 3S stereochemistry based on the starting materials.

### Example 57

### 1-(2-fluoro-4-6-[(methylamino)carbonyl]pyridin-3-ylphenyl)piperidin-3-yl-3-hydroxy-8-azabicyclo[3.2.1]octane-8-carboxylate

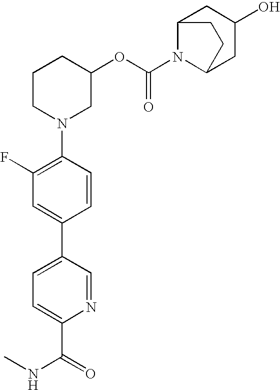

A mixture of 1-(4-bromo-2-fluorophenyl)piperidin-3-yl-3-hydroxy-8-azabicyclo[3.2.1]octane-8-carboxylate (25.0 mg, 0.0000585 mol), N-methyl-5-(4,4,5,5-tetramethyl-1,3,2-dioxaborolan-2-yl)pyridine-2-carboxamide (23.0 mg, 0.0000878 mol) and potassium carbonate (24.2 mg, 0.000176 mol) in N,N-dimethylformamide (0.50 mL, 0.0064 mol) was purged with nitrogen for 5 min. After [1,1′-bis(diphenylphosphino)ferrocene]dichloropalladium(II) complex with dichloromethane (1:1) (7.17 mg, 8.78E-6 mol) was added, the resulting mixture was heated at 120° C. for 4 h. The reaction mixture was diluted with AcCN and water, filtered through a 0.3 U membrane. The filtration was applied on RP-HPLC to generate the desired product (21 mg, 74.5%). LCMS (M+H) 483.2. The product was believed to have 3S stereochemistry and 3-endo configuration based on the starting materials.

### Example 58

### 1-(2-fluoro-4-pyridin-3-ylphenyl)piperidin-3-yl-3-hydroxy-8-azabicyclo[3.2.1]octane-8-carboxylate

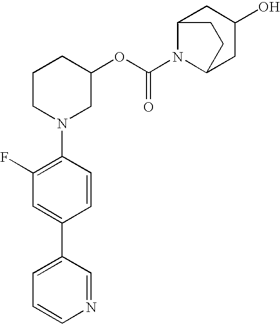

This compound was prepared using procedures analogous to those for Example 57. LCMS (M+H): 426.2.

### Example 59

### 1-(2-fluoro-4-pyridin-4-ylphenyl)piperidin-3-yl-3-hydroxy-8-azabicyclo[3.2.1]octane-8-carboxylate

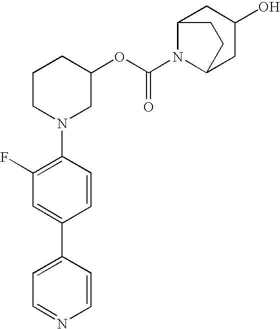

This compound was prepared using procedures analogous to those for Example 57. LCMS (M+H): 426.2.

### Example 60

### 1-(2-fluoro-4-pyrimidin-5-ylphenyl)piperidin-3-yl-3-hydroxy-8-azabicyclo[3.2.1]octane-8-carboxylate

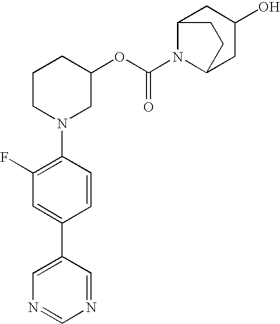

This compound was prepared using procedures analogous to those for Example 57. LCMS (M+H): 427.2.

### Example 61

### 1-[2-fluoro-4-(1-methyl-1H-pyrazol-4-yl)phenyl]piperidin-3-yl-3-hydroxy-8-azabicyclo[3.2.1]octane-8-carboxylate

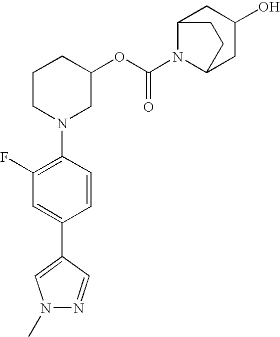

This compound was prepared using procedures analogous to those for Example 57. LCMS (M+H): 429.2.

### Example 62

### 1-4′-[(cyclopropylamino)carbonyl]-3-fluorobiphenyl-4-ylpiperidin-3-yl-3-hydroxy-8-azabicyclo[3.2.1]octane-8-carboxylate

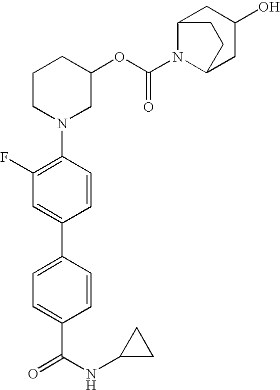

This compound was prepared using procedures analogous to those for Example 57. LCMS (M+H): 508.2.

### Example 63

### 1-(4-(6-[(dimethylamino)carbonyl]pyridin-3-yl)-2-fluorophenyl)piperidin-3-yl-3-hydroxy-8-azabicyclo[3.2.1]octane-8-carboxylate

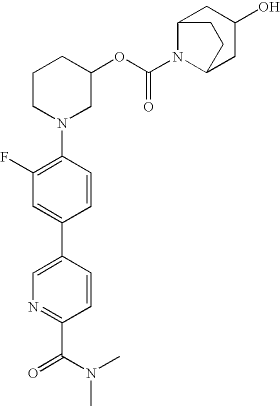

This compound was prepared using procedures analogous to those for Example 57. LCMS (M+H): 497.2.

### Example 64

### 1-(4-(6-[(ethylamino)carbonyl]pyridin-3-yl)-2-fluorophenyl)piperidin-3-yl-3-hydroxy-8-azabicyclo[3.2.1]octane-8-carboxylate

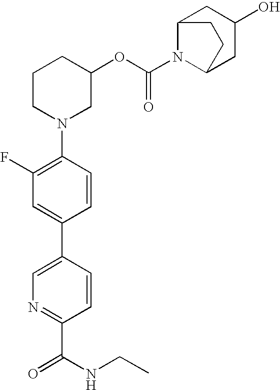

This compound was prepared using procedures analogous to those for Example 57. LCMS (M+H): 497.2.

### Example 65

### 1-(4-(6-[(diethylamino)carbonyl]pyridin-3-yl)-2-fluorophenyl)piperidin-3-yl-3-hydroxy-8-azabicyclo[3.2.1]octane-8-carboxylate

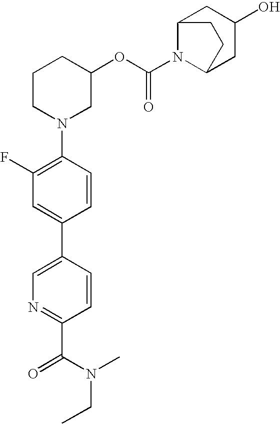

This compound was prepared using procedures analogous to those for Example 57. LCMS (M+H): 525.3.

### Example 66

### 1-[4′-(aminocarbonyl)-3-fluorobiphenyl-4-yl] piperidin-3-yl-3-hydroxy-8-azabicyclo[3.2.1]octane-8-carboxylate

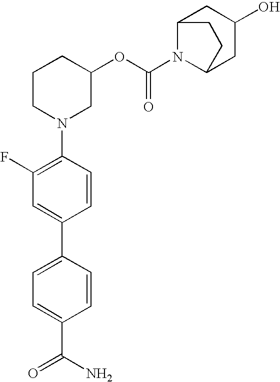

This compound was prepared using procedures analogous to those for Example 57. LCMS (M+H): 468.2.

### Example 67

### 3,5-difluoro-4-(3-2-[3-hydroxy-8-azabicyclo[3.2.1]oct-8-yl]-2-oxoethylpiperidin-1-yl)benzonitrile

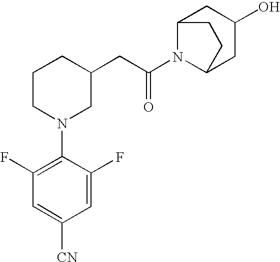

### Step 1. 8-(piperidin-3-ylacetyl)-8-azabicyclo[3.2.1]octan-3-ol hydrochloride

To a mixture of [1-(tert-butoxycarbonyl)piperidin-3-yl]acetic acid (148.7 mg, 0.0006111 mol) and (3-endo)-8-azabicyclo[3.2.1]octan-3-ol hydrochloride (100.0 mg, 0.0006111 mol) in N,N-dimethylformamide (2.00 mL, 0.0258 mol) was added benzotriazol-1-yloxytris(dimethylamino)phosphonium hexafluorophosphate (297.3 mg, 0.0006722 mol). The reaction mixture was stirred at rt for 15 min, then treated with N,N-diisopropylethylamine (0.2661 mL, 0.001528 mol) at rt for another 2 h. LCMS indicated the formation of the coupled product, (M+H) 353.2. The mixture was diluted with water, then extracted with EtOAc. The combined organic layers were washed with aq. sodium bicarbonate, water, and brine successively, dried, and concentrated to dryness. The residue was treated with hydrogen chloride in 1,4-dioxane (4.00 M, 3.06 mL) at rt for 4 h. After it was concentrated to dryness, the resulting HCl salt was used directly in next step (170 mg, 96%). LCMS (M+H) 253.2.

### Step 2. 3,5-difluoro-4-(3-2-[3-hydroxy-8-azabicyclo[3.2.1]oct-8-yl]-2-oxoethylpiperidin-1-yl)benzonitrile

A mixture of 8-(piperidin-3-ylacetyl)-8-azabicyclo[3.2.1]octan-3-ol hydrochloride (0.035 g, 0.00012 mol), 3,4,5-trifluorobenzonitrile (0.0209 g, 0.000133 mol) and potassium carbonate (0.0419 g, 0.000303 mol) in N,N-dimethylformamide (0.700 mL, 0.00904 mol) was heated at 100° C. overnight. After quenched with water, the mixture was extracted with EtOAc. The organic layers were combined, washed with water and brine successively, dried, and evaporated to dryness. The residue was purified on RP-HPLC to give the desired product (36 mg 77%). LCMS (M+H) 390.2. The product was believed to have a 3-endo configuration based on the starting materials.

### Example 68

### 8-[1-(2-fluoro-4-nitrophenyl)piperidin-3-yl]acetyl-8-azabicyclo[3.2.1]octan-3-ol

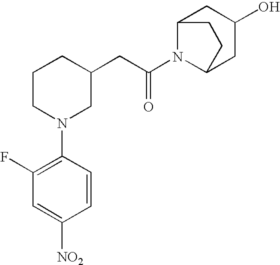

A mixture of (3-endo)-8-(piperidin-3-ylacetyl)-8-azabicyclo[3.2.1]octan-3-ol hydrochloride (0.280 g, 0.000969 mol), 3,4-difluoronitrobenzene (0.170 g, 0.00107 mol) and potassium carbonate (0.335 g, 0.00242 mol) in N,N-dimethylformamide (5.60 mL, 0.0723 mol) was heated at 100° C. overnight. After quenching with water, the mixture was extracted with EtOAc. The organic layers were combined, washed with water and brine successively, dried, and evaporated to dryness. The residue was purified on RP-HPLC to give the desired product (349 mg, 92%). LCMS (M+H) 392.2. The product was believed to have a 3-endo configuration based on the starting materials.

### Example 69

### 8-[1-(4-amino-2-fluorophenyl)piperidin-3-yl]acetyl-8-azabicyclo[3.2.1]octan-3-ol

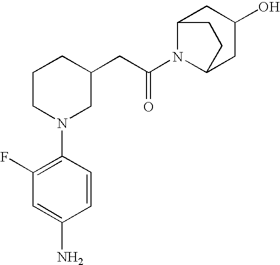

A mixture of 8-[1-(2-fluoro-4-nitrophenyl)piperidin-3-yl]acetyl-8-azabicyclo[3.2.1]octan-3-ol (0.36 g, 0.00092 mol) in 5 mL of MeOH was hydrogenated in the presence of 10% Pd/C, under a hydrogen balloon at rt for 2 h. After the mixture was filtered and the filtrated was evaporated to dryness. The residue was used directly in next step. An analytically pure sample was obtained by RP-HPLC. LCMS (M+H) 362.2. The product was believed to have a 3-endo configuration based on the starting materials.

### Example 70

### methyl [3-fluoro-4-(3-2-[3-hydroxy-8-azabicyclo[3.2.1]oct-8-yl]-2-oxoethylpiperidin-1-yl)phenyl]carbamate

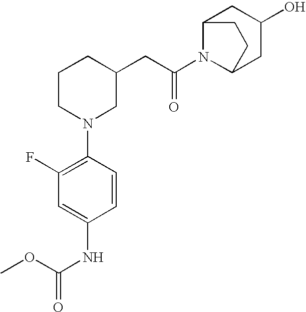

To a mixture of 8-[1-(4-amino-2-fluorophenyl)piperidin-3-yl]acetyl-8-azabicyclo[3.2.1]octan-3-ol (0.030 g, 0.000083 mol) and a solution of sodium hydroxide in water (1.00 M, 0.166 mL) in methylene chloride (0.500 mL, 0.00780 mol) was added methyl chloroformate (0.0118 g, 0.000124 mol). The reaction mixture was stirred at rt for 30 min, and methylene chloride was stripped off. The residue was purified on RP-HPLC to give the desired product (32 mg, 92%). LCMS (M+H) 420.2. The product was believed to have a 3-endo configuration based on the starting materials.

### Example 71

### ethyl [3-fluoro-4-(3-2-[3-hydroxy-8-azabicyclo[3.2.1]oct-8-yl]-2-oxoethylpiperidin-1-yl)phenyl]carbamate

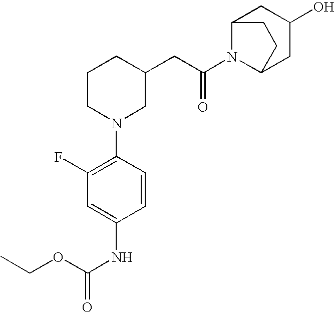

This compound was prepared using procedures analogous to those for Example 70. LCMS (M+H): 434.3.

### Example 72

### propyl [3-fluoro-4-(3-2-[3-hydroxy-8-azabicyclo[3.2.1]oct-8-yl]-2-oxoethylpiperidin-1-yl)phenyl]carbamate

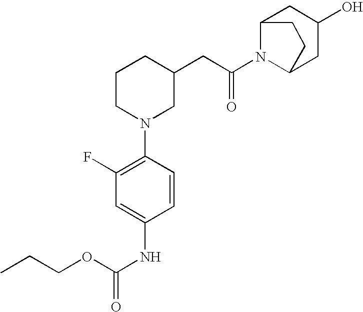

This compound was prepared using procedures analogous to those for Example 70. LCMS (M+H): 448.3.

### Example 73

### isopropyl [3-fluoro-4-(3-2-[3-hydroxy-8-azabicyclo[13.2.1]oct-8-yl]-2-oxoethylpiperidin-1-yl)phenyl]carbamate

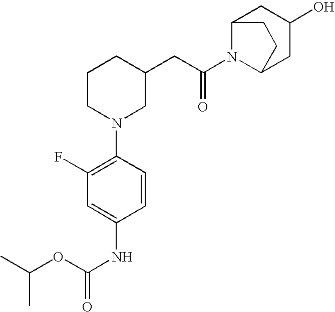

This compound was prepared using procedures analogous those for Example 70. LCMS (M+H): 448.3.

### Example 74

### isobutyl [3-fluoro-4-(3-2-[3-hydroxy-8-azabicyclo[3.2.1]oct-8-yl]-2-oxoethylpiperidin-1-yl)phenyl]carbamate

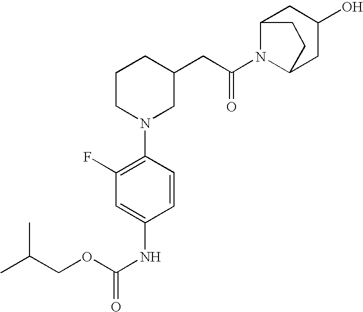

This compound was prepared using procedures analogous to those for Example 70. LCMS (M+H): 462.3.

### Example 75

### 3-fluoro-4-(3-(2-[3-hydroxy-8-azabicyclo[3.2.1]oct-8-yl -2-oxoethyl)piperidin-1-yl)benzonitrile

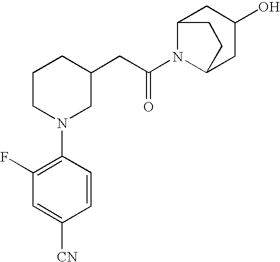

### Step 1. 8-[piperidin-3-ylacetyl]-8-azabicyclo[3.2.1]octan-3-ol hydrochloride

To a mixture of [(3R)-1-(tert-butoxycarbonyl)piperidin-3-yl]acetic acid (1.000 g, 0.004110 mol) and (3-endo)-8-azabicyclo[3.2.1]octan-3-ol hydrochloride (0.6726 g, 0.004110 mol) in N,N-dimethylformamide (13.4 mL, 0.174 mol) was added benzotriazol-1-yloxytris(dimethylamino)phosphonium hexafluorophosphate (2.000 g, 0.004521 mol). The reaction mixture was stirred at rt for 15 min, then treated with N,N-diisopropylethylamine (1.790 mL, 0.01028 mol) at rt for another 2 h. LCMS indicated the formation of the coupled product, (M+H) 353.2. The mixture was diluted with water, and extracted with EtOAc. The combined organic layers were washed with aq. sodium bicarbonate, water and brine successively, dried, and evaporated to dryness. The residue was treated with hydrogen chloride in 1,4-dioxane (4.00 M, 20.55 mL) at rt for 4 h. After it was evaporated to dryness, the resulting HCl salt was used directly in next step (1.19 g, 99.91%). LCMS (M+H) 253.2.

### Step 2. 3-fluoro-4-(3-(2-[3-hydroxy-8-azabicyclo[3.2.1]oct-8-yl]-2-oxoethyl)piperidin-1-yl)benzonitrile

A mixture of 8-[piperidin-3-ylacetyl]-8-azabicyclo[3.2.1]octan-3-ol hydrochloride (0.020 g, 0.000069 mol), 3,4-difluorobenzonitrile (0.0106 g, 0.0000762 mol) and potassium carbonate (0.0239 g, 0.000173 mol) in N,N-dimethylformamide (0.400 mL, 0.00516 mol) was heated at 120° C. overnight. After quenching with water, the mixture was extracted with EtOAc. The organic layers were combined, washed with water and brine successively, dried, and evaporated to dryness. The residue was purified on RP-HPLC to give the desired product (21 mg, 81.64%). LCMS (M+H): 372.2. The product was believed to have 3R stereochemistry and a 3-endo configuration based on the starting materials.

### Example 76

### 8-[1-(5-chloro-3-fluoropyridin-2-yl)piperidin-3-yl]acetyl-8-azabicyclo13.2.1]octan-3-ol

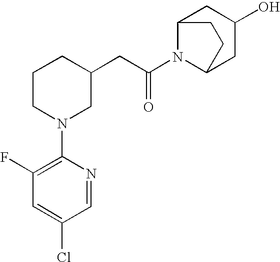

A mixture of 8-[piperidin-3-ylacetyl]-8-azabicyclo[3.2.1]octan-3-ol hydrochloride (27.4 mg, 0.0000950 mol), 5-chloro-2,3-difluoropyridine (0.0156 g, 0.000104 mol) and N,N-diisopropylethylamine (0.0496 mL, 0.000285 mol) in N-methylpyrrolidinone (0.500 mL, 0.00518 mol) was microwaved at 180° C. for 20 min. The resultant mixture was applied on RP-HPLC to give the desired product (16 mg 44%. LCMS (M+H) 382.2.

### Example 77

### 8-(1-[4-(trifluoromethyl)pyridin-2-yl]piperidin-3-ylacetyl)-8-azabicyclo[13.2.1]octan-3-ol

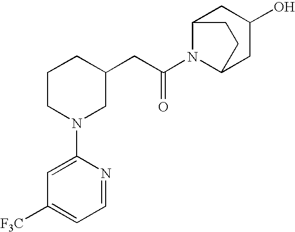

This compound was prepared using procedures analogous to those for Example 76. LCMS (M+H) 398.2.

### Example 78

### 8-[1-(3-chloropyridin-2-yl)piperidin-3-yl]acetyl-8-azabicyclo[13.2.1]octan-3-ol

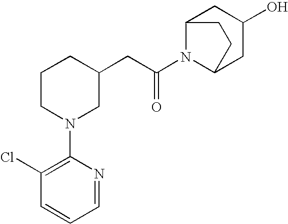

This compound was prepared using procedures analogous to those for Example 76. LCMS (M+H) 364.2.

### Example 79

### 8-(1-[3-chloro-5-(trifluoromethyl)pyridin-2-yl]piperidin-3-ylacetyl)-8-azabicyclo[13.2.1]octan-3-ol

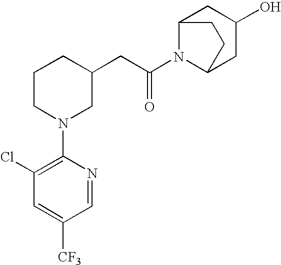

This compound was prepared using procedures analogous to those for Example 76. LCMS (M+H) 432.1.

### Example 80

### 1-(2-fluoro-4-methylphenyl)piperidin-3-yl-methyl(tetrahydro-2H-pyran-4-yl)carbamate

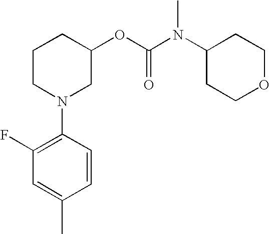

To a mixture of 1-(2-fluoro-4-methylphenyl)piperidin-3-ol (30.0 mg, 0.000143 mol) (see Ex. 29) and p-nitrophenyl chloroformate (30.3 mg, 0.000150 mol) in methylene chloride (0.500 mL, 0.00780 mol) was added triethylamine (0.0999 mL, 0.000717 mol). The mixture was stirred at rt for 30 min, then treated with N-methyltetrahydro-2H-pyran-4-amine hydrochloride (23.9 mg, 0.000158 mol) at rt overnight. After evaporation to dryness, the resultant mixture was purified on RP-HPLC to give the desired product (31 mg, 59%). LCMS (M+H) 351.2. The product was believed to have 3S stereochemistry based on the starting materials.

### Example 81

### 1-(2-fluoro-4-methylphenyl)piperidin-3-yl-3-methylmorpholine-4-carboxylate

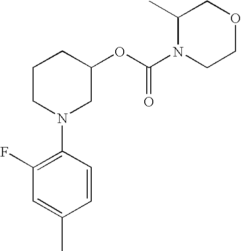

This compound was prepared using procedures analogous to those for Example 80. LCMS (M+H) 337.2.

### Example 82

### 1-(2,4-difluorophenyl)piperidin-3-yl-3-methylmorpholine-4-carboxylate

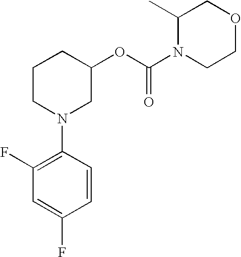

This compound was prepared using procedures analogous to those for Example 80. LCMS (M+H) 341.2.

### Example 83

### 1-(2,4-difluorophenyl)piperidin-3-yl-methyl-(tetrahydro-2H-pyran-4-yl)carbamate

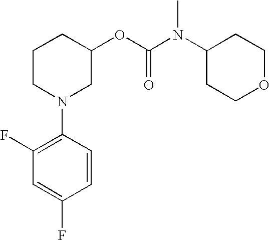

This compound was prepared using procedures analogous to those for Example 80. LCMS (M+H) 355.2.

### Example 84

### 1-(2,4-difluorophenyl)piperidin-3-yl-(4-hydroxycyclohexyl)methylcarbamate

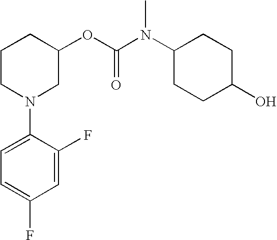

This compound was prepared using procedures analogous to those for Example 80. LCMS (M+H) 369.1.

### Example 85

### 1-(2-fluoro-4-methylphenyl)piperidin-3-yl-(4-hydroxycyclohexyl)-methylcarbamate

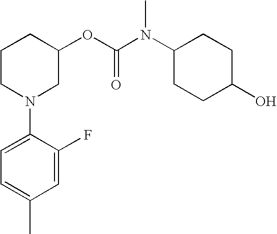

This compound was prepared using procedures analogous to those for Example 80. LCMS (M+H) 365.2.

### Example A

Enzymatic Assay of 11βHSD1

All in vitro assays were performed with clarified lysates as the source of 11βHSD1 activity. HEK-293 transient transfectants expressing an epitope-tagged version of full-length human 11βHSD1 were harvested by centrifugation. Roughly 2×10⁷ cells were resuspended in 40 mL of lysis buffer (25 mM Tris-HCl, pH 7.5, 0.1 M NaCl, 1 mM MgCl₂ and 250 mM sucrose) and lysed in a microfluidizer. Lysates were clarified by centrifugation and the supernatants were aliquoted and frozen.

Inhibition of 11βHSD1 by test compounds was assessed in vitro by a Scintillation Proximity Assay (SPA). Dry test compounds were dissolved at 5 mM in DMSO. These were diluted in DMSO to suitable concentrations for the SPA assay. 0.8 μL of 2-fold serial dilutions of compounds were dotted on 384 well plates in DMSO such that 3 logs of compound concentration were covered. 20 μL of clarified lysate was added to each well. Reactions were initiated by addition of 20 μL of substrate-cofactor mix in assay buffer (25 mM Tris-HCl, pH 7.5, 0.1 M NaCl, 1 mM MgCl₂) to final concentrations of 400 μM NADPH, 25 nM ³H-cortisone and 0.007% Triton X-100. Plates were incubated at 37° C. for one hour. Reactions were quenched by addition of 40 μL of anti-mouse coated SPA beads that had been pre-incubated with 10 μM carbenoxolone and a cortisol-specific monoclonal antibody. Quenched plates were incubated for a minimum of 30 minutes at RT prior to reading on a Topcount scintillation counter. Controls with no lysate, inhibited lysate, and with no mAb were run routinely. Roughly 30% of input cortisone is reduced by 11βHSD1 in the uninhibited reaction under these conditions.

Test compounds having an IC₅₀ value less than about 20 μM according to this assay were considered active.

### Example B

Cell-Based Assays for HSD Activity

Peripheral blood mononuclear cells (PBMCs) were isolated from normal human volunteers by Ficoll density centrifugation. Cells were plated at 4×10⁵ cells/well in 200 μL of AIM V (Gibco-BRL) media in 96 well plates. The cells were stimulated overnight with 50 ng/ml recombinant human IL-4 (R&D Systems). The following morning, 200 nM cortisone (Sigma) was added in the presence or absence of various concentrations of compound. The cells were incubated for 48 hours and then supernatants were harvested. Conversion of cortisone to cortisol was determined by a commercially available ELISA (Assay Design).

Test compounds having an IC₅₀ value less than about 20 μM according to this assay were considered active.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula Ia or Ib: 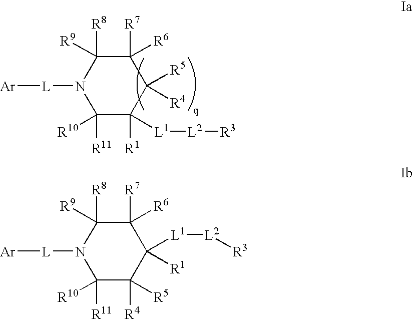
or pharmaceutically acceptable salt or prodrug thereof, wherein:
L is absent, S(O)₂, S(O), S, S(O)₂NR², C(O), C(O)O, C(O)O—(C₁₋₃ alkylene), or C(O)NR²;
L¹ is O, CH₂, or NR^{N};
L² is CO or S(O)₂;
provided that when L¹ is NR^{N}, L² is SO₂;
R^{N} is H, C ₁₋₆ alkyl, aryl, cycloalkyl, heteroaryl or heterocycloalkyl;
Ar is aryl or heteroaryl, each optionally substituted by 1, 2, 3, 4 or 5 —W—X—Y-Z;
R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹⁴;
R² is H or C ₁₋₆ alkyl;
R³ is H, C₁₋₆ alkyl, aryl, cycloalkyl, heteroaryl or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
or R³ is NR^{3a}R^{3b} or OR^{3c};
R^{3a} and R^{3b} are independently selected from H, C₁₋₆ alkyl, aryl, cycloalkyl, heteroaryl and heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
or R^{3a} and R^{3b} together with the N atom to which they are attached form a 4-14 membered heterocycloalkyl group which is optionally substituted by 1, 2 or 3 —W′—X′—Y′-Z′;
R^{3c} is H, C₁₋₆ alkyl, aryl, cycloalkyl, heteroaryl or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}. SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl is optionally substituted by 1, 2 or 3 R¹⁴;
or R¹ and R³ together with the carbon atoms to which they are attached and the intervening —NR²CO— moiety form a 4-14 membered heterocycloalkyl group which is optionally substituted by 1, 2 or 3 R¹⁴;
or R⁴ and R⁵ together with the carbon atom to which they are attached form a 3-14 membered cycloalkyl or heterocycloalkyl group which is optionally substituted by 1, 2 or 3 R¹⁴;
or R⁶ and R⁷ together with the carbon atom to which they are attached form a 3-14 membered cycloalkyl or heterocycloalkyl group which is optionally substituted by 1, 2 or 3 R¹⁴;
or R⁸ and R⁹ together with the carbon atom to which they are attached form a 3-14 membered cycloalkyl or heterocycloalkyl group which is optionally substituted by 1, 2 or 3 R¹⁴;
or R¹⁰ and R¹¹ together with the carbon atom to which they are attached form a 3-14 membered cycloalkyl or heterocycloalkyl group which is optionally substituted by 1, 2 or 3 R¹⁴;
or R⁴ and R⁶ together with the carbon atom to which they are attached form a 3-7 membered fused cycloalkyl group or 3-7 membered fused heterocycloalkyl group which is optionally substituted by 1, 2 or 3 R¹⁴;
or R⁶ and R⁸ together with the carbon atom to which they are attached form a 3-7 membered fused cycloalkyl group or 3-7 membered fused heterocycloalkyl group which is optionally substituted by 1,2or3R¹⁴;
each R¹⁴ is independently halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S()₂R^{b′}, or S(O)₂NR^{c′}R^{d′};
W, W′ and W″ are independently selected from absent, C₁₋₁₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of said C₁₋₆ alkylenyl, C₂₋₆ alkenylenyl, and C₂₋₆ alkynylenyl is optionally substituted by 1, 2 or 3 substituted independently selected from halo, OH, C₁₋₄ alkoxy, C₁₋₄ haloalkoxy, amino, C₁₋₄ alkylamino, and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of said C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₄ alkyl, C₁₋₄ haloalkyl, C₂₋₈ alkoxyalkyl, C₁₋₄ alkoxy, C₁₋₄ haloalkoxy, C₂₋₈ alkoxyalkoxy, cycloalkyl, heterocycloalkyl, C(O)OR^{a}, C(O)NR^{c}R^{d}, amino, C₁₋₄ alkylamino, and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl and C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;
Z, Z′ and Z″ are independently selected from H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ a cycloalkyl, heteroaryl, heterocycloalkyl, halosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, C(═NR^{g}NR^{c}R^{d}, NR^{c}C(═NR^{g})NR^{c}R^{d}, S(O)R^{b}, S(O)NR^{c}R^{d}, S(O)₂R^{b}, and S(O)₂NR^{c}R^{d} wherein each of said C₁₋₆ alkyl, C₂₋₆ alkenyl, C₂₋₆ alkynyl, aryl, cycloalkyl, heteroaryl, and heterocycloalkyl is optionally substituted by 1, 2 or 3 substituents independently selected from halo, C₁₋₆ alkyl, C₂₋₆ alkenyl, C₂₋₆ alkynyl, C₁₋₄ haloalkyl, aryl, cycloalkyl, heteroaryl, heterocycloalkyl, halosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, C(═NR^{g})NR^{c}R^{d}, NR^{c}C(═NR^{g})NR^{c}R^{d}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of said C₁₋₆ alkyl, C₁₋₆ haloalkyl, C₂₋₆ alkenyl, C₂₋₆ alkynyl, aryl, cycloalkyl, heteroaryl, and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, amino, halo, C₁₋₆ alkyl, C₁₋₆ haloalkyl, C₁₋₁₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{e} and R^{f} together with the N atom to which they are attached form a 4-, 5-, 6- or 7-membered heterocycloalkyl group;
R^{g} is H, CN, NO₂, C(O)NH₂, or C₁₋₆ alkyl; and
q is 0, 1 or 2;
with the provisos:
(a) when the compound has Formula Ia; q is l; L is C(O)CH₂; L¹ is CH₂; L² is S(O)₂; R⁴, R⁵, R⁶, R⁷, R⁸, R⁹, R¹⁰ and R¹¹ are each H; R³ is NR^{3a}R^{3b}; and R^{3a} and R^{3b} together with the N atom to which they are attached form an optionally substituted 4-14 membered heterocycloalkyl group, then R³ is other than piperidinyl substituted by heteroaryl wherein the heteroaryl is optionally substituted by arylalkyl;
(b) when the compound has Formula Ia, q is 0, L is C(O)CH₂, R³ is NR^{3a}R^{3b}, and R^{3a} and R^{3b} together with the N atom to which they are attached form an optionally substituted 4-14 membered heterocycloalkyl group, then Ar is other than optionally substituted aryl;
(c) when the compound has Formula Ia, q is 0, L is CO or S(O)₂, R³ is NR^{3a}R^{3b}, and R^{3a} and R^{3b} together with the N atom to which they are attached form an optionally substituted 4-14 membered heterocycloalkyl group, then each of R⁴, R⁵, R⁶, R⁷, R⁸, R⁹, R¹⁰ and R¹¹ is other than OC(O)R^{a′}, OC(O)OR^{b′}, C(O)OR^{b′} or OC(O)NR^{c′}R^{d′}; and
(d) when the compound has Formula Ia, q is 0, L is absent, R³is NR^{3a}R^{3b}, and R^{3a} and R^{3b} together with the N atom to which they are attached form an optionally substituted 4-14 membered heterocycloalkyl group, then R³ is other than optionally substituted piperazinyl or optionally substituted 3-oxo-piperazinyl.

2. The compound of claim 1, or pharmaceutically acceptable salt thereof, wherein L is S(O)₂.

3. The compound of claim 1, or pharmaceutically acceptable salt thereof, wherein L is absent.

4. The compound of claim 1, or pharmaceutically acceptable salt thereof, wherein L is CO.

5. The compound of claim 1, or pharmaceutically acceptable salt thereof, wherein L¹ is O and L² is CO.

6. The compound of claim 1, or pharmaceutically acceptable salt thereof, wherein L¹ is CH₂ and L² is CO.

7. The compound of claim 1, or pharmaceutically acceptable salt thereof, wherein L¹ is CH₂ and L² is S(O)₂.

8. The compound of claim 1, or pharmaceutically acceptable salt thereof, wherein L¹ is NH and L² is S(O)₂.

9. The compound of claim 1, or pharmaceutically acceptable salt thereof, wherein R¹ is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

10. The compound claim 1, or pharmaceutically acceptable salt thereof, wherein R¹ is H, C₁₋₆ alkyl, or C₁₋₆ haloalkyl.

11. The compound of claim 1, or pharmaceutically acceptable salt thereof, wherein R³ is NR^{3a}R^{3b}, and R^{3a} and R^{3b} together with the N atom to which they are attached form a 4-14 membered heterocycloalkyl group which is optionally substituted by 1, 2 or 3 —W′—X′—Y′-Z′.

12. The compound of claim 1, or pharmaceutically acceptable salt thereof, wherein R⁴, R⁵, R⁶, R⁷, R⁸, R⁹, R¹⁰ and R¹¹ are independently selected from H, NR^{c′}R^{d′}, NR^{c′}C(O)R^{a′}, NR^{c′}C(O)OR^{b′}, S(O)R^{a′}, S(O)N^{c′}R^{c′}R^{d′}, S(O)₂R^{a′}, S(O)₂NR^{c′}R^{d′}; SR^{b′}, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl.

13. The compound of claim 1, or pharmaceutically acceptable salt thereof, wherein R⁴, R⁵, R⁶, R⁷, R⁸, R⁹, R¹⁰ and R¹¹ are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl.

14. The compound of claim 1, or pharmaceutically acceptable salt thereof, wherein R⁴, R⁵, R⁶, R⁷, R⁸, R⁹, R¹⁰ and R¹¹ are independently selected from H, C ₁₆alkyl, C ₁₆haloalkyl, C₂₆alkenyl and C₂₋₆ alkynyl.

15. The compound of claim 1, or pharmaceutically acceptable salt thereof, wherein R⁴, R⁵, R⁶, R⁷, R⁸, R⁹, R¹⁰ and R¹¹ are independently selected from H, C₁₋₆ alkyl and C₁₋₆haloalkyl.

16. The compound of claim 1, or pharmaceutically acceptable salt thereof, wherein q is 0 or 1.

17. The compound of claim 1, or pharmaceutically acceptable salt thereof, wherein q is 1.

18. The compound of claim 1, or pharmaceutically acceptable salt thereof, wherein the compound has Formula II: 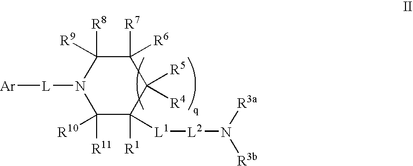
wherein R^{3a} and R^{3b} together with the N atom to which they are attached form a 4-14 membered heterocycloalkyl group which is optionally substituted by 1, 2 or 3 —W′—X′—Y′-Z′.

19. The compound of 18, or pharmaceutically acceptable salt thereof, wherein the ring-forming atoms of the heterocycloalkyl group are selected from N, C and O.

20. The compound of claim 18, or pharmaceutically acceptable salt thereof, wherein L is absent, S(O)₂ or CO.

21. The compound of claim 18, or pharmaceutically acceptable salt thereof, wherein q is 0 or 1.

22. The compound of claim 18, or pharmaceutically acceptable salt thereof, wherein the compound has Formula III: 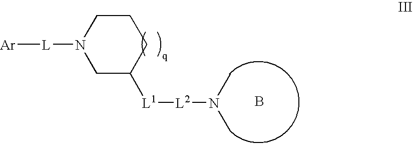
wherein ring B is a 4-14 membered heterocycloalkyl group which is optionally substituted by 1, 2 or 3 —W′—X′—Y′-Z′.

23. The compound of claim 22, or pharmaceutically acceptable salt thereof, wherein L is absent, S(O)₂ or CO.

24. The compound of claim 22, or pharmaceutically acceptable salt thereof, wherein the compound has Formula IVa, IVb, IVc, or IVd:

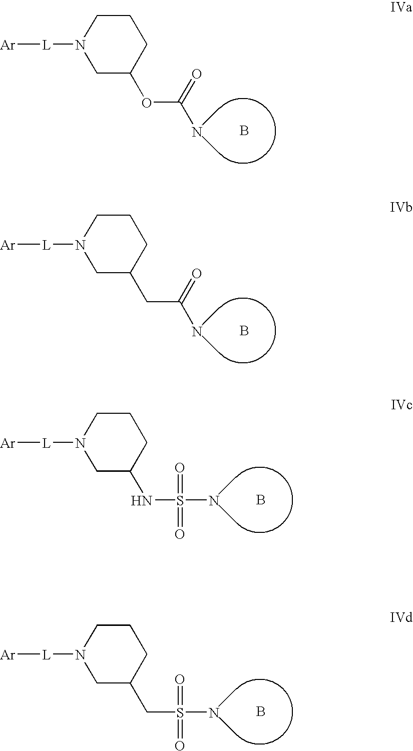

25. The compound of claim 24, or pharmaceutically acceptable salt thereof, wherein the ring-forming atoms of ring B are selected from N, C and O.

26. The compound of claim 24, or pharmaceutically acceptable salt thereof, wherein ring B is pyrrolidinyl, piperidinyl, morpholino, 8-azabicyclo[3.2.1 ]octan-8-yl, 9-azabicyclo[3.3.1 ]nonan-9-yl or 2-oxa-6-azatricyclo[3.3.1.1(3.7)]decan-6-yl, each optionally substituted by 1, 2 or 3 —W′—X′—Y′-Z′.

27. The compound of claim 24, or pharmaceutically acceptable salt thereof, wherein ring B is substituted by 1 OH.

28. The compound of claim 24, or pharmaceutically acceptable salt thereof, wherein the compound has Formula IVa or Formula IVb.

29. The compound of claim 1, or pharmaceutically acceptable salt thereof, wherein Ar is aryl optionally substituted by 1, 2, 3, 4 or 5 —W—X—Y-Z.

30. The compound of claim 1, or pharmaceutically acceptable salt thereof, wherein Ar is phenyl or naphthyl, each optionally substituted by 1, 2, 3, 4 or 5 —W—X—Y-Z.

31. The compound of claim 1, or pharmaceutically acceptable salt thereof, wherein Ar is phenyl or naphthyl, each optionally substituted by 1, 2, 3, 4 or 5 substituents independently selected from halo, CN, NO₂, C₁₋₄ alkoxy, heteroaryloxy, C₂₋₆ alkynyl, C, ₄ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected form halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

32. The compound of claim 1, or pharmaceutically acceptable salt thereof, wherein Ar is phenyl or naphthyl, each optionally substituted by 1, 2, 3, 4 or 5 substituents independently selected from halo, CN, NO₂, NR^{c}C(O)R^{d}, NR^{c}C(O)OR^{a}, NR^{c}R^{d}, C₁₋₆ alkyl, aryl and heteroaryl, wherein each of said aryl and heteroaryl is optionally substituted by 1, 2 or 3 substituents independently selected from C₁₋₆ alkyl and C(O)NR^{c}R^{d}.

33. The compound of claim 1, or pharmaceutically acceptable salt thereof, wherein Ar is heteroaryl optionally substituted by 1, 2, 3, 4 or 5 —W—X—Y-Z.

34. The compound of claim 1, or pharmaceutically acceptable salt thereof, wherein Ar is heteroaryl optionally substituted by 1, 2, 3, 4 or 5 substituents independently selected from halo, CN, NO₂, C₁₋₄ alkoxy, heteroaryloxy, C₂₋₆ alkynyl, C₁₋₄ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected from halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

35. The compound of claim 1, or pharmaceutically acceptable salt thereof, wherein Ar is pyridyl, pyrimidinyl, thienyl, thiazolyl, quinolinyl, 2,1,3-benzoxadiazolyl, isoquinolinyl or isoxazolyl, each optionally substituted by 1, 2, 3, 4 or 5 substituents independently selected from halo, CN, NO₂, C₁₋₄ alkoxy, heteroaryloxy, C₂₋₆ alkynyl, C₁₋₄ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected from halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

36. The compound of claim 1, or pharmaceutically acceptable salt thereof, wherein Ar is pyridyl optionally substituted by 1, 2, 3, 4 or 5 substituents independently selected from halo, CN, NO₂, C₁₋₄ alkoxy, heteroaryloxy, C₂₋₆ alkynyl, C₁₋₄ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected from halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

37. The compound of claim 1, or pharmaceutically acceptable salt thereof, wherein the compound has Formula Va, Vb or Vc: 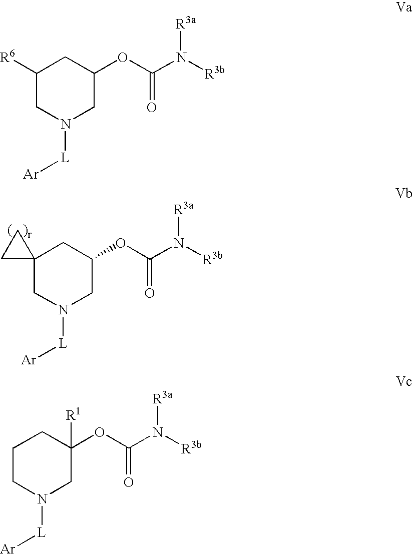
wherein:
r is 1, 2, 3, 4 or 5; and
R^{3a} and R^{3b} together with the N atom to which they are attached form a 4-14 membered heterocycloalkyl group which is optionally substituted by 1, 2 or 3 —W′—X′—Y′-Z′.

38. The compound of claim 1, or pharmaceutically acceptable salt thereof, wherein the compound has Formula Ia; L¹ is O; L² is CO; q is 1; R³ is NR^{3a}R^{3b}; R^{3a} is C₁₋₆alkyl; and R^{3b} is a 4-7 membered heterocycloalkyl group.

39. A compound selected from:
1-(1-naphthylsulfonyl)piperidin-3-yl piperidine-1-carboxylate;
1-(1-naphthylsulfonyl)piperidin-3-yl 4-hydroxypiperidine-1-carboxylate;
1-(1-naphthylsulfonyl)piperidin-3-yl-3-hydroxy-8-azabicyclo[3.2.1]octane-8-carboxylate;
1-(2-fluoro-4-nitrophenyl)piperidin-3-yl-3-hydroxy-8-azabicyclo[3.2.1]octane-8-carboxylate;
1-(4-amino-2-fluorophenyl)piperidin-3-yl-3-hydroxy-8-azabicyclo[3.2.1]octane-8-carboxylate;
1-2-fluoro-4-[(isopropoxycarbonyl)amino]phenylpiperidin-3-yl-3-hydroxy-8-azabicyclo[3.2.1]octane-8-carboxylate;
1-2-fluoro-4-[(methoxycarbonyl)amino]phenylpiperidin-3-yl-3-hydroxy-8-azabicyclo[3.2.1]octane-8-carboxylate;
1-4-[(ethoxycarbonyl)amino]-2-fluorophenylpiperidin-3-yl-3-hydroxy-8-azabicyclo[3.2.1]octane-8-carboxylate;
1-2-fluoro-4-[(propoxycarbonyl)amino]phenylpiperidin-3-yl-3-hydroxy-8-azabicyclo[3.2.1]octane-8-carboxylate;
1-2-fluoro-4-[(isobutoxycarbonyl)amino]phenylpiperidin-3-yl-3-hydroxy-8-azabicyclo[3.2.1]octane-8-carboxylate;
1-[2-fluoro-4-(2-oxopyrrolidin-1-yl)phenyl]piperidin-3-yl-3-hydroxy-8-azabicyclo[3.2.1]octane-8-carboxylate;
1-[2-fluoro-4-(2-oxo-1,3-oxazolidin-3-yl)phenyl]piperidin-3-yl-3-hydroxy-8-azabicyclo[3.2.1]octane-8-carboxylate;
1-(4-cyano-2-fluorophenyl)piperidin-3-yl piperidine-1-carboxylate;
1-(4-cyano-2-fluorophenyl)piperidin-3-yl 4-hydroxypiperidine-1-carboxylate;
1-(4-cyano-2-fluorophenyl)piperidin-3-yl-3-hydroxy-8-azabicyclo[3.2.1]octane-8-carboxylate;
1-4-[(cyclohexylcarbonyl)amino]-2-fluorophenylpiperidin-3-yl-3-hydroxy-8-azabicyclo[3.2.1]octane-8-carboxylate;
1-4-[(cyclopentylcarbonyl)amino]-2-fluorophenylpiperidin-3-yl-3-hydroxy-8-azabicyclo[3.2.1]octane-8-carboxylate;
1-4-[(cyclobutylcarbonyl)amino]-2-fluorophenylpiperidin-3-yl-3-hydroxy-8-azabicyclo[3.2.1]octane-8-carboxylate;
1-4-[(cyclopropylcarbonyl)amino]-2-fluorophenylpiperidin-3-yl-3-hydroxy-8-azabicyclo[3.2.1]octane-8-carboxylate;
1-[4-(cyclopentanecarbonyl-amino)-2-fluoro-phenyl]-piperidin-3-yl piperidine-1-carboxylate;
1-(4-cyano-2,6-difluorophenyl)piperidin-3-yl-piperidine-1-carboxylate;
1-(4-cyano-2,6-difluorophenyl)piperidin-3-yl-4-hydroxypiperidine-1-carboxylate;
1-(4-cyano-2,6-difluorophenyl)piperidin-3-yl-3-hydroxy-8-azabicyclo[3.2.1]octane-8-carboxylate;
1-(4-cyano-2-fluorophenyl)piperidin-3-yl-3-hydroxy-9-azabicyclo[3.3.1]nonane-9-carboxylate;
1-(2,4-difluorophenyl)piperidin-3-yl-piperidine-1-carboxylate;
1-(2,4-difluorophenyl)piperidin-3-yl-4-hydroxypiperidine-1-carboxylate;
1-(2,4-difluorophenyl)piperidin-3-yl-3-hydroxy-8-azabicyclo[3.2.1]octane-8-carboxylate;
1-(2,4-difluorophenyl)piperidin-3-yl-3-hydroxy-9-azabicyclo[3.3.1]nonane-9-carboxylate;
1-(2-fluoro-4-methylphenyl)piperidin-3-yl-piperidine-1-carboxylate;
1-(2-fluoro-4-methylphenyl)piperidin-3-yl-4-hydroxypiperidine-1-carboxylate;
1-(2-fluoro-4-methylphenyl)piperidin-3-yl-3-hydroxy-8-azabicyclo[3.2.1]octane-8-carboxylate;
1-(2-fluoro-4-methylphenyl)piperidin-3-yl-3-hydroxy-9-azabicyclo [3.3.1]nonane-9-carboxylate;
1-(3-methyl-5-nitropyridin-2-yl)piperidin-3-yl-3-hydroxy-8-azabicyclo[3.2.1]octane-8-carboxylate;
1-(5-amino-3-methylpyridin-2-yl)piperidin-3-yl-3-hydroxy-8-azabicyclo[3.2.1]octane-8-carboxylate;
1-5-[(methoxycarbonyl)amino]-3-methylpyridin-2-ylpiperidin-3-yl-3-hydroxy-8-azabicyclo[3.2.1]octane-8-carboxylate;
1-5-[(ethoxycarbonyl)amino]-3-methylpyridin-2-ylpiperidin-3-yl-3-hydroxy-8-azabicyclo[3.2.1]octane-8-carboxylate;
1-3-methyl-5-[(propoxycarbonyl)amino]pyridin-2-ylpiperidin-3-yl-3-hydroxy-8-azabicyclo[3.2.1]octane-8-carboxylate;
1-5-[(isopropoxycarbonyl)amino]-3-methylpyridin-2-ylpiperidin-3-yl-3-hydroxy-8-azabicyclo[-3.2.1]octane-8-carboxylate;
1-5-[(isobutoxycarbonyl)amino]-3-methylpyridin-2-ylpiperidin-3-yl-3-hydroxy-8-azabicyclo[3.2.1]octane-8-carboxylate;
1-(4-cyano-2-fluorophenyl)piperidin-3-yl 2-oxa-6-azatricyclo[3.3.1.1(3,7)]decane-6-carboxylate;
1-(2-fluoro-4-nitrophenyl)piperidin-3-yl 2-oxa-6-azatricyclo[3.3.1.1(3,7)]decane-6-carboxylate;
1-(2-fluoro-4-methylphenyl)piperidin-3-yl 2-oxa-6-azatricyclo[3.3.1.1(3,7)]decane-6-carboxylate;
1-(2,4-difluorophenyl)piperidin-3-yl 2-oxa-6-azatricyclo[3.3.1.1(3,7)]decane-6-carboxylate;
1-(4-amino-2-fluorophenyl)piperidin-3-yl 2-oxa-6-azatricyclo[3.3.1.1(3,7)]decane-6-carboxylate;
1-2-fluoro-4-[(methoxycarbonyl)amino]phenylpiperidin-3-yl-2-oxa-6-azatricyclo[3.3.1.1(3,7)]decane-6-carboxylate;
1-4-[(ethoxycarbonyl)amino]-2-fluorophenylpiperidin-3-yl-2-oxa-6-azatricyclo[3.3.1.1(3,7)]decane-6-carboxylate;
1-2-fluoro-4-[(propoxycarbonyl)amino]phenylpiperidin-3-yl-2-oxa-6-azatricyclo[3.3.1.1 (3,7)]decane-6-carboxylate;
1-2-fluoro-4-[(isopropoxycarbonyl)amino]phenylpiperidin-3-yl-2-oxa-6-azatricyclo[3.3.1.1 (3,7)]decane-6-carboxylate;
1-[2-fluoro-4-(isobutyrylamino)phenyl]piperidin-3-yl-2-oxa-6-azatricyclo[3.3.1.1(3,7)]decane-6-carboxylate;
1-(4-bromo-2-fluorophenyl)piperidin-3-yl-3-hydroxy-8-azabicyclo [3.2.1]octane-8-carboxylate;
1-[2-fluoro-4-(2-oxopyrrolidin-1-yl)phenyl]piperidin-3-yl 2-oxa-6-azatricyclo[3.3.1.1(3,7)]decane-6-carboxylate;
1-[2-fluoro-4-(2-oxo-1,3-oxazolidin-3-yl)phenyl]piperidin-3-yl 2-oxa-6-azatricyclo[3.3.1.1(3,7)]decane-6-carboxylate;
1-[2-fluoro-4-(2-oxo-1,3-oxazinan-3-yl)phenyl]piperidin-3-yl 2-oxa-6-azatricyclo[3.3.1.1(3,7)]decane-6-carboxylate;
1-[2-fluoro-4-(2-oxopiperidin-1-yl)phenyl]piperidin-3-yl 2-oxa-6-azatricyclo[3.3.1.1(3,7)]decane-6-carboxylate;
1-2-fluoro-4-[(isobutoxycarbonyl)amino]phenylpiperidin-3-yl-2-oxa-6-azatricyclo[3.3.1.1 (3,7)]decane-6-carboxylate;
1-(2-fluorophenyl)piperidin-3-yl-3-hydroxy-8-azabicyclo[3.2.1]octane-8-carboxylate;
1-(2-fluoro-4-6-[(methylamino)carbonyl]pyridin-3-ylphenyl)piperidin-3-yl-3-hydroxy-8-azabicyclo[3.2.1]octane-8-carboxylate;
1-(2-fluoro-4-pyridin-3-ylphenyl)piperidin-3-yl-3-hydroxy-8-azabicyclo[3.2.1]octane-8-carboxylate;
1-(2-fluoro-4-pyridin-4-ylphenyl)piperidin-3-yl-3-hydroxy-8-azabicyclo[3.2.1]octane-8-carboxylate;
1-(2-fluoro-4-pyrimidin-5-ylphenyl)piperidin-3-yl-3-hydroxy-8-azabicyclo[3.2.1]octane-8-carboxylate;
1-[2-fluoro-4-(1-methyl-1H-pyrazol-4-yl)phenyl]piperidin-3-yl-3-hydroxy-8-azabicyclo[3.2.1]octane-8-carboxylate;
1-4′-[(cyclopropylamino)carbonyl]-3-fluorobiphenyl-4-ylpiperidin-3-yl-3-hydroxy-8-azabicyclo[3.2.1]octane-8-carboxylate;
1-(4-6-[(dimethylamino)carbonyl]pyridin-3-yl-2-fluorophenyl)piperidin-3-yl-3-hydroxy-8-azabicyclo[3.2. 1]octane-8-carboxylate;
1-(4-6-[(ethylamino)carbonyl]pyridin-3-yl-2-fluorophenyl)piperidin-3-yl -3-hydroxy-8-azabicyclo[3.2. 1]octane-8-carboxylate;
1-(4-6-[(diethylamino)carbonyl]pyridin-3-yl-2-fluorophenyl)piperidin-3-yl-3-hydroxy-8-azabicyclo[3.2.1]octane-8-carboxylate;
1-[4′-(aminocarbonyl)-3-fluorobiphenyl-4-yl]piperidin-3-yl-3-hydroxy-8-azabicyclo[3.2.1]octane-8-carboxylate;
3,5-difluoro-4-(3-2-[3-hydroxy-8-azabicyclo[3.2.1]oct-8-yl]-2-oxoethylpiperidin-1-yl)benzonitrile;
8-[1-(2-fluoro-4-nitrophenyl)piperidin-3-yl]acetyl-8-azabicyclo[3.2.1]octan-3-ol;
8-[1-(4-amino-2-fluorophenyl)piperidin-3-yl]acetyl-8-azabicyclo[3.2.1]octan-3-ol;
methyl [3-fluoro-4-(3-2-[3-hydroxy-8-azabicyclo[3.2.1]oct-8-yl]-2-oxoethypiperidin-1-yl)phenyl]carbamate;
ethyl [3-fluoro-4-(3-2-[3-hydroxy-8-azabicyclo[3.2.1]oct-8-yl]-2-oxoethylpiperidin-1-yl)phenyl]carbamate;
propyl [3-fluoro-4-(3-2-[3-hydroxy-8-azabicyclo[3.2.1]oct-8-yl]-2-oxoethypiperidin-1-yl)phenyl]carbamate;
isopropyl [3-fluoro-4-(3-2-[3-hydroxy-8-azabicyclo [3.2.1]oct-8-yl]-2-oxoethypiperidin-1-yl)phenyl]carbamate;
isobutyl [3-fluoro-4-(3-2-[3-hydroxy-8-azabicyclo[3.2.1]oct-8-yl]-2-oxoethypiperidin-1-yl)phenyl]carbamate;
3-fluoro-4-(3-2-[3-hydroxy-8-azabicyclo[3.2.1]oct-8-yl]-2-oxoethylpiperidin-1-yl)benzonitrile;
8-[1-(5-chloro-3-fluoropyridin-2-yl)piperidin-3-yl]acetyl-8-azabicyclo[3.2.1]octan-3-ol;
8-(1-[4-(trifluoromethyl)pyridin-2-yl]piperidin-3-ylacetyl)-8-azabicyclo[3.2.1]octan-3ol;
8-[1-(3-chloropyridin-2-yl)piperidin-3-yl]acetyl-8-azabicyclo[3.2.1]octan-3-ol;
8-(1-[3-chloro-5-(trifluoromethyl)pyridin-2-yl]piperidin-3-ylacetyl)-8-azabicyclo[3.2.1]octan-3-ol;
1-(2-fluoro-4-methylphenyl)piperidin-3-yl-3-methylmorpholine-4-carboxylate;
1-(2,4-difluorophenyl)piperidin-3-yl-3-methylmorpholine-4-carboxylate;
1-(2,4-difluorophenyl)piperidin-3-yl-(4-hydroxycyclohexyl)methylcarbamate; and
1-(2-fluoro-4-methylphenyl)piperidin-3-yl-(4-hydroxycyclohexyl)-methylcarbamate,
or a pharmaceutically acceptable salt thereof.

40. A compound selected from:
1-(2-fluoro-4-methylphenyl)piperidin-3-yl-methyl(tetrahydro-2H-pyran-4-yl)carbamate; and
1-(2,4-difluorophenyl)piperidin-3-yl-methyl(tetrahydro-2H-pyran-4-yl)carbamate,
or a pharmaceutically acceptable salt thereof.

41. A composition comprising a compound of claim 1, or pharmaceutically acceptable salt thereof, and at least one pharmaceutically acceptable carrier.

42. A method of modulating 11βHSD1 comprising contacting said 11βHSD1 with a compound of Formula Ia or Ib: 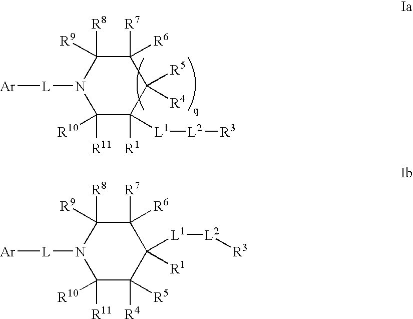
or pharmaceutically acceptable salt or prodrug thereof, wherein:
L is absent, S(O)₂, S(O), S, S(O)₂NR², C(O), C(O)O, C(O)O—(C₁₋₃ alkylene), or C(O)NR²;
L¹ is O, CH₂, or NR^{N};
L² is CO or S(O)₂;
provided that when L¹ is NR^{N}, L² is SO2;
R^{N} is H, C₁₋₆ alkyl, aryl, cycloalkyl, heteroaryl, or heterocycloalkyl;
Ar is aryl or heteroaryl, each optionally substituted by 1, 2, 3, 4 or 5 —W—X—Y-Z;
R¹ is H, C(O)OR^{b′}, S(O)R^{a′}, S(O)NR^{c′}R^{d′. S(O)}₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2 or 3 R¹⁴;
R² is H or C₁₋₆ alkyl;
R³ is H, C₁₋₆ alkyl, aryl, cycloalkyl, heteroaryl or heterocycloalkyl, wherein each of the C₁₋₆ alkyl, aryl, cycloalkyl, heteroaryl, and heterocycloalkyl is optionally substituted by 1, 2 or 3 —W′—X′—Y′-Z′;
or R³ is NR^{3a}R^{3b} or OR^{3c};
R^{3a} and R^{3b} are independently selected from H, C₁₋₆ alkyl, aryl, cycloalkyl, heteroaryl and heterocycloalkyl, wherein each of the C₁₋₆ alkyl, aryl, cycloalkyl, heteroaryl, and heterocycloalkyl is optionally substituted by 1, 2 or 3 —W′—X′—Y′-Z′;
or R^{3a} and R^{3b} together with the N atom to which they are attached form a 4-14 membered heterocycloalkyl group which is optionally substituted by 1, 2 or 3 —W′—X′—Y′-Z′;
R^{3c} is H, C₁₋₆ alkyl, aryl, cycloalkyl, heteroaryl, or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀alkynyl, aryl, cycloalkyl, heteroaryl, heterocycloalkyl, arylalkyl, heteroarylalkyl, cycloalkylalkyl, and heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2 or 3 R¹⁴;
or R¹ and R³ together with the carbon atoms to which they are attached and the intervening —NR²CO— moiety form a 4-14 membered heterocycloalkyl group which is optionally substituted by 1, 2 or 3 R¹⁴;
or R⁴ and R⁵ together with the carbon atom to which they are attached form a 3-14 membered cycloalkyl or heterocycloalkyl group which is optionally substituted by 1, 2 or 3 R¹⁴;
or R⁶ and R⁷ together with the carbon atom to which they are attached form a 3-14 membered cycloalkyl or heterocycloalkyl group which is optionally substituted by 1, 2 or 3 R¹⁴;
or R⁸ and R⁹ together with the carbon atom to which they are attached form a 3-14 membered cycloalkyl or heterocycloalkyl group which is optionally substituted by 1, 2 or 3 R¹⁴;
or R¹⁰ and R¹¹ together with the carbon atom to which they are attached form a 3-14 membered cycloalkyl or heterocycloalkyl group which is optionally substituted by 1, 2 or 3 R¹⁴;
or R⁴ and R⁶ together with the carbon atom to which they are attached form a 3-7 membered fused cycloalkyl group or 3-7 membered fused heterocycloalkyl group which is optionally substituted by 1, 2 or 3 R¹⁴;
or R⁶ and R⁸ together with the carbon atom to which they are attached form a 3-7 membered fused cycloalkyl group or 3-7 membered fused heterocycloalkyl group which is optionally substituted by 1, 2or3R¹⁴;
each R¹⁴ is independently halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of said C₁₋₆ alkylenyl, C₂₋₆ alkenylenyl, and C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl, and heterocycloalkyl, wherein each of said C₁₋₆ alkylenyl, C₂₋₆ alkenylenyl, C₂₋₆ alkynylenyl, cycloalkyl, heteroaryl, and heterocycloalkyl is optionally substituted by 1, 2 or 3 substituents independently selected from halo, CN, NO₂, OH, C₁₋₄ alkyl, C₁₋₄ haloalkyl, C₂₋₈ alkoxyalkyl, C₁₋₄ alkoxy, C₁₋₄ haloalkoxy, C₂₋₈ alkoxyalkoxy, cycloalkyl, heterocycloalkyl, C(O)OR^{a}, C(O)NR^{c}R^{d}, amino, C₁₋₄ alkylamino, and C₂₋₈ dialkylamino;
Y, Y′and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl and C₂₋₆ alkynylenyl is optionally substituted by 1, 2 or 3 independently selected from halo, OH, C₁₋₄ alkoxy, C₁₋₄ haloalkoxy, amino, C 1₄ alkylamino and C₂₋₈ dialkylamino;
Z, Z′ and Z″ are independently selected from H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl, heterocycloalkyl, halosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, C(═NR^{g})NR^{c}R^{d}, NR^{c}C(═NR^{g})NR^{c}R^{d}, S(O)R^{b}, S(O)NR^{c}R^{d}, S(O)₂R^{b}, and S(O)₂NR^{c}R^{d} wherein each of said C₁₋₆ alkyl, C₂₋₆ alkenyl, C₂₋₆ alkynyl, aryl, cycloalkyl, heteroaryl, and heterocycloalkyl is optionally substituted by 1, 2 or 3 substituents independently selected from halo, C₁₋₆ alkyl, C₂₋₆ alkenyl, C₂₋₆ alkynyl, C₁₋₄ haloalkyl, aryl, cycloalkyl, heteroaryl, heterocycloalkyl, halosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, C(═NR^{g})NR^{c}R^{d}, NR^{c}C(═NR^{g})NR^{c}R^{d}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of said C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{e} and R^{f} together with the N atom to which they are attached form a 4-, 5-, 6- or 7-membered heterocycloalkyl group;
R^{g} is H, CN, NO₂, C(O)NH₂, or C₁₋₆ alkyl; and
q is 0, 1 or 2.

43. The method of claim 42 wherein said modulating is inhibiting.

44. A method of treating a disease in a patient, wherein said disease is associated with expression or activity of 11βHSD1, comprising administering to said patient a therapeutically effective amount of Formula Ia or Ib: 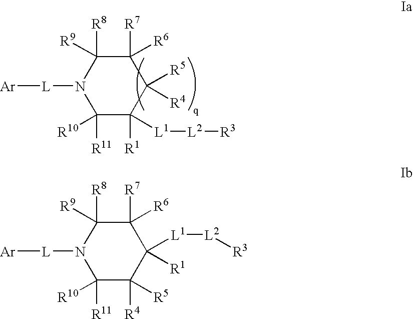
or pharmaceutically acceptable salt or prodrug thereof, wherein:
L is absent, S(O)₂, S(O), S, S(O)₂NR², C(O), C(O)O, C(O)O—(C₁₋₃ alkylene), or C(O)NR²;
L¹ is O, CH₂, or NR^{N};
L² is CO or S(O)₂;
provided that when L¹ is NR N, L² is SO2;
R^{N} is H, C₁₋₆ alkyl, aryl, cycloalkyl, heteroaryl or heterocycloalkyl;
Ar is aryl or heteroaryl, each optionally substituted by 1, 2, 3, 4 or 5 —W—X—Y-Z;
R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹⁴;
R² is H or C₁₋₆ alkyl;
R³ is H, C₁₋₆ alkyl, aryl, cycloalkyl, heteroaryl or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
or R³ is NR^{3a}R^{3b} or OR^{3c};
R^{3a} and R^{3b} are independently selected from H, C₁₋₆ alkyl, aryl, cycloalkyl, heteroaryl and heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
or R^{3a} and R^{3b} together with the N atom to which they are attached form a 4-14 membered heterocycloalkyl group which is optionally substituted by 1, 2 or 3 —W′—X′—Y′-Z′;
R^{3c} is H, C₁₋₆ alkyl, aryl, cycloalkyl, heteroaryl or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹⁴;
or R¹ and R³ together with the carbon atoms to which they are attached and the intervening —NR²CO— moiety form a 4-14 membered heterocycloalkyl group which is optionally substituted by 1, 2 or 3 R¹⁴;
or R⁴ and R⁵ together with the carbon atom to which they are attached form a 3-14 membered cycloalkyl or heterocycloalkyl group which is optionally substituted by 1, 2 or 3 R¹⁴;
or R⁶ and R⁷ together with the carbon atom to which they are attached form a 3-14 membered cycloalkyl or heterocycloalkyl group which is optionally substituted by 1, 2 or 3 R¹⁴;
or R⁸ and R⁹ together with the carbon atom to which they are attached form a 3-14 membered cycloalkyl or heterocycloalkyl group which is optionally substituted by 1, 2 or 3 R¹⁴;
or R¹⁰ and R¹¹ together with the carbon atom to which they are attached form a 3-14 membered cycloalkyl or heterocycloalkyl group which is optionally substituted by 1, 2 or 3 R¹⁴;
or R⁴ and R⁶ together with the carbon atom to which they are attached form a 3-7 membered fused cycloalkyl group or 3-7 membered fused heterocycloalkyl group which is optionally substituted by 1, 2 or 3 R¹⁴;
or R⁶ and R⁸ together with the carbon atom to which they are attached form a 3-7 membered fused cycloalkyl group or 3-7 membered fused heterocycloalkyl group which is optionally substituted by 1,2or3R¹⁴;
each R¹⁴ is independently halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of said C₁₋₆ alkylenyl, C₂₋₆ alkenylenyl and C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of said C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₄ alkyl, C₁₋₄ haloalkyl, C₂₋₈ alkoxyalkyl, C₁₋₄ alkoxy, C₁₋₄ haloalkoxy, C₂₋₈ alkoxyalkoxy, cycloalkyl, heterocycloalkyl, C(O)OR^{a}, C(O)NR^{c}R^{d}, amino, C₁₋₄ alkylamino and C₂₋₈ dialkylamino;
Y, Y′and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl and C₂₋₆ alkynylenyl is optionally substituted by 1, 2 or 3 independently selected from halo, OH, C₁₋₄ alkoxy, C₁₋₄ haloalkoxy, amino, C_{1 4} alkylamino and C₂₋₈ dialkylamino;
Z, Z′ and Z″ are independently selected from H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl, heterocycloalkyl, halosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, C(═NR^{g})NR^{c}R^{d}, NR^{c}C(═NR^{g})NR^{c}R^{d}, S(O)R^{b}, S(O)NR^{c}R^{d}, S(O)₂R^{b}, and S(O)₂NR^{c}R^{d} wherein each of said C₁₋₆ alkyl, C₂₋₆ alkenyl, C₂₋₆ alkynyl, aryl, cycloalkyl, heteroaryl, and heterocycloalkyl is optionally substituted by 1, 2 or 3 substituents independently selected from halo, C₁₋₆ alkyl, C₂₋₆ alkenyl, C₂₋₆ alkynyl, haloalkyl, aryl, cycloalkyl, heteroaryl, heterocycloalkyl, halosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)(R^{a}, C(═NR^{g})NR^{c}R^{d}, NR^{c}C(═NR^{g})NR^{c}R^{d}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″; wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of said C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, Can₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{e} and R^{f} together with the N atom to which they are attached form a 4-, 5-, 6- or 7-membered heterocycloalkyl group;
R^{g} is H, CN, NO₂, C(O)NH₂, or C₁₋₆ alkyl; and
q is 0, 1 or 2.

45. The method of claim 44 wherein said disease is obesity, diabetes, glucose intolerance, insulin resistance, hyperglycemia, hypertension, hyperlipidemia, cognitive impairment, dementia, depression, glaucoma, cardiovascular disorders, osteoporosis, inflammation, metabolic syndrome, atherosclerosis, coronary heart disease, type 2 diabetes, hypercortisolemia, androgen excess, and polycystic ovary syndrome (PCOS).

46. A method of treating obesity, diabetes, glucose intolerance, insulin resistance, hyperglycemia, hypertension, hyperlipidemia, cognitive impairment, dementia, depression, glaucoma, cardiovascular disorders, osteoporosis, inflammation, metabolic syndrome, atherosclerosis, coronary heart disease, type 2 diabetes, hypercortisolemia, androgen excess, or polycystic ovary syndrome (PCOS), comprising administering to a patient a pharmaceutically effective amount of a compound of Formula Ia or Ib: 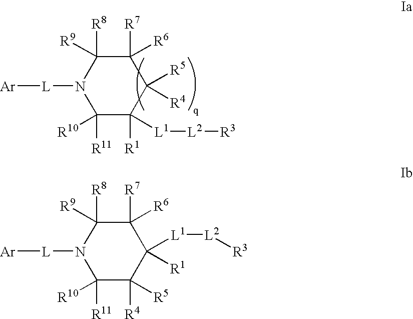
or pharmaceutically acceptable salt or prodrug thereof, wherein:
L is absent, S(O)₂, S(O), S, S(O)₂NR², C(O), C(O)O, C(O)O—(C₁₋₃ alkylene), or C(O)NR²;
L¹ is O, CH₂, or NR^{N};
L² is CO or S(O)₂;
provided that when L¹ is NR N, L2 is SO₂;
R^{N} is H, C₁₋₆ alkyl, aryl, cycloalkyl, heteroaryl or heterocycloalkyl;
Ar is aryl or heteroaryl, each optionally substituted by 1, 2, 3, 4 or 5 —W—X—Y-Z;
R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹⁴;
R² is H or C₁₋₆ alkyl;
R³ is H, C₁₋₆ alkyl, aryl, cycloalkyl, heteroaryl or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
or R³ is NR^{3a}R^{3b} or OR^{3c};
R^{3a} and R^{3b} are independently selected from H, C₁₋₆ alkyl, aryl, cycloalkyl, heteroaryl and heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
or R^{3a} and R^{3b} together with the N atom to which they are attached form a 4-14 membered heterocycloalkyl group which is optionally substituted by 1, 2 or 3 —W′—X′—Y′-Z′;
R^{3c} is H, C₁₋₆ alkyl, aryl, cycloalkyl, heteroaryl or heterocycloalkyl, wherein each of the C₁₋₆ alkyl, aryl, cycloalkyl, heteroaryl and heterocycloalkyl is optionally substituted by 1, 2 or 3 —W′—X′—Y′-Z′;
R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl, wherein each of said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl is optionally substituted by 1, 2 or 3 R¹⁴;
or R¹ and R³ together with the carbon atoms to which they are attached and the intervening —NR²CO— moiety form a 4-14 membered heterocycloalkyl group which is optionally substituted by 1, 2or3R¹⁴;
or R⁴ and R⁵ together with the carbon atom to which they are attached form a 3-14 membered cycloalkyl or heterocycloalkyl group which is optionally substituted by 1, 2 or 3 R¹⁴;
or R⁶ and R⁷ together with the carbon atom to which they are attached form a 3-14 membered cycloalkyl or heterocycloalkyl group which is optionally substituted by 1, 2 or 3 R¹⁴;
or R⁸ and R⁹ together with the carbon atom to which they are attached form a 3-14 membered cycloalkyl or heterocycloalkyl group which is optionally substituted by 1, 2 or 3 R¹⁴;
or R¹⁰ and R¹¹ together with the carbon atom to which they are attached form a 3-14 membered cycloalkyl or heterocycloalkyl group which is optionally substituted by 1, 2 or 3 R¹⁴;
or R⁴ and R⁶ together with the carbon atom to which they are attached form a 3-7 membered fused cycloalkyl group or 3-7 membered fused heterocycloalkyl group which is optionally substituted by 1,2 or 3 R¹⁴;
or R⁶ and R⁸ together with the carbon atom to which they are attached form a 3-7 membered fused cycloalkyl group or 3-7 membered fused heterocycloalkyl group which is optionally substituted by 1,2 or 3 R¹⁴;
each R¹⁴ is independently halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}S(O)₂R^{b′}, or S(O)₂NR^{c′}R^{d′};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of said C₁₋₆ alkylenyl, C₂₋₆ alkenylenyl, and C₂₋₆ alkynylenyl is optionally substituted by 1, 2 or 3 independently selected from halo, OH, C₁₋₄ alkoxy, C₁₋₄ haloalkoxy, amino, C₁₋₄ alkylamino, and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of said C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₄ alkyl, C₁₋₄ haloalkyl, C₂₋₈ alkoxyalkyl, C₁₋₄ alkoxy, C₁₋₄ haloalkoxy, C₂₋₈ alkoxyalkoxy, cycloalkyl, heterocycloalkyl, C(O)OR^{a}, C(O)NR^{c}R^{d}, amino, C₁₋₄ alkylamino, and C₂₋₈ dialkylamino;
Y, Y′and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl and C₂₋₆ alkynylenyl is optionally substituted by 1, 2 or 3 independently selected from halo, OH, C₁₋₄ alkoxy, C₁₋₄ haloalkoxy, amino, C₁₋₄ alkylamino, and C₂₋₈ dialkylamino;
Z, Z′ and Z″ are independently selected from H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl, heterocycloalkyl, halosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, C(═NR^{g})NR^{c}R^{d}, NR^{c}C(═NR^{g})NR^{c}R^{d}, S(O)R^{b}, S(O)NR^{c}R^{d}, S(O)₂R^{b}, and S(O)₂NR^{c}R^{d} wherein each of said C₁₋₆ alkyl, C₂₋₆ alkenyl, C₂₋₆ alkynyl, aryl, cycloalkyl, heteroaryl, and heterocycloalkyl is optionally substituted by 1, 2 or 3 substituents independently selected from halo, C₁₋₆ alkyl, C₂₋₆ alkenyl, C₂₋₆ alkynyl, haloalkyl, aryl, cycloalkyl, heteroaryl, heterocycloalkyl, halosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, C(NR^{g})NR^{c}R^{d}, NR^{c}C(═NR^{g})NR^{c}R^{d}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of said C₁₋₆ alkyl, C₁₋₆ haloalkyl, C₂₋₆ alkenyl, C₂₋₆ alkynyl, aryl, cycloalkyl, heteroaryl, and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, heteroaryl, cycloalkyl, heterocycloalkyl, arylalkyl, heteroarylalkyl, cycloalkylalkyl, and heterocycloalkylalkyl is optionally substituted by OH, amino, halo, C₁₋₁₆ alkyl, C₁₋₁₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{e} and R^{f} together with the N atom to which they are attached form a 4-, 5-, 6- or 7-membered heterocycloalkyl group;
R^{g} is H, CN, NO₂, C(O)NH₂, or C₁₋₆ alkyl; and
q is 0, 1 or 2.
